(19) United States
(12) Patent Application Publication
(10) Pub. No.: US 2007/0088414 A1
(43) Pub. Date: Apr. 19, 2007

(54) Particulate formulations for intradermal delivery of biologically active agents

(76) Inventors: Robert L. Campbell, Bahama, NC (US); Kevin G. Dolan, Holly Springs, NC (US); Wendy D. Woodley, Cary, NC (US)

Correspondence Address: BECTON, DICKINSON AND COMPANY;ALSTON & BIRD LLP, 1 BECTON DRIVE, MC 110, FRANKLIN LAKES, NJ, 07417-1880, US

(21) Appl. No.: 11/442,516

(22) Filed: May 25, 2006

Related U.S. Application Data
(60) Provisional application No. 60/684,161, filed on May 25, 2005.
(60) Provisional application No. 60/782,754, filed on Mar. 15, 2006.

(51) Int. Cl. A61F 2/00

(52) U.S. Cl. 607/101

(57) ABSTRACT

The present invention relates to formulations, methods and devices for delivering one or more biologically active agents, particularly a diagnostic or therapeutic agent to the intradermal compartment of a subject's skin. The present invention provides an improved method of delivery of biologically active agents in that it provides among other benefits, rapid uptake into the local lymphatics, improved targeting to a particular tissue, improved bioavailability, improved tissue bioavailability, improved tissue specific kinetics, improved deposition of a pre-selected volume of the agent to be administered. This invention provides methods for rapid transport of agents through lymphatic vasculature accessed by intradermal delivery of the agent. Methods of the invention are particularly useful for delivery of diagnostic and therapeutic agents. The invention relates to the synergy gained in diagnosing and treating disease when intradermal delivery and controlled release materials are combined. Specifically, the synergy is achieved when intradermal delivery is combined with lipid based particles.

This application claims the benefit of U.S. Provisional Application No. 60/684,161, filed May 25, 2005, and U.S. Provisional Application No. 60/782,754, filed Mar. 15, 2006, both of which are incorporated herein by reference in their entireties.

### 1. FIELD OF THE INVENTION

The present invention relates to formulations, methods and devices for delivering one or more biologically active agents, particularly diagnostic and/or therapeutic agent(s) to the intradermal compartment of a subject's skin. The present invention provides an improved method of delivery of biologically active agent(s) in that it provides among other benefits, rapid uptake into the local lymphatics, improved targeting to a particular tissue, improved bioavailability, improved tissue bioavailability, improved tissue specific kinetics, improved deposition of a pre-selected volume of the agent to be administered, and rapid biological pharmacodynamics and biological pharmacokinetics. This invention provides methods for rapid transport of agents through lymphatic vasculature accessed by intradermal delivery of the agent. Methods of the invention are particularly useful for delivery of diagnostic and therapeutic agents. The methods and formulations described within prevent the accumulation of toxic agents in critical organs.

### 2. BACKGROUND OF THE INVENTION

2.1 Delivery of Agents to the Skin

The importance of efficiently and safely administering pharmaceutical agents such as diagnostic agents and drugs has long been recognized. Difficulties associated with ensuring adequate bioavailability and reproducible absorption of large molecules, such as proteins that have arisen out of the biotechnology industry, have been recently highlighted (Cleland et al., Curr. Opin. Biotechnol. 12: 212-219, 2001). The use of conventional needles has long provided one approach for delivering pharmaceutical agents to humans and animals by administration through the skin. In general, injection avoids harsh conditions associated with oral delivery that commonly mitigate the desired effects of most biological therapies. Injection may also provide faster therapeutic effect than oral administration. Considerable effort has been made to achieve reproducible and efficacious delivery needle-based injection while improving the ease of use and reducing patient apprehension and/or pain associated with conventional needles. Furthermore, certain transcutaneous delivery systems eliminate needles entirely, and rely upon simple hydrophobic adsorption, chemical mediators or external driving forces such as iontophoretic currents or electroporation or thermal poration or sonophoresis to breach the stratum corneum (the outermost layer of the skin) and deliver agents through the surface of the skin. However, such delivery systems do not, in general, reproducibly traverse the skin barriers or deliver pharmaceutical agents to a given depth below the surface of the skin. Consequently, clinical results can be variable. Thus, mechanical breach of the stratum corneum, such as with needles, is believed to provide the most reproducible method of administration of agents through the surface of the skin, and provides control and reliability in the placement of the administered agents.

Approaches for delivering agents beneath the surface of the skin have almost exclusively involved transdermal injections or infusions, i.e., delivery of agents through the skin to a site beneath the skin. Transdermal injections and infusions include subcutaneous, intramuscular or intravenous routes of administration of which, intramuscular (IM) and subcutaneous (SC) injections have been the most commonly used.

Anatomically, the outer surface of the body is made up of two major tissue layers, an outer epidermis and an underlying dermis, which together constitute the skin (for review, see Physiology, Biochemistry, and Molecular Biology of the Skin, Second Edition, L. A. Goldsmith, Ed., Oxford University Press, New York, 1991). The epidermis is subdivided into five layers or strata of a total thickness of between 75 and 150 μm. Beneath the epidermis lies the dermis, which contains two layers, an outermost portion referred to as the papillary dermis and a deeper layer referred to as the reticular dermis. The papillary dermis contains vast microcirculatory blood and lymphatic plexuses. In contrast, the reticular dermis is relatively acellular and avascular and made up of dense collagenous and elastic connective tissue. Beneath the epidermis and dermis is the subcutaneous tissue, also referred to as the hypodermis, which is composed of connective tissue and fatty tissue. Muscle tissue lies beneath the subcutaneous tissue.

As noted above, both the subcutaneous tissue and muscle tissue have been commonly used as sites for administration of pharmaceutical agents, including diagnostic agents. The dermis, however, has rarely been targeted as a site for administration of agents, and this may be due, at least in part, to the difficulty of precise needle placement into the intradermal compartment. Furthermore, even though the dermis, in particular, the papillary dermis has been known to have a high degree of vascularity, it has not heretofore been appreciated that one could take advantage of this high degree of vascularity to obtain an improved absorption profile for administered agents compared to subcutaneous administration.

One approach to administration beneath the surface to the skin and into the region of the intradermal compartment has been routinely used in the Mantoux tuberculin test. In this procedure, a purified protein derivative is injected at a shallow angle to the skin surface using a 27 or 30 gauge needle (Flynn et al., Chest 106: 1463-5, 1994). A degree of uncertainty in placement of the injection can, however, result in some false negative test results. Moreover, the test has involved a localized injection to elicit a response at the site of injection and the Mantoux approach has not led to the use of intradermal injection for systemic administration of agents.

Some groups have reported on systemic administration by what has been characterized as “intradermal” injection. In one such report, a comparison study of subcutaneous and what was described as “intradermal” injection was performed (Autret et al., Therapie 46:5-8, 1991). The pharmaceutical agent tested was calcitonin, a protein of a molecular weight of about 3600. Although it was stated that the drug was injected intradermally, the injections used a 4 mm needle pushed up to the base at an angle of 60°. This would have resulted in placement of the injectate at a depth of about 3.5 mm and into the lower portion of the reticular dermis or into the subcutaneous tissue rather than into the vascularized papillary dermis. If, in fact, this group injected into the lower portion of the reticular dermis rather than into the subcutaneous tissue, it would be expected that the agent would either be slowly absorbed in the relatively less vascular reticular dermis or diffuse into the subcutaneous region to result in what would be functionally the same as subcutaneous administration and absorption. Such actual or functional subcutaneous administration would explain the reported lack of difference between subcutaneous and what was characterized as intradermal administration, in the times at which maximum plasma concentration was reached, the concentrations at each assay time and the areas under the curves.

Similarly, Bressolle et al., administered sodium ceftazidime in what was characterized as “intradermal” injection using a 4 mm needle (Bressolle et al., J. Pharm. Sci. 82:1175-1178, 1993). This would have resulted in injection to a depth of 4 mm below the skin surface to produce actual or functional subcutaneous injection, although good subcutaneous absorption would have been anticipated in this instance because sodium ceftazidime is hydrophilic and of relatively low molecular weight.

Another group reported on what was described as intradermal drug delivery device (U.S. Pat. No. 5,007,501). Injection was indicated to be at a slow rate and the injection site was intended to be in some region below the epidermis, i.e., the interface between the epidermis and the dermis or the interior of the dermis or subcutaneous tissue. This reference, however, provided no teachings that would suggest a selective administration into the dermis nor did the reference suggest any possible pharmacokinetic advantage that might result from such selective administration.

It has also been reported that when therapeutic substances are delivered to the intradermal compartment such that both the rate of delivery and the pressure is controlled, using a needle with a length and an outlet depth within the intradermal compartment, an improved pharmacokinetic profile may be obtained as compared to delivery of the same substance to the subcutaneous compartment (disclosed in U.S. patent application Ser. No. 09/606,909, filed on Jun. 29, 2000; U.S. Patent Publication Nos. 2002-0156453 A1, published Oct., 24, 2002; 2002-0095134 A1, published Jul. 18, 2002; 2003-0100885 A1, published May 29, 2003; all of which are incorporated herein by reference in their entirety). Despite the discovery that improved pharmacokinetic profiles may be obtained, there remains a continuing need for efficient and safe formulations, methods and devices for administration of pharmaceutical agents, especially diagnostic agents.

2.2 Delivery of Agents for Diagnosis or Treatment of Diseases

Cancer is one of the most significant chronic conditions. The American Cancer Society's Cancer Facts and Figures, 2003 indicates over 1.3 million Americans will receive a cancer diagnosis this year. In the US, cancer is second only to heart disease in mortality accounting for one of four deaths. In 2002, the National Institutes of Health estimated total costs of cancer totaled $171.6 billion, with $61 billion in direct expenditures. Incidence of cancer is widely expected to increase as the US population ages, further augmenting the impact of this condition. The current treatment regimens of chemotherapy and radiation essentially established in the 1970s and 1980s, have not changed dramatically. These treatments have limited utility since they are relatively nonspecific, affecting processes in both normal and cancer cells. Another reason for the continued slow progress in treating cancer is that it arises primarily as a result of a breakdown in regulation at the molecular and cellular level. Although scientific understanding of cell regulatory processes is accelerating, the benefits of this knowledge are critically dependent on early detection and profiling of cancer at the cellular and molecular level in the clinic.

Many efforts have been focused on improving the detection of cancer. One recent advance in identifying cancer and its spread is the Sentinel Lymph Node Biopsy and Mapping procedure. Generally, this surgical procedure identifies the lymphatic network that drains the area in and around a tumor. Mapping this network allows the surgeon to visualize the patient's lymphatic system, aiding in the detection of cancerous growths and determining the lymphatic involvement in the disease. Diseased tissue and involved lymph nodes can be removed with greater efficiency and accuracy. The placement and number of involved lymph nodes affect subsequent treatment decisions. This is especially important for breast cancer patients. The sentinel mapping procedure employs intradermal delivery of a radioisotope-labeled tracer and a dye. The dye provides the visual enhancement while the tracer assists in identifying the sentinel lymph nodes that first drain from the tumor tissue. The tissue and nodes, once removed, are quickly evaluated by a waiting pathologist who examines the nodes and makes gross evaluations concerning cancer involvement. For the most part, macrometastasis can be identified, while micrometastasis requires a more lengthy examination post surgery. Together, the surgeon and pathologist decide how much additional tissue, as well as how many of the lymph nodes, are to be removed.

One problem with the current Sentinel Node Biopsy and Mapping procedure is its lack of sensitivity and specificity. Identification of cancer invasion into the lymph node is done by gross observation. Micrometastasis cannot be detected during the procedure. The reagents used are non-specific and do not aid in identifying rare cells. Addition of specific reagents in this manner improves sensitivity by giving the histologist and surgeon a more specific and sensitive signal that will allow for identification of rare cells in the tissue. Intradermal delivery of these reagents has been developed and used to substitute subcutaneous delivery, because intradermal delivery eliminates background signal from the tissue surrounding the lymph nodes (disclosed in U.S. Pat. Pub. No. 2005-0163711, published Jul. 28, 2005; and incorporated herein by reference in its entirety). The current manual intradermal delivery works for reducing the background signal due to dye in non-lymphatic tissues. Despite obvious advantages, the skill and experience required to reliably perform sentinel node biopsies is a significant barrier to widespread clinical use. Infectious diseases similarly account for significant morbidity and mortality. For example, the CDC estimates 42 million people are infected with HIV worldwide. Present diagnostic methods generally rely on in vitro assay for diagnostic profiling. However, information regarding disease loci is therefore lost. This information is potentially important for staging and therapy selection.

The present invention describes novel formulations methods and devices for profiling and treating disease, including infections using encapsulated and controlled release agents, with and without targeting ligands.

### 3. SUMMARY OF THE INVENTION

The present invention provides a method for administering one or more biologically active agents, including diagnostic and therapeutic agents, to a subject's skin, in which the agent is delivered to the intradermal (ID) compartment of the subject's skin. The present invention is based in part, on the unexpected discovery by the inventors that when such agents are delivered to the ID compartment they are transported to the local lymphatic system rapidly and efficiently as compared to conventional modes of delivery, including subcutaneous delivery and intravenous delivery, and thus provide the benefits disclosed herein. Although not intending to be bound by a particular mechanism of action, agents delivered in accordance with the methods of the invention are transported in vivo through the local lymphatic system. Although not intending to be bound by a particular mechanism of action, agents delivered in accordance with the methods of the invention introduce to the subject a condition that causes particles containing the agent to aggregate subsequent to the delivery of said particles to the intradermal compartment, wherein said aggregates are of sufficient size to be retained by lymphatic tissue, minimizing accumulation in central organs.

In one embodiment, this invention encompasses formulations comprising particulates (e.g., encapsulated or controlled-release) containing active agent for intradermal delivery to improve the therapeutic or diagnostic characteristics of the active agent, and methods of intradermally delivering the formulations to a subject. Examples of particulates, include liposomes, nanoshells, fullerines, dendrimers, quantum dots and microspheres formed with PGLA or silicon. The particle may have an outer wall, a shell, an outer layer or an outer bilayer to facilitate encapsulation of smaller molecules. Encapsulated or controlled-release formulations of the active agent may form a sac, including, but not limited to, a lipid-based sac, e.g., liposome. The liposome may incorporate polymers and/or be polymerized (polymersomes). The liposomes may have ligands or binding type molecules attached to the surface that can be used for targeting the diagnostic or therapeutic particle. The binder can also be leveraged to transform the particle in vivo.

Upon intradermal delivery according to methods of this invention, and upon introducing a condition that causes the particles containing an active agent to aggregate, the particles form aggregates the sizes of which are sufficiently large to be retained by lymphatic tissue. The particles may be a sac (e.g., liposome) or a microcapsule.

The aggregates may be formed by the introduction of a variety of conditions, including but not limited to, a condensing agent, heat, sonowave, or magnetic force. The aggregates may also be formed using surface-modified particulates, e.g., coating the particulate, e.g., liposome, surface with a binder molecule which has affinity for another molecule. In some embodiments, liposome molecules with specific size, charge, and/or loading capacity may be retained by lymphatic tissue without any external conditions applied after the administration.

Liposome particles of a broad range of size, charge, or payload (active agent to Pi ratio, as determined using the methods described herein) may be used in connection with the methods of the invention. In one embodiment, liposome particles having a size of from about 10 nm to about 17,000 nm, from about 10 nm to about 6450 nm, from about 10 nm to about 2310 nm, from about 10 nm to about 770 nm, or from about 10 nm to about 300 nm, as determined using the methods described herein, are used in connection with the formulations and methods of the invention.

In another embodiment, liposome particles having a charge of from about −1 mV zeta potential to about −80 mV zetapotential, from about −10 mV to about −80 mV, from about −10 mV to about −70 mV, from about −15 mV to about −60 mV, from about −20 mV to about −60 mV, from about −10 mV to about −40 mV, from about −30 mV to about −50 mV, from about −20 mV to about −40 mV, or form about −60 mV to about −80 mV, as determined using the methods described herein, are used in connection with the formulations and methods of the invention. In particular embodiments, liposome particles having a charge of about −1 mV to about −20 mV or about −60 mV to about −17 mV may be used. Zeta potential analyses are known to those skilled in the art of nanoparticle manufacturing. Zeta potential analyses may be attempted with the existing particle storage buffer or may be diluted in water where only trace amounts of the original storage buffer components are present during analyses. The starting buffers for the particles described herein are preferably: 2% V/V glycerol—20 mM EDTA—20 mM Tris Base—0.2% Na Azide—adjusted to pH 7.4 —adjusted to 310 mOSM; and/or 10 mM PIPES—20 g/L mannitol—0.2% Na Azide—pH adjusted to 6.8.

In one embodiment, the encapsulated active agent used in connection with the formulations and methods of the invention may have a molecular weight of from about 300, preferably 575 to 600, more preferably 586, to about 150,000 to 160,000, more preferably 158,000. In another embodiment, liposome particles having a payload of from about 0.01 nmoles to about 15 nmoles per 50 nmoles phosphate, preferably from about 0.02 nmoles to about 11 nmoles per 50 nmoles phosphate, more preferably from about 0.024 nmoles to about 10.956 nmoles per 50 nmoles phosphate, as determined using the methods described herein, are used in connection with the formulations and methods of the invention. In regards to diagnostics, the particle is sometimes referred to as a particulate label and containing a detector molecule. The diagnostic or detector agent may comprise 0.8% of the particle on a molar basis, preferably 4% and most preferred is 20%. A treatment or therapeutic agent may represent 0.07% of the particle on a per weight basis, potentially 0.7% and often 7% is the preferred when the drug is a low molecular weight, such as a chemotoxic substance used in the treatment of cancer.

The particles of the invention may comprise varying portions of active agent, e.g., diagnostic or therapeutic agent. For example, in the case of a therapeutic agent, the particles may comprise 0.8 to 20% of the agent on a molar basis. In the case of a diagnostic agent, the particles may comprise 0.8 to 20% of the agent on a molar basis. The diagnostic or therapeutic agent is preferably entrapped or encapsulated in the particle.

A wide variety of active agents may be used in connection with the formulations and methods of the invention. Examples include, but are not limited to, a radionucleotide, a gas, a dye, an antibody, a cytokine, and a chemotoxic agent, as well as those others described herein elsewhere. Other examples include, but are not limited to, agents used in ultrasound applications such as, but not limited to, octafluoropropane, nitrogen, perfluorohexane, perfluorocarbon, sulfur hexafluoride, air, or a mixture thereof.

In some embodiments, the formulations of the invention, once delivered to a subject, can be tracked remotely, e.g. with naked eyes or with the use of an instrument. In some embodiments, the formulations of the invention may be tracked remotely using methods known in the art (e.g., use of instrumentation), as well as those described herein. The particles in the encapsulated or controlled-release formulations of the invention, once in vivo, undergo a transformation to allow a better retention in the lymphatic or other targeted tissue, such as a tumor. In one embodiment, the transformation is change in shape. In another embodiment, the transformation is a change in outer diameter (e.g., size) obtained through, for example, aggregation of the particles.

The present invention provides an improved method of delivery of biologically active agents, in that it may provide among other benefits, rapid uptake into the local lymphatics, improved targeting to a particular tissue, i.e., improved deposition of the delivered agent into the particular tissue, i.e., group or layer of cells that together perform a specific function, improved systemic bioavailability, improved tissue bioavailability, improved deposition of a pre-selected volume of the agent to be administered, improved tissue-specific kinetics. Such benefits of the invention are improved over other methods of delivering biologically active agents which deposit the agent into deeper tissue compartments than the intradermal compartment including, for example, subcutaneous compartment and intravenous injection. Such benefits of the methods of the invention may be especially useful for the delivery of diagnostic and therapeutic agents. Without being limited by a particular theory, intradermal delivery of a diagnostic agent in accordance with the formulations, devices and methods of the invention deposits the diagnostic agent into the intradermal and lymphatic compartments, thus creating a rapid and biologically significant concentration of the diagnostic agent in these compartments. Rapid diagnostics can therefore be performed using less diagnostic agent. The methods of the invention are also advantageous for the delivery of therapeutic agents, where the advantages preferably include the reduction of toxic therapeutic agents and toxic side affects.

Intradermally delivered biologically active agents preferably have improved tissue bioavailability in a particular tissue, including but not limited to, skin tissue, lymphatic tissue (e.g., lymph nodes). The delivery of a biologically active agent in accordance with the methods of the invention is believed to result in improved tissue bioavailability as compared to when the same agent is delivered to the subcutaneous (SC) compartment or when the same agent is delivered by the intravenous method. Improved tissue bioavailability of agents delivered in accordance with the methods of the invention is particularly useful when delivering diagnostic or therapeutic agents to the ID compartment, as it may reduce the amount of the diagnostic and therapeutic agent required for each diagnostic or treatment procedure, which may be difficult and costly to obtain. The reduced amount of the diagnostic and therapeutic agent further reduces the likelihood of side effects associated with the procedure, e.g., toxicity.

The improved tissue bioavailability of the agents delivered in accordance with the methods of the invention can be determined using methods and parameters known to those skilled in the art, for example, by measuring the total amount of the agent accumulated in a particular tissue using, for example, histological methods known to those skilled in the art and disclosed herein. Alternatively, improved tissue bioavailability of the agents can be assessed as the amount of the agent presented to the particular tissue, the amount of the agent accumulated per mass or volume of a particular tissue, amount of the agent accumulated per unit time in a particular mass or volume of a particular tissue. For example, in one embodiment, the amount of a drug, e.g., platinum, achieved in lymph node may range from about 0.1 to 12 μg per gram of lymph node.

Encapsulated and/or controlled release versions of biologically active agents delivered in accordance with the methods of the invention are deposited in the intradermal compartment and first distributed with high bioavailability to the lymphatic tissue local to the administration site, followed by a more wide spread lymphatic delivery. In some embodiments, the methods of the present invention are particularly effective for diagnosis and treatment of a disease or disorder.

Intradermally delivered encapsulated and/or controlled release formulations of biologically active agents, especially diagnostic and therapeutic agents, may exhibit more rapid onset versus conventional delivery including SC delivery. The methods of the invention, therefore, may confer several advantages when preferably delivering encapsulated and/or controlled release agents to the ID compartment of a subject's skin. First, the methods disclosed herein may reduce potential side effects and discomfort due to the diagnostic or therapeutic procedures. Second, they may enable the rapid and repeated trial of sequential procedures in a single diagnostic or treatment session. Third, they may reduce the time required in expensive medical or imaging facilities. Fourth, they may facilitate real time studies of physiology. Fifth, they may reduce potential background signal generated by unbound and un-cleared diagnostic reagents. Sixth, patients may experience reduced pain from the methods of the invention in comparison to pain perceived from IV administration, SC injection, Mantoux injection, or surgical biopsy.

Delivering encapsulated and/or controlled release formulations of biologically active agents, including diagnostic and therapeutic agents, in accordance with the methods of the invention is preferred over traditional modes of delivery including SC delivery and intravenous delivery because the amount or percentage of the pre-selected dose of the agent that accumulates in the lymphatic tissue is increased, as measured, for example, using histopathological methods or other methods known to one skilled in the art, such as ELISA, RIA, HPLC, ICP-MS and imaging methods disclosed herein.

As used herein, delivery to the intradermal compartment or intradermally delivered is intended to mean administration of a biologically active agent into the dermis in such a manner that the agent readily reaches the richly vascularized papillary dermis and is rapidly absorbed into lymphatic vessels. Such can result from placement of the agent in the upper region of the dermis, i.e., the papillary dermis or in the upper portion of the relatively less vascular reticular dermis such that the agent readily diffuses into the papillary dermis. The controlled delivery of a biologically active agent in this dermal compartment below the papillary dermis in the reticular dermis, but sufficiently above the interface between the dermis and the subcutaneous tissue, should enable an efficient (outward) migration of the agent to the (undisturbed) vascular and lymphatic microcapillary bed (in the papillary dermis). In some embodiments, placement of a biologically active agent predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption of the agent. Although not intending to be bound by a particular mechanism of action, placement of the biologically active agent predominately at greater depths and/or into the lower portion of the reticular dermis may result in less effective uptake of the agent by the lymphatics, as the agent is slowly absorbed in the less vascular reticular dermis or in the subcutaneous compartment. For lipid-based controlled release agents, a more preferred delivery depth is from about 0.3 mm to about 1.25 mm, preferably about 1 mm.

In some embodiments, encapsulated and/or controlled release biologically active agents, including diagnostic and therapeutic agents, delivered in accordance with the methods of the invention achieve higher maximum tissue concentrations (tissue Cₘₐₓ) of the agents and allow reduced overall dosing. Therefore, the dose may be reduced, providing an economic benefit, as well as a physiological benefit since lesser amounts of the diagnostic or therapeutic agent has to be cleared by the body.

The improved benefits associated with ID delivery of encapsulated and/or controlled release biologically active agents in accordance with the methods of the invention can be achieved using not only microdevice-based injection systems. In specific embodiments, the administration of the biologically active agent is accomplished through insertion of a needle or cannula into the intradermal compartment of the subject's skin.

The intradermal delivery of diagnostic and therapeutic agents in accordance with the present invention may be particularly beneficial in the diagnosis of diseases, including chronic and acute diseases, which include, but are not limited to, lymphoma, breast cancer, melanoma, colorectal cancer, head and neck cancer, lung cancer, cancer metastasis, including micrometastasis, viral infections, e.g., HIV, diseases or disorders of the lymphatic system, and any disease affecting the lymph nodes. Although not intending to be bound by a particular mechanism of action, diagnostic agents delivered in accordance with the methods of the invention are deposited in the intradermal compartment and taken up by the lymphatic system, where its recognition and binding of a particular cell in a particular tissue indicate the presence of a cell or disease state. The present invention is useful for diagnostic procedures including, but not limited to, surgical methods, biopsies, non-invasive screening and imaging and image-guided biopsies and image-guided corrective surgery.

The present invention provides improved methods for diagnosis and/or detection of a disease, e.g., cancer, by improving sensitivity, the amount of the agent deposited, tissue bioavailability, faster onset of the delivered diagnostic agent. The invention also provides a method for administration of at least one diagnostic agent for the detection of a disease, particularly cancer, comprising delivering the agent into the ID compartment of a subject's skin so that the agent is deposited into the ID compartment and taken up by the lymphatic vasculature.

The formulations, devices and methods of the invention may be particularly useful for methods of integrated diagnosis and therapy. Accurate diagnosis of a disease is largely an unmet need, for example, in oncology, where few diagnostic agents indicate which therapeutic choices will succeed with any reliability.

In some embodiments, this invention also encompasses a kit for assaying an analyte (e.g., a marker) comprising liposome particles containing a detectable agent (e.g., a dye, gas, radioisotope, etc.) and a needle for administration of the particles. The particles may be surface-modified. For example, the particles may be coated with a binder molecule which has affinity for the analyte. Preferably, the signal from the diagnostic agent is sufficiently intense so that the signal may be detected outside the body without a need for surgical procedures. Without being limited by a particular theory, the improved targeting to a specific site achieved by the formulations and methods of the invention may result in the production of more detectable signals.

The present invention demonstrates the synergy gained when intradermal delivery and encapsulation and/or controlled release principles are combined. The synergy is demonstrated with a lipid-based particle or liposome. The invention provides the necessary formulation, device and method detail to rapidly concentrate small and large clinically important molecules. A small molecule example is Sulfa Rhodamine B (“SRB”), a reagent used in diagnostics, and a large molecule example is the humanized monoclonal antibody used in treatment, Herceptin™. Lipsomes containing SRB were used to provide visible evidence of the high tissue concentrations of agent that can be achieved when the novel delivery and particles are combined with intradermal delivery. The inventors demonstrated a commercially available full length antibody molecule could be encapsulated by the same liposome manufacturing process using similar liposome forming components. It was also discovered that such ID delivered liposomes can be monitored from outside the body. In addition, it was discovered that in some embodiments of the invention ID delivered liposomes accumulate in the node and the accumulation can be viewed outside the node. The liposome particles delivered in accordance with the present invention are described by charge, size and capacity for payload, and are further described by their performance characteristics, in-vitro and in-vivo (injection site diffusion, node penetration, suspension stability). The invention further describes greater enhancements in tissue bioavailabilty with particles that transform in-vivo. The invention shows how liposome processing and formulations can be finessed to illuminate different regions and architecture of the lymph node and the patterns can be viewed remotely. The invention further matches particle size with delivery depth to achieve the most efficient intradermal delivery.

3.1 Definitions

As used herein, “intradermal” refers to administration of a biologically active agent into the dermis in such a manner that the agent readily reaches the richly vascularized papillary dermis and is rapidly absorbed into lymphatic vessels. Such can result from placement of the agent in the upper region of the dermis, i.e., the papillary dermis or in the upper portion of the relatively less vascular reticular dermis such that the agent readily diffuses into the papillary dermis. The controlled delivery of a biologically active agent in this dermal compartment below the papillary dermis in the reticular dermis, but sufficiently above the interface between the dermis and the subcutaneous tissue, should enable an efficient (outward) migration of the agent to the (undisturbed) vascular and lymphatic microcapillary bed (in the papillary dermis) without being sequestered in transit by any other cutaneous tissue compartment. In some embodiments, placement of a biologically active agent predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm will result in rapid absorption the agent. Although not intending to be bound by a particular mechanism of action, placement of the biologically active agent predominately at greater depths and/or into the lower portion of the reticular dermis or the SC compartment which results in less effective uptake by the lymphatics.

As used herein, “intradermal delivery” means the delivery of agents to the intradermal compartment as described by Pettis et al. in WO 02/02179 A1 (PCT/US01/20782), U.S. patent application Ser. No. 09/606,909, filed Jun. 29, 2000, and U.S. Publication No. 2005-0163711 A1, published Jul. 28, 2005; each of which is incorporated herein by reference in their entireties.

As used herein, “ID Mantoux delivery” refers to the traditional ID Mantoux tuberculin test where an agent is injected at a shallow angle to the skin surface using a 27 or 30 gauge needle and standard syringe (see, e.g., Flynn et al., Chest 106: 1463-5, 1994, which is incorporated herein by reference in its entirety). The Mantoux technique involves inserting the needle into the skin laterally, then “snaking” the needle further into the ID tissue. The technique is known to be quite difficult to perform and requires specialized training. A degree of imprecision in placement of the delivery results in a significant number of false negative test results. Moreover, the method involves a localized injection to elicit a response at the site of injection and the Mantoux approach has not led to the use of intradermal injection for systemic administration of agents. When delivering the agent by ID Mantoux, the needle is substantially parallel to the surface at the skin, preferably at an angle of no more that 30° and best described as being between 10° and 15°. Mantoux deposition of injectate, when performed correctly, results in an elliptical pattern with the injectate in the SC and ID tissues. ID deposition as described herein results in a rounded deposition pattern of the injectate contained in the ID tissue. When delivering an agent by the ID Mantoux method, the insertion angle of the needle is preferably at a 15° angle parallel to the skin surface. Histological examination of the injection site after an agent has been administered by ID Mantoux results in an elliptical wheal deposition pattern. ID Mantoux method is typically used clinically in diagnostic procedures such as sentinal node biopsy procedures for detection of tumors, however the method is quite difficult to perform and requires specialized training and has numerous limitations including, sites of administration, complications of injection, and patient discomfort.

As used herein, subcutaneous delivery refers to deposition of an agent into the subcutaneous layer of a subject's skin at a depth greater than 2.5 mm.

As used herein, “pharmacokinetics, pharmacodynamics and bioavailability” are as described by Pettis et al. in WO 02/02179 A1 (PCT/US01/20782 having a priority date of Jun. 29, 2000, and U.S. Publication No 2005-0163711 A1, published Jul. 28, 2005).

As used herein, the term “particles containing” a biologically active agent (e.g., diagnostic or therapeutic agent) refers to any encapsulated and/or controlled release biologically active agents including, but not limited to, a sac (e.g., liposome) or a microcapsule encapsulating a biologically active agent. Lipid based particles include any liposome based particles that can be used to encapsulate and/or controlled release biologically active agents. Examples include, but are not limited to, liposomes, lipid vesicles, microbubbles, microballoons, LUVs, MLVs, LMVs and SUVs. As used herein, the terms “particulate agents” and “biologically active particulate agents” include but are not limited to liposomes as well as non-lipid based particles.

As used herein “tissue” refers to a group or layer of cells that together perform a function including but not limited to skin or lymphatic tissue (e.g., lymph nodes).

As used herein, “tissue-bioavailability” means the amount of an agent that is biologically available in vivo in a particular tissue. These amounts are commonly measured as activities that may relate to binding, labeling, detection, transport, stability, biological effect, or other measurable properties useful for diagnosis and/or therapy. In addition, it is understood that the definition of “tissue-bioavailability” also includes the amount of an agent available for use in a particular tissue. “Tissue-bioavailability” includes the total amount of the agent accumulated in a particular tissue, the amount of the agent presented to the particular tissue, the amount of the agent accumulated per mass/volume of particular tissue, and amount of the agent accumulated per unit time in a particular mass/volume of the particular tissue. Tissue bioavailability includes the amount of an agent that is available in vivo in a particular tissue.

As used herein, “conventional delivery” means any method for delivering any material that has, or is thought to have, improved biological kinetics and biological dynamics similar to, or slower than, subcutaneous delivery. Conventional delivery may include subcutaneous, iontophoretic, and intradermal delivery methods such as those described in U.S. Pat. No. 5,800,420 to Gross.

Targeted tissues include, but are not limited to, the intradermal compartment near the site of administration and the local lymphatic structures including initial lymphatics, lymphatic vessels, ducts and lymph nodes. Targeted tissues also include but are not limited to, skin tissue, and lymphatic tissue (e.g., lymph nodes), particularly lymph tissue containing diseased or cancer cells.

As used herein, a “diagnostic or therapeutic agent” refers to any molecules that are used for the purpose of diagnosis or therapy of a disease or disorder. Diagnostic agents may include compounds such as, but not limited to, dyes, gas, metals, or radioisotopes. Therapeutic agents may include compounds such as, but not limited to, proteins, immunoglobulins (e.g., multi-specific Igs, single chain Igs, Ig fragments, polyclonal antibodies and their fragments, monoclonal antibodies and their fragments), peptides (e.g., peptide receptors, selectins), binding proteins, chemospecific agents (e.g., cyptands, crown ethers, boronates), chemotoxic agents (e.g., anti-cancer agents), and small molecule drugs. In regards to diagnostics, the particle is sometimes referred to as a particulate label and containing a detector molecule.

As used herein, “chemospecific agent” means a chemically synthesized molecule that binds specifically to a bio-molecule. Examples of chemospecific agents include, but are not limited to, PNAs such as GeneGRIP™ as commercialized by Gene Therapy Systems Inc., photoaptamers as commercialized by SomaLogic, sialic acid binders as described by Shinkai, S, et. al. J. A. Chem. Soc. 2001, 123. 10239-10244, Wang et al., Current Organic Chemistry 2002, 6, 1285-1317, Striegler, S. Current Organic Chemistry 2003, 7, 81-102, Wang, et. al., Bioorganic & Medicinal Chemistry Letters 2002, 2175-2177, and boronic acids for detection of carbohydrates as described in US 2002/0143475 (Colorimetric and Fluorometric analysis of Carbohydrates). All of the above mentioned references are incorporated herein by reference in their entireties.

As used herein, “chemotoxic agents” refer to drugs with cytotoxic activity. A typical example is anti-cancer drugs. Specific examples include, but are not limited to, anthracyclines, taxols, and platinum-based drugs, including, but not limited to, cisplatin, carboplatin, doxorubicin, and paclitaxel. Chemotoxic agents also include various anti-bacterial, anti-viral or anti-fungal (e.g., amphotericin) agents. The primary objective for many chemotoxic agents is to achieve a favorable “drug ratio” or “index” between the healthy tissue and the diseased tissue. Thus, an objective is to achieve a high concentration of the chemotoxic agent in the diseased tissue and less in the healthy tissue, e.g. a high concentration of chemotoxic agent in the lymph system where cancer spreads and low concentrations of the chemotoxic agent in the central organs. The expression may be “lymph to organ index” or “lymph to blood index.” More specifically, the expression may be “lymph node to organ index,” “lymph node to kidney,” “lymph node to spleen,” etc.

As used herein, “marker” means any receptor, molecule or other chemical or biological entity that is specifically found in tissue that it is desired to identify, in particular tissue affected by a disease or disorder (e.g. a metastases). Where an antibody is used as the tracer, the marker is an antigen. Examples of antigen markers include CD4, CD8, CD90 and other antigenic markers mentioned herein, as well as those that are known in the art. Non-limiting examples of such markers include: proteins or receptors such as Her2/neu or epidermal growth factor receptor (EGFR) for breast cancer, melastatin for melanoma, CD22 for lymphoma, and HIV protease for HIV infection. Markers may also be carbohydrates such as sialic acids for metastases or NCAMs for neuroendocrine disease or cancer. An example of such a disease is Diffuse Large B Cell lymphoma. The term “marker” may be used interchangeably with the term “analyte,” in particular, in connection with the diagnostic methods and formulations of the invention.

As used herein, the terms “binder molecule,” “binder agent,” “binder”, and “bridging agent” refer to any molecule which has affinity for another molecule. A binder may be carbohydrate, lipid, protein or nucleic acid or certain hybrid molecule containing more than one type of structure such as a glycoprotein contains protein and carbohydrate. A binder may also be hapten, vitamin, metal or steroid. A binder may be a carbohydrate binding protein such as maltose binding protein (MBP) or glucose galactose binding protein (GGBP). A binder may be a DNA binding protein. A binder may be antibody or a fragment thereof. The antibody may be polyclonal, monoclonal, native, recombinant, humanized, chimeric or single chain. The antibody may be of any class or species. A binder may be peptide or peptide mimic. The molecules described above may represent one or both of the molecules in a binder set. For example, a binder set may represent two antibodies, wherein one has specificity for the other. A binder set may be biotin and avidin as exemplified herein. A binder set may involve biotin and an antibody specific for biotin. A binder set may involve immobilizing one of two agents on the surface of the particle, whereas the surface linked binder could be an immunogen such as the influenza hemaglutinin and the corresponding binder is an antibody specific for the hemaglutinin that is free in solution. For liposomal particles, one of the binders may be surface attached using any number derivatized lipids available from Avanti Lipids Incorporated or Northern Lipids Incorporated using methods known in the art. In some embodiments, the term “bridging agent” may refer to molecules which have affinities for one or more molecules, and thus, can be used to bring two or more such molecules together in close proximity.

As used herein, the terms “disorder” and “disease” are used interchangeably to refer to a condition in a subject. Diseases include to any interruption, cessation, or disorder of body functions, systems or organs. Diseases may include any disturbance of the lymphatic system.

As used herein, the term “cancer” refers to a neoplasm or tumor resulting from abnormal uncontrolled growth of cells. As used herein, cancer explicitly includes, leukemias and lymphomas. The term “cancer” refers to a disease involving cells that have the potential to metastasize to distal sites and exhibit phenotypic traits that differ from those of non-cancer cells, for example, formation of colonies in a three-dimensional substrate such as soft agar or the formation of tubular networks or web like matrices in a three-dimensional basement membrane or extracellular matrix preparation. Non-cancer cells do not form colonies in soft agar and form distinct sphere-like structures in three-dimensional basement membrane or extracellular matrix preparations. Cancer cells acquire a characteristic set of ftunctional capabilities during their development, albeit through various mechanisms. Such capabilities include evading apoptosis, self-sufficiency in growth signals, insensitivity to anti-growth signals, tissue invasion/metastasis, limitless explicative potential, and sustained angiogenesis. The term “cancer cell” is meant to encompass both pre-malignant and malignant cancer cells. In some embodiments, cancer refers to a benign tumor, which has remained localized. In other embodiments, cancer refers to a malignant tumor, which has invaded and destroyed neighboring body structures and spread to distant sites. In yet other embodiments, the cancer is associated with a specific cancer antigen.

As used herein, the terms “subject” and “patient” are used interchangeably. As used herein, a subject is preferably a mammal such as a non-primate (e.g., cows, pigs, horses, cats, dogs, rats etc.) and a primate (e.g., monkey and human), most preferably a human.

### 4. BRIEF DESCRIPTION OF FIGURES

FIG. 1 provides an illustrated example of the steps of particle transformation in vivo. 1. Small carrier particles with label (e.g. biotin) penetrate capillary wall cell junctions with high efficiency. 2. Small particles encounter linking agent (e.g. avidin) in the capillary and transform into larger particle aggregates. 3. Aggregated particles carrying more agent are physically trapped by nodal network. Free particles pass through.

FIG. 2 illustrates liposomal particles according to their size and charge penetrating a lymph node after ID delivery using a 1 mm×34 gauge needle.

FIG. 3 shows a summary of particle properties and their performance with regard to node penetration, payload, solution properties, and delivery site observations.

FIG. 4 shows the levels of nodal platinum following administration of encapsulated platinum ID and IV, and free platinum ID and IV.

FIGS. 5A-5D show the levels of platinum in various organs following administration of encapsulated platinum ID and IV, and free platinum ID and IV (5A: heart; 5B: kidney; 5C: liver; and 5D: lung).

FIG. 6 shows the effects of the administration of decoy particles prior to the administration of carboplatin containing particles, i.e. the use of decoy particles to reduce the amount of chemotoxic drug accumulation in organs.

FIG. 7 shows the levels of nodal HERCEPTIN® in lymph node following administration of encapsulated platinum ID and IV, and free platinum ID and IV.

FIG. 8 shows an exploded, perspective illustration of a needle assembly design according to this invention

FIG. 9 shows a partial cross-sectional illustration of the embodiment in FIG. 8.

FIG. 10 illustrates the embodiment of FIG. 8 attached to a syringe body to form an injection device.

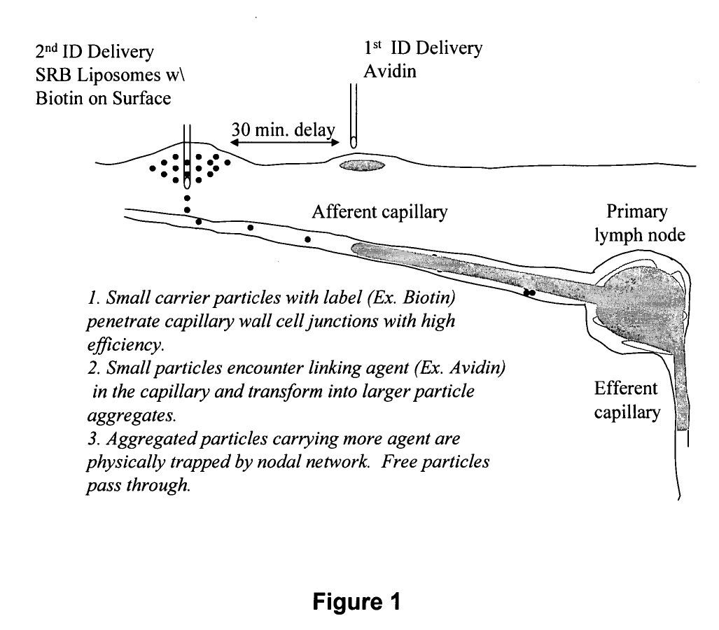

### 5. DETAILED DESCRIPTION OF THE INVENTION

In one embodiment, the present invention encompasses a method for administering one or more biologically active agents to a subject's skin, in which the biologically active agent in particulate form (e.g., encapsulated within particles) is delivered to the intradermal (ID) compartment of the subject's skin in a condition that causes the agents to form an aggregate. Subsequent to the delivery, during their movement to the lymph node, the agents form an aggregate sufficiently large to be retained in the node. In another embodiment, this invention also encompasses a method for administering one or more biologically active agents to a subject's skin, in which the biologically active agent in particulate form (e.g., encapsulated within particles, preferably liposomes) is delivered to the intradermal (ID) compartment of the subject's skin. It was discovered that combining the ID delivery with specific size, charge, and/or loading capacity of liposomes can result in the retainment of the liposome particles in a targeted area of patient's body (e.g., lymphatic tissue). In other embodiments, the present invention also encompasses compositions comprising the particles containing a biologically active agent, and kits containing the formulations.

The present invention is based, in part, on the unexpected discovery by the inventors that when such agents are delivered to the ID compartment using methods of the invention, they are better retained in lymphatic tissue (e.g., lymph node) compared to conventional modes of delivery, including subcutaneous and intravenous deliveries, and thus provide the benefits disclosed herein. Although not intending to be bound by a particular mechanism of action, ID delivery of the agents using methods described herein is believed to better facilitate the aggregation of the agents than conventional deliveries (e.g., subcutaneous and intravenous), which results in a higher degree of accumulation of the agents in the lymph node than conventional deliveries.

The present invention is also based, in part, on the unexpected discovery by the inventors that when such agents are delivered to the ID compartment using methods of this invention, they are transported to the local lymphatic system rapidly as compared to conventional modes of delivery, including subcutaneous delivery and intravenous delivery, and thus provide the benefits disclosed herein. Although not intending to be bound by a particular mechanism of action, agents delivered in accordance with the methods of the invention are transported in vivo through the local lymphatic system. Although not intending to be bound by a particular mechanism of action, it is believed that the biomechanical manipulation of the extracellular matrix (ECM) through the precise delivery of agents in the intradermal compartment enables rapid uptake into the local lymphatics and lymph nodes by the methods described herein.

The present invention may provide, among other benefits, rapid uptake into the local lymphatics, improved targeting to a particular tissue, i.e., improved deposition of the delivered agent into the particular tissue, i.e., group or layer of cells that together perform a specific function, improved systemic bioavailability, improved tissue bioavailability, improved deposition of a pre-selected volume of the agent to be administered, improved tissue-specific kinetics. Such benefits of the invention are improved over other methods of delivering biologically active agents which deposit the agent into deeper tissue compartments than the intradermal compartment including for example subcutaneous compartment and intravenous injection. Such benefits of the methods of the invention may be especially useful for the delivery of diagnostic and therapeutic agents. Intradermal delivery of a biologically active agent in accordance with the formulations, devices and methods of the invention preferably deposits the agent into the intradermal and lymphatic compartments, thus creating a rapid and biologically significant concentration of the diagnostic agent in these compartments. Rapid diagnostics may therefore be performed using less diagnostic agent with significant advantages as outlined herein. In the case of the delivery of therapeutic agents, the advantages preferably include the reduction of toxic therapeutic agents and toxic side affects; accordingly, higher concentrations of drug can be administered than previously tolerated and thereby improve therapeutic outcomes.

Intradermally delivered biologically active agents preferably have improved tissue bioavailability in a particular tissue, including but not limited to, skin tissue, lymphatic tissue (e.g., lymph nodes). The delivery of a biologically active agent in accordance with the methods of the invention results in improved tissue bioavailability as compared to when the same agent is delivered to the subcutaneous (SC) compartment or when the same agent is delivered by the intravenous method. Improved tissue bioavailability of agents delivered in accordance with the methods of the invention may be particularly useful when delivering diagnostic agents to the ID compartment, as it reduces the amount of the diagnostic and therapeutic agent required for each diagnostic or treatment procedure, which may be difficult and costly to obtain. The reduced amount of the diagnostic and therapeutic agent further reduces the likelihood of side effects associated with the procedure, e.g., toxicity.

The improved tissue bioavailability of the agents delivered in accordance with the methods of the invention can be determined using methods and parameters known to those skilled in the art, for example, by measuring the total amount of the agent accumulated in a particular tissue using, for example, histological methods known to those skilled in the art and disclosed herein. Alternatively, improved tissue bioavailability of the agents can be assessed as the amount of the agent presented to the particular tissue, the amount of the agent accumulated per mass or volume of a particular tissue, amount of the agent accumulated per unit time in a particular mass or volume of a particular tissue.

Without being limited by a particular theory, encapsulated and/or controlled release versions of biologically active agents delivered in accordance with the methods of the invention are deposited in the intradermal compartment and first distributed with high bioavailability to the lymphatic tissue local to the administration site, followed by a more wide spread lymphatic delivery. In some embodiments, the methods of the present invention are particularly effective for diagnosis and treatment of a disease or disorder.

Intradermally delivered encapsulated and/or controlled release formulations of biologically active agents, especially diagnostic and therapeutic agents, preferably exhibit more rapid onset versus conventional delivery including SC delivery. The methods of the invention thus may confer several advantages when delivering an encapsulated and/or controlled release agents to the ID compartment of a subject's skin. Potential benefits include, but are not limited to: 1) the methods disclosed herein reduce potential side effects and discomfort due to the diagnostic or therapeutic procedures; 2) they enable the rapid and repeated trial of sequential procedures in a single diagnostic or treatment session; 3) they reduce the time required in expensive medical or imaging facilities; 4) they facilitate real time studies of physiology; 5) they reduce potential background signal generated by unbound and un-cleared diagnostic reagents; and 6) patients experience reduced pain from the methods of the invention in comparison to pain perceived from IV administration, SC injection, Mantoux injection, or surgical biopsy.

In some embodiments, delivering encapsulated and/or controlled release formulations of biologically active agents, including diagnostic and therapeutic agents, in accordance with the methods of the invention is preferred over traditional modes of delivery including SC delivery and intravenous delivery because the amount of the pre-selected dose of the agent deposited in the lymphatic tissue is increased, as measured, for example, using histopathological methods or other methods known to one skilled in the art, such as ELISA and imaging methods disclosed herein.

As used herein, delivery to the intradermal compartment or intradermal delivery is intended to mean administration of a biologically active agent into the dermis in such a manner that the agent readily reaches the richly vascularized papillary dermis and is rapidly absorbed into lymphatic vessels. Such can result from placement of the agent in the upper region of the dermis, i.e., the papillary dermis or in the upper portion of the relatively less vascular reticular dermis such that the agent readily diffuses into the papillary dermis. The particles containing a biologically active agent in this dermal compartment below the papillary dermis in the reticular dermis, but sufficiently above the interface between the dermis and the subcutaneous tissue, should enable an efficient (outward) migration of the agent to the (undisturbed) vascular and lymphatic microcapillary bed (in the papillary dermis). In some embodiments, placement of a biologically active agent predominately at a depth of at least about 0.3 mm, more preferably, at least about 0.4 mm and most preferably at least about 0.5 mm up to a depth of no more than about 2.5 mm, more preferably, no more than about 2.0 mm and most preferably no more than about 1.7 mm result in rapid absorption of the agent. Although not intending to be bound by a particular mechanism of action, placement of the biologically active agent predominately at greater depths and/or into the lower portion of the reticular dermis may result in less effective uptake of the agent by the lymphatics, as the agent will be slowly absorbed in the less vascular reticular dermis or in the subcutaneous compartment. For lipid-based controlled release agents, preferred delivery depth is from about 0.3 mm to about 1.25 mm, preferably about 1 mm.

In some embodiments, particulate (e.g., encapsulated) biologically active agents, including diagnostic and therapeutic agents, delivered in accordance with the methods of the invention achieve higher maximum concentrations of the agents and allow reduced overall dosing. Therefore, the dose can be reduced, providing an economic benefit, as well as a physiological benefit since lesser amounts of the drug or diagnostic agent has to be cleared by the body.

The improved benefits associated with ID delivery of encapsulated and/or controlled release biologically active agents in accordance with the methods of the invention can be achieved using not only microdevice-based injection systems. In specific embodiments, the administration of the biologically active agent is accomplished through insertion of a needle or cannula into the intradermal compartment of the subject's skin.

The intradermal delivery of diagnostic and therapeutic agents in accordance with the present invention may be particularly beneficial in the diagnosis and treatment of diseases, including chronic and acute diseases, which include, but are not limited to, lymphoma, breast cancer, melanoma, colorectal cancer, head and neck cancer, lung cancer, cancer metastasis, including micrometastasis, viral infections, e.g., HIV, diseases or disorders of the lymphatic system, any disease affecting the lymph nodes. Although not intending to be bound by a particular mechanism of action, diagnostic agents delivered in accordance with the methods of the invention are deposited in the intradermal compartment and taken up by the lymphatic system, where its recognition and binding of a particular cell in a particular tissue indicate the presence of a cell or disease state. The present invention is also useful for diagnostic procedures including, but not limited to, surgical methods, biopsies, non-invasive screening and imaging and image-guided biopsies.

In some embodiments, the present invention provides improved methods for diagnosis and/or detection of a disease, e.g., cancer, by improving sensitivity, the amount of the agent deposited, tissue bioavailability, faster onset of the delivered diagnostic agent. The invention provides a method for administration of at least one diagnostic agent in a particulate from for the detection of a disease, such as cancer, comprising delivering the agent into the ID compartment of a subject's skin so that the agent is deposited into the ID compartment and taken up by the lymphatic vasculature.

The formulations, devices and methods of the invention may also be particularly useful for methods of integrated diagnosis and therapy. Accurate diagnosis of a disease is largely an unmet need for example in oncology, where few diagnostic agents indicate which therapeutic choices will succeed with any reliability.

The present invention is based, in part, on the synergy gained when intradermal delivery and encapsulation and/or controlled release principles and/or drug in particulate form are combined. The synergy is demonstrated with a lipid-based particle or liposome. The invention encompasses formulations, devices and methods of rapidly concentrating small and large clinically important molecules. An exemplary small molecule used in specific embodiments of the invention is Sulfa Rhodamine B (“SRB”), a reagent used in diagnostics. An exemplary large molecule used in specific embodiments of the invention is the humanized monoclonal antibody used in treatment, Herceptin™. Liposomes containing SRB were used to provide visible evidence of the high tissue concentrations of agent that can be achieved when the novel delivery and particles are combined with intradermal delivery. The inventors demonstrated a commercially available full length antibody molecule could be encapsulated by the same liposome manufacturing process using similar liposome forming components. It was also discovered that such ID delivered liposomes can be monitored from outside the body, which was achieved by substituting the SRB dye with Indocyanine Green (IGC or Cardiogreen). The invention provides the description of the liposome particles by charge, size and payload. The liposome particles are further described by their performance characteristics, in-vitro and in-vivo (injection site diffusion, node penetration, suspension stability). The invention further describes greater enhancements in tissue bioavailabilty with particles that transform in-vivo. The invention shows how liposome processing and formulations can be finessed to illuminate different regions and architecture of the lymph node and the patterns can be viewed remotely. The invention further matches particle size with delivery depth to achieve the most efficient intradermal delivery.

In one embodiment, this invention encompasses a method of delivering a diagnostic or therapeutic agent to lymphatic tissue of a subject comprising delivering particles containing said agent to the intradermal compartment of the subject's skin, introducing to the subject a condition that causes the particles to aggregate subsequent to the delivery of said particles to the intradermal compartment, wherein said aggregates are of sufficient size to be retained by lymphatic tissue. In one embodiment, the particle is a sac (e.g., liposome) or a microcapsule.

In one embodiment, the aggregate is formed using surface modified liposomes. In another embodiment, the surface-modification include coating of liposome surface with a binder molecule. An example is biotinylated liposomes, administered in combination with avidin. Without being limited by a particular theory, it is believed that avidin-biotin interaction causes the biotinylated liposomes containing the agent to aggregate to form an aggregate sufficiently large to be retained by lymphatic tissue (e.g., lymph node).

In one specific embodiment, avidin is administered intradermally prior to the intradermal administration of biotinylated liposomes. In another specific embodiment, avidin is administered about 30 minutes prior to the administration of biotinylated liposomes. In another embodiment, avidin is administered at a site from 0 to about 5 cm apart, preferably from about 0.5 to about 3 cm apart, preferably about 1 cm apart, from the site of liposome administration. In one specific embodiment, avidin is administered about 30 minutes prior to the administration of biotinylated liposomes, at a site about 1 cm apart from the site of liposome administration. Although the invention is described herein using a particular example, i.e., biotinylated liposome, in combination with avidin, this invention encompasses other variations of the method known to those of ordinary skill in the art as long as the method is based on intradermal delivery of the agents that can be transformed (e.g. aggregated) subsequent to the delivery. For example, any liposomes such as those comprising cholesterol and at least one of DSPA, DSPC, DSPG, DSPE, HSPC, DSPE-MPEG2000, DSPE-PEG350, DPPG, DOPC, sphingomyelin, dihydrosphingomyelin or Diol-Biotin, and optionally one of alpha tocopherol or triolein, may be used.

Further, instead of liposome based particles, other lipid based particles, as described herein, may be used as well as lipid-based emulsions. In addition, other non-lipid based particles may be used that fit the charge and size and payload capacity specified for intradermal delivery and lymphatic application, e.g. −1 to −80 mV for zeta potential and 10 to 300 nm for diameter and payload capacity, such as, but not limited to, gold based particles or dendrimers or nanoshells or quantum dots. Microspheres formed with albumin, silicon and PLGA may also be used. Likewise, other binding agents or a pair of molecules that exhibit specific interaction to each other may be used for the surface modification. Furthermore, commercially available liposome formulations such as, but not limited to, Ambisone™ and SonoVue™ and like formulations may be used, and are preferably modified to meet the preferable zeta-potential (charge), size and payload capacity described herein.

Aggregation of the particles may be achieved using non-specific fusion agents delivered free in solution. Examples include, but are not limited to, use of a condensing agent such as polyethyleneglycol. Aggregation of the particles may also be achieved using a non-chemical means that causes fusion of the particles. Examples include, but are not limited to, sonowaves, magnetic force, and heat from a laser.

In one embodiment, the aggregate formed from the particles may be targeted to a specific type of cells. For example, the aggregate may contain a second label (e.g., antibody) that would anchor the aggregate to specific type of cells by targeting the cell markers (e.g., cancer cell markers).

In another embodiment, liposomes without surface-modification may be delivered. In some embodiments, adjusting the size, charge, and/or loading capacity of liposome particles enables the particles to be retained by lymphatic tissue when delivered ID. In one embodiment, the liposome particles have a size of about 10 to 300 nm, a zeta-potential of about −1 to −80 mV, and/or contain 0.8 to 20 molar percent of a biologically active agent.

In one embodiment, this invention encompasses compositions for intradermal administration comprising surface-modified liposome particles, wherein the liposome particles contain a diagnostic or therapeutic agent, to improve the therapeutic or diagnostic characteristics of the active agent, and methods of intradermally delivering the formulations to a subject. Surface-modification and the use of binder molecules are as described in connection with methods of the invention.

In another embodiment, this invention also encompasses compositions for intradermal administration comprising liposome particles, without surface-modification, containing a diagnostic or therapeutic agent, wherein said liposome particles have specific size, charge, and/or loading capacity. In one embodiment, the liposome particles have a size of about 10 to 300 nm (nanometer), a zeta-potential of about −1 to −80 mV, and/or contain 0.8 to 20 molar percent of a biologically active agent. In another preferred embodiment the liposome particles have a size of about 50 to 250 nm, a zeta-potential value ranging from −30 to −50 mV, and on a mole-to-mole basis 10-20% of the particles are made up of active ingredient and the other 80-90% of the particles are made up of molecules that encapsulate or entrap the active ingredient.

In all methods and formulations of the present invention, liposome particles of a broad range of size, charge, payload (active agent to Pi ratio, as determined using the methods described herein), and/or constituents may be used. In one embodiment, liposome particles having a size of from about 10 nm to about 17,000 nm, from about 10 nm to about 6450 nm, from about 10 nm to about 2310 nm, from about 10 nm to about 770 nm, or from about 10 nm to about 300 nm, as determined using the methods described herein, are used in connection with the formulations and methods of the invention.

In another embodiment, liposome particles having a charge of from about −1 mV zeta potential to about −80 mV zetapotential, from about −10 mV to about −80 mV, from about −10 mV to about −70 mV, from about −15 mV to about −60 mV, from about −20 mV to about −60 mV, from about −10 mV to about −40 mV, from about −30 mV to about −50 mV, from about −20 mV to about −40 mV, or form about −60 mV to about −80 mV, as determined using the methods described herein, are used in connection with the formulations and methods of the invention. In particular embodiments, liposome particles having a charge of about −1 mV to about −20 mV or about −60 mV to about −17 mV may be used.

In one embodiment, the entrapped, encapsulated or captured active agent used in connection with the formulations and methods of the invention may have a molecular weight from about 300 to 158,000. A diagnostic or therapeutic agent may represent from 0.8% to 20% of the particle on a molar basis and 0.07 to 7% on a per weight basis. More particular to diagnostics, the particle is sometimes referred to as a particulate label and contains a detector molecule. The diagnostic or detector agent may comprise 0.8% of the particle on a molar basis, preferably 4% and most preferred is 20%. The broad range in composition can be explained by the wide variety of detector agents available, being of broad solubility and molecular weight. Low molecular weight highly soluble dyes such as Sulfa Rhodamine-B will often provide the best reportive value when present in the particle at or near 20% of the total particle formula (on a molar basis) as used within the SRB-liposomes studies. Lipids constitute˜80% of the average liposome used in these studies and the other 20% is dye, on a molar basis. In contrast, higher molecular weight less soluble detectors may only be packaged at the low end of the scale, narrowly achieving the recommended range, 0.8% on a molar basis. In some instances, based on detector attributes and instrumentation used for analysis, a detector may only be required on the particle surface, where as less detector is needed per particle. The percentages provided above for the detector portion are particularly relevant for particles having a diameter of about 0.1 to 1000 nm, about 1 to 500 nm, about 5 to 200 nm, about 10 to 500 nm, about 1 to 100, about 10 to 100, and about 10 to 300 nm. In one embodiment, the preferred size is from about 10 nm to about 300 nm. Accordingly ranges for a low molecular weight highly soluble detector may span 4 to 20%. The range for a high molecular weigh poorly soluble detector may span 0.8 to 4% on a molar basis.

More particular to the treatment, a therapeutic agent may represent 0.07% of the particle on a per weight basis, potentially 0.7% and at times 7% is preferred when the drug is a low molecular weight such as a chemotoxic substance used in the treatment of cancer. Poor solubility or high molecular weight can restrict packaging. The high nodal values of Herceptin™ (high molecular weight/moderate solubility) demonstrated within the invention can be achieved with a particle being 0.07% antibody on a per weight basis. The high nodal values of carboplatin (low molecular weight/moderate solubility) can be achieved with a particle being 7% carboplatin on a per weight basis. The percentages provided above for the drug portion are particularly relevant for particles having a diameter of 300-10 nm. Accordingly, a functional particulate for treatment comprising a high molecular weight protein drug (ex. antibody) should contain 0.07 to 0.7% of the therapeutic agent on a per weight basis. A functional particulate for treatment comprising a low molecular weight (ex. carboplatin) should contain 0.7 to 7% of the therapeutic agent on a per weigh basis. These parameters are relevant for particles tailored for intradermal delivery and lymphatic application. The parameters are particularly relevant for liposome particles tailored for intradermal delivery and lymphatic application. Described by ratio, enabling liposome particles have a captured detector to lipid ratio (on a molar basis) of ˜1:5 to 1:125 and captured drug to lipid ratio (on a per weight basis) of ˜1:14 to 1:1400.

In one embodiment, liposomes comprising cholesterol and at least one of DSPA, DSPC, DSPG, DSPE, HSPC, DSPE-PEG 2000, DSPE-PEG 750, DSPE-PEG 350, DPPG, DOPC, sphingomyelin, dihydrosphingomyelin or Diol-Biotin, and optionally one of alpha tocopherol or triolein, may be used in connection with methods and formulations of the invention. As used herein, DSPC is 1,2-Distearoyl-sn-Glycero-3-Phosphocholine; DSPG is 1,2-Distearoyl-sn-Glycero-3-[Phospho-rac-(1-glycerol)] (Sodium Salt); DSPA is 1,2-Distearoyl-sn-Glycero-30-Phosphate (Monosodium Salt); DSPE is 1,2-Distearoyl-sn-Glycero-3-Phosphoethanolamine; HSPC is L-α-Phosphatidylcholine- Hydrogenated (Soy Origin); SM is sphingomyelin (egg or chicken origin); DPPG is 1,2-Dipalmitoyl-sn-Glycero-3-[Phospho-rac-(1-glycerol)] (Sodium Salt); DOPC is 1,2-Dioleoyl-sn-Glycero-3-Phosphocholine; triolein is glyceryl trioleate; DSPE PEG 350 is 1,2-Distearoyl-sn-Glycero-3-Phosphoethanolamine-N-[Methoxy(Polyethylene glycol)-350] (Ammonium Salt); DSPE PEG 750 is 1,2-Distearoyl-sn-Glycero-3-Phosphoethanolamine-N-[Methoxy(Polyethylene glycol)-750] (Ammonium Salt); and DSPE-PEG 2000 is 1,2-Distearoyl-sn-Glycero-3-Phosphoethanolamine-N-[Methoxy(Polyethylene glycol)-2000] (Ammonium Salt), Diol-Biotin is 1,2-Dioleoyl-sn-Glycero-3-Phosphoethanolamine-N-(Biotinyl) (Sodium Salt).

In one embodiment, the encapsulated active agent used in connection with the formulations and methods of the invention may have a molecular weight of from about 500, more specifically 586, to about 158,000. In another embodiment, liposome particles having a payload of from about 0.001 nmoles to about 25 nmoles per 50 nmoles phosphate, 0.005 nmoles to about 20 nmoles per 50 nmoles phosphate, 0.01 nmoles to about 15 nmoles per 50 nmoles phosphate, from about 0.015 nmoles to about 12 nmoles per 50 nmoles phosphate, 0.02 nmoles to about 11 nmoles per 50 nmoles phosphate, from about 0.024 nmoles to about 10.956 nmoles per 50 nmoles phosphate, as determined using the methods described herein, are used in connection with the formulations and methods of the invention.

In methods of the present invention, a wide range of amount of the particles containing a biologically active agent may be administered. For example, the particles may be administered in an amount of about 0.01 to 50, about 0.1 to 40, about 1 to 25, about 5 to 30, about 1 to 10, about 10 to 25, about 1 to 24, or about 12 to 24 μmole/ml phosphorus. Similarly, in formulations of the present invention, the particles may be present in an amount of 0.01 to 50, about 0.1 to 40, about 1 to 25, about 5 to 30, about 1 to 10, about 10 to 25, about 1 to 24, or about 12 to 24 μmole/ml phosphorus.

A wide variety of active agents may be used in connection with the formulations and methods of the invention. Examples include, but are not limited to, a radionucleotide, a gas, a dye (e.g., NIR dye such as ICG, and SRB), an antibody (e.g., those against cancer antigen), a cytokine, and a chemotoxic agent (e.g., anti-cancer agents such as doxorubicin, cisplatin, carboplatin, and paclitaxel), as well as those others described herein elsewhere.

The formulations of the invention, once delivered to a subject, preferably can be tracked remotely with naked eyes. In some embodiments, the formulations of the invention may be tracked remotely using methods known in the art (e.g., use of instrumentation), as well as those described herein. The particles in the encapsulated or controlled-release formulations of the invention, once in vivo, undergo a transformation to allow a better retention in the lymph nodes or other targeted tissue. In one embodiment, the transformation is change in shape. In another embodiment, the transformation is a change in outer diameter obtained through, for example, aggregation of the particles. The transformation can occur through aggregation or fusion of one or more particles. It is not necessary that all particles administered according to methods of the invention, or present in formulations of the invention, undergo a transformation to provide enhanced benefits.

In one embodiment, the formulations of the invention may be administered according to methods of the invention using a 30-34 g needle of about 1 to 2 mm in length. Other types of needles and devices that may be used in connection with methods of the invention are disclosed in detail herein elsewhere.

In one embodiment, this invention also encompasses a kit for diagnostic or therapeutic uses comprising formulations and devices of the invention described herein.

In some embodiments, the present invention provides unique advantages by combining intradermal delivery with payload optimization, which is achieved by encapsulation of the agents. As the individual particles undergoes transformation (e.g., aggregation and fusion) subsequent to the delivery, the particles are initially small enough to traverse the interstitial tissue, and subsequently, the aggregated particles are large enough for physical retention in the node and advantageous for concentrating signal around a particular structure in the node. The resulting aggregate carries sufficiently high payload to meet the minimum signal required by conventional clinical imaging devices. Furthermore, larger particles deliver higher doses of the agents, or can be manipulated to release the agent over a greater period of time (controlled release).

The delivery of a biologically active agent in accordance with the methods of the invention allows for improved accumulation of the agents in lymph nodes as compared to when the same agent is delivered to the subcutaneous (SC) or intravenous (IV) compartment. Improved accumulation of agents in lymph node delivered in accordance with the methods of the invention is particularly useful, for example, when diagnostic or therapeutic agents are to be targeted specifically to the lymph nodes, as the methods of the invention reduces the need for the high concentration of the agents when they are locally applied to lymph nodes. The reduced amount of the agent further reduces the likelihood of side effects associated with the agent, e.g., toxicity.

Biologically active agents delivered in accordance with the methods of the invention are deposited in the intradermal compartment and first distributed with high bioavailability to the lymphatic tissue local to the administration site. During the movement, it is believed that particles comprising the agents form an aggregate sufficiently large to prevent the agents from passing completely through the lymph node, so that accumulation of the agents in the lymph node can be achieved. In some embodiments, the methods of the present invention are particularly effective for diagnosis or treatment of a disease, disorder, or infection wherein targeting of biologically agents to the lymph node is desired, for example, localized metastases in lymph node.

In some embodiments, biologically active agents, including diagnostic or therapeutic agents delivered in accordance with the methods of the invention are specifically accumulated in the lymph nodes. In other embodiments, biologically active agents delivered in accordance with the methods of the invention are delivered to the ID compartment so that the amount of the pre-selected dose of the agent deposited in the lymph node is increased by at least 0.1% compared to when the agent is delivered outside of the intradermal space, e.g., subcutaneous compartment (SC) or intravenous compartment (IV). The invention contemplates that the amount of the pre-selected dose of the agent accumulated in the lymph node is increased by at least 110%, at least 150%, at least 200%, at least 200%, at least 250%, preferably by at least 350% or 3.5×, up to 1750%, the amount accumulated when the agent is administered by routes outside of the intradermal compartment, e.g., SC or IV compartment.

In some embodiments, the concentration of the biologically active agent accumulated in the lymph node after ID delivery is about 5 nanograms of the agent per 50 micrograms of the tissue; 10 picograms of the agent per 50 micrograms of the tissue; 29 nanograms of the agent per 50 micrograms of the tissue; 10 picograms of the agent per 50 micrograms of the tissue to about 29 nanograms of the agent per 50 micrograms of the tissue; 10 picograms of the agent per 50 micrograms of the tissue to about 150 nanograms of the agent per 50 micrograms of the tissue.

In other embodiments, the concentration of the biologically active agent, e.g., a therapeutic or diagnostic agent, accumulated in the lymph node after ID delivery is about 10 pg to about 15 ug of the agent per 50 micrograms of the tissue, or about 1 ng to about 30 ng of the agent per 50 micrograms of the tissue.

In some embodiments, the invention encompasses targeted intradermal delivery of a biologically active agent to any biological entity including but not limited to a cell, a group or collection of cells, a bacteria (e.g., Escherichia coli, Klebsiella pneumoniae, Staphylococcus aureus, Enterococcus faecials, Candida albicans, Proteus vulgaris, Staphylococcus viridans, and Pseudomonas aeruginosa), a pathogen (e.g., B-lymphotropic papovavirus (LPV); Bordatella pertussis; Boma Disease virus (BDV); Bovine coronavirus; Choriomeningitis virus; Dengue virus; a virus, E. coli; Ebola; Echovirus 1; Echovirus-11 (EV); Endotoxin (LPS); Enteric bacteria; Enteric Orphan virus; Enteroviruses; Feline leukemia virus; Foot and mouth disease virus; Gram-negative bacteria; Heliobacter pylori; Hepatitis B virus (HBV); Herpes Simplex Virus; HIV-1; Human cytomegalovirus; Human coronovirus; Influenza A, B & C ; Legionella; Leishmania mexicana; Listeria monocytogenes; Measles virus; Meningococcus; Morbilliviruses; Mycobacterium avium-M; Neisseria gonorrhoeae; Newcastle disease virus; Parvovirus B19; Plasmodium falciparum; Pox Virus; Pseudomonas; Rotavirus; Samonella typhiurium; Shigella; Streptococci; T-cell lymphotropic virus 1; Vaccinia virus); a plaque; a parasitic agent; fungi and yeast. Once an agent is delivered to a biological entity in accordance with the methods of the invention, any of the detection, imaging methods known to one skilled in the art and disclosed herein can be used to detect and image the entity. The methods of the invention encompass methods for delivering a biologically active agent where the agent specifically binds a biological entity.

Biologically active agents, including diagnostic and therapeutic agents delivered in accordance with the methods of the invention preferably will achieve higher maximum concentrations of the agents. The inventors have found that agents administered to the ID compartment are absorbed more rapidly, with bolus administration resulting in higher initial concentrations. Therefore, the dose may be reduced, providing an economic benefit, as well as a physiological benefit since lesser amounts of the drug or diagnostic agent has to be cleared by the body.

In accordance with the invention, direct intradermal (ID) administration can be achieved using, for example, microneedle-based injection and infusion systems or any other means known to one skilled in the art to accurately target the intradermal compartment. Exemplary devices include those disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003; and U.S. Publication No. 2005-0163711 A1, published Jun. 28, 2005; and all of which are incorporated herein by reference in their entirety. Micro-cannula- and microneedle-based methodology and devices are also described in U.S. Publication No. 2002-0095134, published Jul. 18, 2002, which is incorporated herein by reference in its entirety. Standard steel cannula can also be used for intra-dermal delivery using devices and methods as described in U.S. Pat. No. 6,494,865, issued Dec. 17, 2002, which is incorporated herein by reference in its entirety. These methods and devices include the delivery of agents through narrow gauge (30 G or narrower) “micro-cannula” with a limited depth of penetration (typically ranging from 10 μm to 2 mm), as defined by the total length of the cannula or the total length of the cannula that is exposed beyond a depth-limiting hub feature.

The subject of intradermal delivery of the present invention is a mammal, preferably, a human. The biologically active agents delivered in accordance with the methods of the invention (with or without a tracer reagent) may be delivered into the intradermal compartment by a needle or cannula, usually from about 300 μm to about 5 mm long. Preferably, the needle or cannula is about 300 μm to about 1 mm long, with the outlet inserted into the skin of the subject to a depth of 0.5 mm to 1.5 mm. Preferably, a small gauge needle or cannula, between 30 and 36 gauge, preferably 31-34 gauge is used. The outlet of the needle or cannula is preferably inserted to a depth of 0.3 mm (300 μm) to 1 mm.

In one embodiment, the intradermal delivery of active agents in accordance with the present invention are particularly beneficial in the treatment or diagnosis of the diseases including, but not limited to, chronic and acute diseases which include, but are not limited to, lymphoma, melanoma, leukemia, breast cancer, colorectal cancer, cancer metastasis, diseases of the lymphatic system, any disease affecting the lymph nodes, e.g., axillary, politeal, lingual, viral diseases, e.g., HIV. In the case of diagnosis, although not intending to be bound by a particular mechanism of action, diagnostic agents delivered in accordance with the methods of the invention are taken up by the intradermal compartment and delivered to the lymphatic system where its recognition and binding indicate the presence of a cell or disease state. The present invention is also useful for diagnostic procedures including, but not limited to, surgical methods, biopsies, non-invasive screening and image-guided biopsies, image-guided surgery.

The methods of the invention may be particularly useful for methods of integrated diagnosis and therapy, including complementary and/or concurrent diagnostics and monitoring. Accurate diagnosis of a disease is largely an unmet need for example in oncology, where few diagnostic agents indicate which therapeutic choices will succeed with any reliability. The methods of the invention provide delivering agents which specifically recognize a cell, e.g., a cancer cell, in a particular tissue. Such agents include without limitation antibodies, preferably therapeutic monoclonal antibodies disclosed herein. In a specific embodiment, the invention encompasses delivering Herceptin, a monoclonal antibody specific for Her2/neu positive breast cancer to the ID compartment of a subject's skin for improved diagnosis and therapy. The methods of the invention provide improved diagnosis of cancer subjects over traditional methods of diagnostic of Her2/neu positive cancer cells, which identifies the population that will most benefit from this therapeutic treatment while eliminating others that would not. Currently, such in vitro diagnostic tests identifying the population that will most benefit from a particular therapeutic treatment produce “equivocal” or unclear results. By using the methods, devices and particulate formulations of the present invention, identification of the Her2/neu positive cells can be enhanced, for example with a particle labeled with a Her2/neu specific antibody and an encapsulated dye that can be tracked ex vivo. Thus, in vivo intradermal administration of Herceptin labeled particle or a particle that releases a complementary nucleic acid with a signal generator that identifies the mRNA coding for Her2/neu, provides for the ability to identify those individuals suitable for integrated diagnostics and monitoring. In some embodiments, using the methods of this invention, the cells are left intact providing a greater chance for positive identification and confirmation.

The methods of the instant invention provide improved methods for tailoring therapies for a disease, disorder or infection using integrating diagnostic methods of the invention. The methods of the invention are applicable for current tailored and non-tailored treatment regimens. The methods of the invention allow a continuous monitoring of a treatment regimen in a subject. While tailored therapies of the future may require integrated diagnostics, current non-tailored treatment regimens could also benefit from tailored diagnostics of the instant invention. For example, those subjects diagnosed with large diffuse B cell lymphoma typically undergo CHOP therapy. Monitoring the effectiveness of this combined drug regimen is restricted to clinical changes and intermittent non-specific imaging and tissue biopsies. The ability to continually monitor treatment effectiveness would allow for earlier identification of drug resistance and metastasis. This could be accomplished with the administration of specific intradermal diagnostic reagents in the therapeutic cocktail or in combination with existing therapies.

In some embodiments, the methods of the invention encompasses administration of formulations comprising one or more diagnostic agents in combination with one or more therapeutic agents. The present invention provides methods to target diagnostic agents and therapeutic agents to cells of interest. In a specific embodiment, the invention encompasses delivering a diagnostic agent combined with a therapeutic agent to the ID compartment of a subject's skin such that a specific action of the diagnostic agent triggers an action of the therapeutic agent. The combination of targeted diagnostic delivery with targeted therapeutics delivery in accordance with the methods of the invention provides for enhanced patient care. This embodiment teaches the advantages of combining intradermal therapeutic delivery with diagnostic agents. The combination of delivering a diagnostic and a therapeutic agent to the ID compartment, may provide a powerful tool for improving the treatment of a disease in a subject.

In yet other embodiments, the invention enables the use of specific agents, e.g., diagnostic agents, for binding and/or detecting a cellular event or disease state in vivo. As a result, the invention provides screening methods to identify a specific agent needed to bind to the cell of interest. In some embodiments, the invention provides methods for in vivo screening of combinatorial libraries, both biological and chemical, to identify suitable agents (e.g., diagnostic target or moiety or therapeutic target or moiety) in the library for the purpose being tested. The ability to screen for agents in vivo using the methods of the instant invention enables identification of unique cellular and disease states.

In a specific embodiment, the invention provides using an animal model of interest, where libraries of agents can be injected intradermally and their effects monitored over time. Effects which can be monitored include for example relief of symptoms or binding to a tissue and/or cell of interest. In a preferred specific embodiment, an animal tumor model, e.g., a lymphoma mouse model could be used for screening biologically active agents, delivered intradermally that traffic to the lymph nodes. This would enable the detection of cancer cell states in vivo and possibly identify the active triggers for metastases and potential targets for therapeutic and diagnostic agents. These results would then be utilized to develop novel diagnostics for humans and other species.

5.1 Compositions of the Invention

The invention encompasses compositions comprising one or more biologically active agents in particles that preferably can remain suspended in solution for hours, days, weeks, months and/or years. The particles may also exist as emulsions or progress through an emulsion phase during manufacturing. Compositions for use in the methods of the invention may be obtained from any species or generated by any recombinant DNA technology known to one skilled in the art. Compositions comprising one or more biologically active agents may be from different animal species including, but not limited to, swine, bovine, ovine, equine, chicken, murine, rat, human, etc. The chemical state of such agents may be modified by standard recombinant DNA technology to produce agents of different chemical formulas in different association states.

The biologically active agent used in the methods of the invention encompasses any molecule that either specifically or non-specifically binds a molecule in vivo and is capable of producing a biological effect in vivo. The biologically active agents may either be naturally occurring molecules or those derived using a synthetic process or recombinant process, using common methods known to one skilled in the art. Biologically active agents of the invention may recognize specifically or non-specifically a recognition moiety on a particular cell in a particular tissue. Often, these specific agents contain structural or functional properties in common with known biological entities. These biologically active agents may either be naturally occurring recognition molecules or those derived using a synthetic process or recombinant process, using common methods known to one skilled in the art.

In other embodiments, the biologically active agent is a biomimetic in nature, comprising naturally occurring structural motifs while incorporating additional or modified finctional groups for transport, targeting, enhanced binding, stability, or detection.

Examples of biologically active agents that can be used in the methods of the instant invention include without limitation, immunoglobulins (e.g., Multi-specific Igs, Single chain Igs, Ig fragments), proteins, peptides (e.g., peptide receptors, selectins, binding proteins (maltose binding protein, glucose binding protein)), nucleotides, nucleic acids (e.g. RNAs, modified RNA/DNA, aptamers), receptors (e.g., Acetylcholine receptor), enzymes, carbohydrates, glycoproteins (e.g, NCAMs, Sialic acids), bacteriophage (e.g., filamentous phage), viruses (e.g., HIV), chemospecific agents, chemotoxic agents, cytokines and apoptosis inhibitors.

Particularly preferred biologically active agents that may be used in the instant invention are therapeutic antibodies that can be used diagnostically which include but are not limited to HERCEPTIN® (Trastuzumab) (Genentech, CA) which is a humanized anti-HER2 monoclonal antibody for the treatment of patients with metastatic breast cancer; REOPRO® (abciximab) (Centocor) which is an anti-glycoprotein IIIb/IIIa receptor on the platelets for the prevention of clot formation; ZENAPAX® (daclizumab) (Roche Pharmaceuticals, Switzerland) which is an immunosuppressive, humanized anti-CD25 monoclonal antibody for the prevention of acute renal allograft rejection; PANOREX™ which is a murine anti-7-IA cell surface antigen IgG2a antibody (Glaxo Wellcome/Centocor); BEC2 which is a murine anti-idiotype (GD3 epitope) IgG antibody (ImClone System); IMC-C225 which is a chimeric anti-EGFR IgG antibody (ImClone System); VITAXIN™ which is a humanized anti-αVβ3 integrin antibody (Applied Molecular Evolution/MedImmune); Campath 1H/LDP-03 which is a humanized anti CD52 IgG1 antibody (Leukosite); Smart M195 which is a humanized anti-CD33 IgG antibody (Protein Design Lab/Kanebo); RITUXAN™ which is a chimeric anti-CD20 IgGI antibody (IDEC Pharm/Genentech, Roche/Zettyaku); LYMPHOCIDE™ which is a humanized anti-CD22 IgG antibody (Immunomedics); ICM3 is a humanized anti-ICAM3 antibody (ICOS Pharm); IDEC-114 is a primatied anti-CD80 antibody (IDEC Pharn/Mitsubishi); ZEVALIN™ is a radiolabelled murine anti-CD20 antibody (IDEC/Schering AG); IDEC- 131 is a humanized anti-CD40L antibody (IDEC/Eisai); IDEC-151 is a primatized anti-CD4 antibody (IDEC); IDEC-152 is a primatized anti-CD23 antibody (IDEC/Seikagaku); SMART anti-CD3 is a humanized anti-CD3 IgG (Protein Design Lab); 5G1.1 is a humanized anti-complement factor 5 (C5) antibody (Alexion Pharm); D2E7 is a humanized anti-TNF-α antibody (CAT/BASF); CDP870 is a humanized anti-TFN-α Fab fragment (Celltech); IDEC-151 is a primatized anti-CD4 IgG1 antibody (IDEC Pharm/SmithKline Beecham); MDX-CD4 is a human anti-CD4 IgG antibody (Medarex/Eisai/Genmab); CDP571 is a humanized anti-TNF-α IgG4 antibody (Celltech); LDP-02 is a humanized anti-α4 β7 antibody (LeukoSite/Genentech); OrthoClone OKT4A is a humanized anti-CD4 IgG antibody (Ortho Biotech); ANTOVA™ is a humanized anti-CD40L IgG antibody (Biogen); ANTEGREN™ is a humanized anti-VLA-4 IgG antibody (Elan); and CAT-152 is a human anti-TGF-β₂ antibody (Cambridge Ab Tech). These antibodies are preferably encapsulated or bound to the surface of the particles described herein.

Other examples of antibodies, preferably attached to the surface of the particle or encapsulated, that can be used in accordance with the instant invention are listed in Table 1 below.

TABLE 1
Monoclonal antibodies for cancer therapy that can be used in accordance with
the invention.

| Company | Product | Disease | Target |
|---|---|---|---|
| Abgenix | ABX-EGF | Cancer | EGF receptor |
| AltaRex | OvaRex | ovarian cancer | tumor antigen |
| | | | CA125 |
| | BravaRex | metastatic cancers | tumor antigen |
| | | | MUC1 |
| Antisoma | Theragyn | ovarian cancer | PEM antigen |
| | (pemtumomabytrrium- | | |
| | 90) | | |
| | Therex | breast cancer | PEM antigen |
| Boehringer | blvatuzumab | head & neck cancer | CD44 |
| Ingelheim | | | |
| Centocor/J&J | Panorex | Colorectal cancer | 17-1A |
| Corixa | Bexocar | NHL | CD20 |
| CRC | MAb, idiotypic 105AD7 | colorectal cancer | gp72 |
| Technology | | vaccine | |
| Crucell | Anti-EpCAM | cancer | Ep-CAM |
| Cytoclonal | MAb, lung cancer | non-small cell lung | NA |
| | | cancer | |
| Genentech | Herceptin | metastatic breast | HER-2 |
| | | cancer | |
| | Herceptin | early stage breast | HER-2 |
| | | cancer | |
| | Rituxan | Relapsed/refractory | CD20 |
| | | low-grade or | |
| | | follicular NHL | |
| | Rituxan | intermediate & high- | CD20 |
| | | grade NHL | |
| | MAb-VEGF | NSCLC, metastatic | VEGF |
| | MAb-VEGF | Colorectal cancer, | VEGF |
| | | metastatic | |
| IDEC | Zevalin (Rituxan + yttrium- | low grade of | CD20 |
| | 90) | follicular, relapsed | |
| | | or refractory, CD20- | |
| | | positive, B-cell NHL | |
| | | and Rituximab- | |
| | | refractory NHL | |
| ImClone | Cetuximab + innotecan | refractory colorectal | EGF receptor |
| | | carcinoma | |
| | Cetuximab + cisplatin & | newly diagnosed or | EGF receptor |
| | radiation | recurrent head & | |
| | | neck cancer | |
| | Cetuximab + gemcitabine | newly diagnosed | EGF receptor |
| | | metastatic pancreatic | |
| | | carcinoma | |
| | Cetuximab + cisplatin + 5FU | recurrent or | EGF receptor |
| | or Taxol | metastatic head & | |
| | | neck cancer | |
| | Cetuximab + carboplatin + paclitaxel | newly diagnosed | EGF receptor |
| | | non-small cell lung | |
| | | carcinoma | |
| | Cetuximab + cisplatin | head & neck cancer | EGF receptor |
| | | (extensive incurable | |
| | | local-regional | |
| | | disease & distant | |
| | | metasteses) | |
| | Cetuximab + radiation | locally advanced | EGF receptor |
| | | head & neck | |
| | | carcinoma | |
| | BEC2 + Bacillus | small cell lung | mimics |
| | Calmette Guerin | carcinoma | ganglioside GD3 |
| | BEC2 + Bacillus | melanoma | mimics |
| | Calmette Guerin | | ganglioside GD3 |
| | IMC-1C11 | colorectal cancer | VEGF-receptor |
| | | with liver metasteses | |
| ImmonoGen | nuC242-DM1 | Colorectal, gastric, | nuC242 |
| | | and pancreatic | |
| | | cancer | |
| ImmunoMedics | LymphoCide | Non-Hodgkins | CD22 |
| | | lymphoma | |
| | LymphoCide Y-90 | Non-Hodgkins | CD22 |
| | | lymphoma | |
| | CEA-Cide | metastatic solid | CEA |
| | | tumors | |
| | CEA-Cide Y-90 | metastatic solid | CEA |
| | | tumors | |
| | CEA-Scan (Tc-99m- | colorectal cancer | CEA |
| | labeled arcitumomab) | (radioimaging) | |
| | CEA-Scan (Tc-99m- | Breast cancer | CEA |
| | labeled arcitumomab) | (radioimaging) | |
| | CEA-Scan (Tc-99m- | lung cancer | CEA |
| | labeled arcitumomab) | (radioimaging) | |
| | CEA-Scan (Tc-99m- | intraoperative | CEA |
| | labeled arcitumomab) | tumors (radio | |
| | | imaging) | |
| | LeukoScan (Tc-99m- | soft tissue infection | CEA |
| | labeled sulesomab) | (radioimaging) | |
| | LymphoScan (Tc-99m- | lymphomas | CD22 |
| | labeled) | (radioimaging) | |
| | AFP-Scan (Tc-99m- | liver 7 gem-cell | AFP |
| | labeled) | cancers | |
| | | (radioimaging) | |
| Intracel | HumaRAD-HN (+yttrium- | head & neck cancer | NA |
| | 90) | | |
| | HumaSPECT | colorectal imaging | NA |
| Medarex | MDX-101 (CTLA-4) | Prostate and other | CTLA-4 |
| | | cancers | |
| | MDX-210 (her-2 | Prostate cancer | HER-2 |
| | overexpression) | | |
| | MDX-210/MAK | Cancer | HER-2 |
| MedImmune | Vitaxin | Cancer | αvβ₃ |
| Merck KGaA | MAb 425 | Various cancers | EGF receptor |
| | IS-IL-2 | Various cancers | Ep-CAM |
| Millennium | Campath (alemtuzumab) | chronic lymphocytic | CD52 |
| | | leukemia | |
| NeoRx | CD20-streptavidin (+biotin- | Non-Hodgkins | CD20 |
| | yttrium 90) | lymphoma | |
| | Avidicin (albumin + NRLU13) | metastatic cancer | NA |
| Peregrine | Oncolym (+iodine-131) | Non-Hodgkins | HLA-DR 10 beta |
| | | lymphoma | |
| | Cotara (+iodine-131) | unresectable | DNA-associated |
| | | malignant glioma | proteins |
| Pharmacia | C215 (+staphylococcal | pancreatic cancer | NA |
| Corporation | enterotoxin) | | |
| | MAb, lung/kidney cancer | lung & kidney | NA |
| | | cancer | |
| | nacolomab tafenatox | colon & pancreatic | NA |
| | (C242 + staphylococcal | cancer | |
| | enterotoxin) | | |
| Protein Design | Nuvion | T cell malignancies | CD3 |
| Labs | SMART M195 | AML | CD33 |
| | SMART 1D10 | NHL | HLA-DR antigen |
| Titan | CEAVac | colorectal cancer, | CEA |
| | | advanced | |
| | TriGem | metastatic melanoma | GD2-ganglioside |
| | | & small cell lung | |
| | | cancer | |
| | TriAb | metastatic breast | MUC-1 |
| | | cancer | |
| Trilex | CEAVac | colorectal cancer, | CEA |
| | | advanced | |
| | TriGem | metastatic melanoma | GD2-ganglioside |
| | | & small cell lung | |
| | | cancer | |
| | TriAb | metastatic breast | MUC-1 |
| | | cancer | |
| Viventia | NovoMAb-G2 | Non-Hodgkins | NA |
| Biotech | radiolabeled | lymphoma | |
| | Monopharm C | colorectal & | SK-1 antigen |
| | | pancreatic carcinoma | |
| | GlioMAb-H (+gelonin | gliorna, melanoma & | NA |
| | toxin) | neuroblastoma | |
| Xoma | Rituxan | Relapsed/refractory | CD20 |
| | | low-grade or | |
| | | follicular NHL | |
| | Rituxan | intermediate & high- | CD20 |
| | | grade NHL | |
| | ING-1 | adenomcarcinoma | Ep-CAM |

In one specific embodiment, the invention encompasses particulate compositions comprising biologically active agents comprising one or more diagnostic agents. In another specific embodiment, the invention encompasses particulate compositions comprising biologically active agents which comprise at least one diagnostic and at least one therapeutic agent. In one embodiment, the biologically active particulate agent comprises a ligand that identifies the cell type of a particular disease or disorder (e.g., a cancer), along with a therapeutic agent, e.g., an agent capable of killing diseased cells. For example, a ligand to a marker identifying an undesirable cell type may be conjugated with a toxin capable of inactivating or killing the target cells and be carried to the location of the target cell by the particles described herein.

Therapeutic agents that may be used in the compositions of the invention include but are not limited to chemotherapeutic agents, radiation therapeutic agents, hormonal therapeutic agents, immunotherapeutic agents, immunomodulatory agents, anti-inflammatory agents, antibiotics, anti-viral agents, and cytotoxic agents.

The particles described herein may also be used to transport anti-inflamatory agents. Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

Examples of immunomodulatory agents include, but are not limited to, methothrexate, ENBREL, REMICADE™, leflunomide, cyclophosphamide, cyclosporine A, and macrolide antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamindes (e.g., leflunamide), T cell receptor modulators, and cytokine receptor modulators, corticosteroids, cytokine agonists, cytokine antagonists, and cytokine inhibitors.

Examples of antibiotics include, but are not limited to, macrolide (e.g., tobramycin (Tobi®)), a cephalosporin (e.g., cephalexin (Keflex®), cephradine (Velosef®), cefuroxime (Ceftin®), cefprozil (Cefzil®), cefaclor (Ceclor®), cefixime (Suprax®) or cefadroxil (Duricef®)), a clarithromycin (e.g., clarithromycin (Biaxin®)), an erythromycin (e.g., erythromycin (EMycin®)), a penicillin (e.g., penicillin V (V-Cillin K® or Pen Vee K®)) or a quinolone (e.g., ofloxacin (Floxin®), ciprofloxacin (Cipro®) or norfloxacin (Noroxin®)),aminoglycoside antibiotics (e.g., apramycin, arbekacin, bambermycins, butirosin, dibekacin, neomycin, neomycin, undecylenate, netilmicin, paromomycin, ribostamycin, sisomicin, and spectinomycin), amphenicol antibiotics (e.g., azidamfenicol, chloramphenicol, florfenicol, and thiamphenicol), ansamycin antibiotics (e.g., rifamide and rifampin), carbacephems (e.g., loracarbef), carbapenems (e.g., biapenem and imipenem), cephalosporins (e.g., cefaclor, cefadroxil, cefamandole, cefatrizine, cefazedone, cefozopran, cefpimizole, cefpiramide, and cefpirome), cephamycins (e.g., cefbuperazone, cefmetazole, and cefminox), monobactams (e.g., aztreonam, carumonam, and tigemonam), oxacephems (e.g., flomoxef, and moxalactam), penicillins (e.g., amdinocillin, amdinocillin pivoxil, amoxicillin, bacampicillin, benzylpenicillinic acid, benzylpenicillin sodium, epicillin, fenbenicillin, floxacillin, penamccillin, penethamate hydriodide, penicillin o-benethamine, penicillin 0, penicillin V, penicillin V benzathine, penicillin V hydrabamine, penimepicycline, and phencihicillin potassium), lincosamides (e.g., clindamycin, and lincomycin), amphomycin, bacitracin, capreomycin, colistin, enduracidin, enviomycin, tetracyclines (e.g., apicycline, chlortetracycline, clomocycline, and demeclocycline), 2,4-diaminopyrimidines (e.g., brodimoprim), nitrofurans (e.g., furaltadone, and furazolium chloride), quinolones and analogs thereof (e.g., cinoxacin, clinafloxacin, flumequine, and grepagloxacin), sulfonamides (e.g., acetyl sulfamethoxypyrazine, benzylsulfamide, noprylsulfamide, phthalylsulfacetamide, sulfachrysoidine, and sulfacytine), sulfones (e.g., diathymosulfone, glucosulfone sodium, and solasulfone), cycloserine, mupirocin, chloramphenicols, erythromycin, penicillin, streptomycin, vancomycin, trimethoprimsulfamethoxazols, and tuberin.

Examples of anti-viral agents include, but are not limited to, protease inhibitors, nucleoside reverse transcriptase inhibitors, non-nucleoside reverse transcriptase inhibitors and nucleoside analogs, zidovudine, acyclovir, gangcyclovir, vidarabine, idoxuridine, trifluridine, and ribavirin, as well as foscarnet, amantadine, rimantadine, saquinavir, indinavir, amprenavir, lopinavir, ritonavir, the alpha-interferons; adefovir, clevadine, entecavir, and pleconaril

Other therapeutic agents which can be used with the present invention include but are not limited to Alpha-1 anti-trypsin, Anti-Angiogenesis agents, Antisense, butorphanol, Calcitonin and analogs, Ceredase, COX-II inhibitors, dermatological agents, dihydroergotamine, Dopamine agonists and antagonists, Enkephalins and other opioid peptides, Epidermal growth factors, Erythropoietin and analogs, Follicle stimulating hormone, G-CSF, Glucagon, GM-CSF, granisetron, Growth hormone and analogs (including growth hormone releasing hormone), Growth hormone antagonists, Hirudin and Hirudin analogs such as Hirulog, IgE suppressors, Insulin, insulinotropin and analogs, Insulin-like growth factors, Interferons, Interleukins, Luteinizing hormone, Luteinizing hormone releasing hormone and analogs, Heparins, Low molecular weight heparins and other natural, modified, or synthetic glycoaminoglycans, M-CSF, metoclopramide, Midazolam, Monoclonal antibodies, Pegylated antibodies, Pegylated proteins or any proteins modified with hydrophilic or hydrophobic polymers or additional finctional groups, Fusion proteins, Single chain antibody fragments or the same with any combination of attached proteins, macromolecules, or additional finctional groups thereof, Narcotic analgesics, nicotine, Non-steroid anti-inflammatory agents, Oligosaccharides, ondansetron, Parathyroid hormone and analogs, Parathyroid hormone antagonists, Prostaglandin antagonists, Prostaglandins, Recombinant soluble receptors, scopolamine, Serotonin agonists and antagonists, Sildenafil, Terbutaline, and other therapeutics such as agents for the common cold, Anti-addiction, anti-allergy, anti-emetics, anti-obesity, antiosteoporeteic, anti-infectives, analgesics, anesthetics, anorexics, antiarthritics, antiasthmatic agents, anticonvulsants, antidepressants, antidiabetic agents, antihistamines, anti-inflammatory agents, antimigraine preparations, antimotion sickness preparations, antinauseants, antineoplastics including vindesine, antiparkinsonism drugs, antipruritics, antipsychotics, antipyretics, anticholinergics, benzodiazepine antagonists, vasodilators, including general, coronary, peripheral and cerebral, bone stimulating agents, central nervous system stimulants, hormones, hypnotics, immunosuppressives, muscle relaxants, parasympatholytics, parasympathomimetrics, prostaglandins, proteins, peptides, polypeptides and other macromolecules, psychostimulants, sedatives, and sexual hypofunction and tranquilizers.

Conjugates may also be transported using the particles described herein. A biologically active agent, e.g., a diagnostic or therapeutic agent, may be conjugated to a therapeutic moiety such as a cytotoxin (e.g., a cytostatic or cytocidal agent), a therapeutic agent or a radioactive element (e.g., alpha-emitters, gamma-emitters, etc.). Cytotoxins or cytotoxic agents include any agent that is detrimental to cells. Examples include paclitaxel, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Therapeutic agents include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine), prednisone and adriomycin.

Moreover, a biologically active agent can be conjugated to therapeutic moieties such as a radioactive materials or macrocyclic chelators useful for conjugating radiometal ions (see above for examples of radioactive materials). In certain embodiments, the macrocyclic chelator is 1,4,7,10-tetraazacyclododecane-N,N′,N″,N″-tetraacetic acid (DOTA) which can be attached to the antibody via a linker molecule. Such linker molecules are commonly known in the art and described in Denardo et al., 1998, Clin Cancer Res. 4:2483-90; Peterson et al., 1999, Bioconjug. Chem. 10:553; and Zimmerman et al., 1999, Nucl. Med. Biol. 26:943-50 each incorporated by reference in their entireties.

Techniques for conjugating such therapeutic moieties to biologically active agents, e.g., antibodies, are well known; see, e.g., Arnon et al., “Monoclonal Antibodies For Immunotargeting Of Drugs In Cancer Therapy”, in Monoclonal Antibodies And Cancer Therapy, Reisfeld et al. (eds.), 1985, pp. 243-56, Alan R. Liss, Inc.); Hellstrom et al., “Antibodies For Drug Delivery”, in Controlled Drug Delivery (2nd Ed.), Robinson et al. (eds.), 1987, pp. 623-53, Marcel Dekker, Inc. ); Thorpe, “Antibody Carriers Of Cytotoxic Agents In Cancer Therapy: A Review”, in Monoclonal Antibodies '84: Biological And Clinical Applications, Pinchera et al. (eds.), 1985, pp. 475-506); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), 1985, pp. 303-16, Academic Press; and Thorpe et al., Immunol. Rev., 62:119-58, 1982.

In some embodiments, the particulate compositions of the invention comprise an effective amount of a biologically active agent and one or more other additives, beyond the molecules that form the particle structure. Additives that may be used in the compositions of the invention include for example, wetting agents, emulsifying agents, or pH buffering agents. The compositions of the invention may contain one or more other excipients such as saccharides and polyols. Additional examples of pharmaceutically acceptable carriers, diluents, and other excipients are provided in Remington's Pharmaceutical Sciences (Mack Pub. Co. N.J. current edition, all of which is incorporated herein by reference in its entirety.

The invention encompasses compositions in which the biologically active agent is in a particulate form, i.e., is not fully dissolved in solution. In some embodiments, at least 30%, at least 50%, at least 75% of the biologically active agent is in particulate form. Although not intending to be bound by a particular mode of action, compositions of the invention in which a biologically active agent is in particulate form have at least one agent which facilitates the precipitation of the agent. Precipitating agents that may be employed in the compositions of the invention may be proteinacious, e.g., protamine, a cationic polymer, or non-proteinacious, e.g., zinc or other metals or polymers.

In some embodiments, a tracer agent may be concurrently administered with the biologically active agent described herein to facilitate the tracing and examination of the biologically active agent. The tracer agent may include, but is not limited to, visible dyes, fluorescent dyes, NIR dyes, radioisotopes, or magnetic spin labels. Such tracer agents can be easily observed by the conventional techniques. Detection of the labeled agents or the tracer agents may be accomplished using ex vivo or in vivo, invasive or non-invasive, using methods known in the art.

The biologically active agent (in particulate form) may be delivered or administered in solutions of pharmaceutically acceptable diluents or solvents, emulsions, suspensions, gels, other particulates such as micro- and nanoparticles either suspended or dispersed, as well as in-situ forming vehicles of the same. The compositions of the invention may be in any form suitable for intradermal delivery. In one embodiment, the intradermal composition of the invention is in the form of a flowable, injectable medium, i.e., a low viscosity composition that may be injected in a syringe or pen. The flowable injectable medium may be a liquid. Alternatively the flowable injectable medium is a liquid in which particulate material is suspended, such that the medium retains its fluidity to be injectable and syringable, e.g., can be administered in a syringe.

The invention encompasses formulations comprising at least one biologically active agent, wherein the concentration of the agent is between about 20 ug/mL to 100 mg/mL. In a specific embodiment, the concentration of the agent is about 1 ug/mL, 10 ug/mL, 1 mg/mL, or 10 mg/mL. In another specific embodiment, the concentration of the agent is about 20 mg/mL, 50 mg/mL, 70 mg/mL, or 100 mg/mL. In some embodiments, the amount of the at agent delivered in accordance with the methods of the invention is between about 1 to 100, I to 10, 0.1 to 10, or 5 to 10 ug.

The invention also includes compositions comprising particle reagents for diagnostic and/or therapeutic use and methods of delivery thereof. In brief, particles of defined shape and surface characteristics may be suspended in liquid media and delivered for example through micro needles to the intradermal compartment, e.g., generally less than 5 mm below the epidermis and preferably between 1 and 3 mm below the epidermis. These particles are then transported through the lymphatic vasculature to lymph nodes. Particle migration rate may be contingent on size and surface charge.

As used herein, the term “particles” includes any formed element such as a lattice or sac comprising monomers, polymers, lipids, amphiphiles, fatty acids, steroids, proteins, and other materials, many of which are known to aggregate or self-assemble, or which can be processed into particles. Particles also include unilamelar, multilamelar, random tortuous path and solid morphologies including but not limited to liposomes, microcrystalline materials, particulate MRI contrast agents, polymeric beads (i.e., latex and HEMA), but most preferably hollow particles, such as microcapsules or microbubbles, which are particularly useful for ultrasonic imaging and capable of carrying payload to the specifications described herein.

In one embodiment, the invention encompasses particles comprising one or more biologically active agents including therapeutic and diagnostic agents which may result in site selective non-invasive dissolution of said particles to deliver the agent. In a specific embodiment, the invention encompasses compositions comprising a detectable agent (e.g., creating an intradermal/lymphatic echogenic microbubble) and comprise a therapeutic agent, e.g., doxorubicin. Although not intending to be bound by a particular mechanism of action, once introduced the particles are actively or passively trafficked into the area and regional draining lymph nodes. As the particles move into these tissues an ultrasound probe detects their presence and, at the appropriate frequency, breaks the particle open; its contents then diffuse into nearby tissues allowing for high local agent concentration only at the disease locus without need for systemic delivery. Additionally, the particle may further comprise a diagnostic agent so that dispersion of the agent is limited to the immediate tissue for additional analysis.

The potential advantages of such particle delivery systems include, but are not limited to: (1) improved targeting of the lymphatic system tissue via targeted ID delivery (using such delivery systems, disease response can occur in the lymphatic tissue and direct access to this process may offer greater effectiveness of therapeutics and improved diagnostic capabilities); and (2) improved therapeutic and diagnostic outcomes. Local delivery of therapeutics to tissue of greatest interest offers the possibility of improved clinical outcomes due to altered PK and PD profiles. With local delivery in accordance with the methods of the invention, less agent than traditional systemic delivery may be used in order to achieve the desired clinical or diagnostic outcome with the associated decrease in side effects. The methods of the invention result in an increased sensitivity and speed for diagnostic assessment due to local delivery of high concentration agent.

Particles as described herein are delivered intradermally and may be, non-specific non-tissue binding, or specific tissue and/or cell binding (that is, the particle may bind to a particular biological entity or may have a targeting molecule attached to it), and may be associated with therapeutic or diagnostic moieties via various methods. The particles themselves may be the therapeutic or diagnostic agent or they may encapsulate, entrap, or bind the therapeutic or diagnostic agent. The invention encompasses all drug classes and diagnostic agents. The therapeutic or diagnostic agents used in the methods and compositions of the invention may or may not be cell or tissue targeted.

In some specific embodiments, the particles comprise one or more diagnostic agents. Although not intending to be bound by a particular particles provide signal amplification needed for diagnosis of rare events using imaging methods known in the art and disclosed herein.

In some embodiments, particle reagents may further comprise therapeutic agents which are carried with the particles into the lymphatic system and delivered at rates determined by particle composition. In some specific embodiments, the particles comprise therapeutic agents in combination with one or more diagnostic agents. Although not intending to be bound by a particular mechanism of action, particles provide for extended targeted release of agents to particular tissues and/or organs rather than release to the general circulation. Consequently, toxicity is reduced and therapeutic effect is maximized.

In particularly preferred embodiments, the compositions used in the methods of the invention comprise of nanoparticles.

One preferred embodiment of this aspect of the invention relates to a composition comprising small non-specific microbubbles and a method for delivering the composition using intradermal methods to a particular tissue, e.g., lymphatic tissue, or a particular organ. Although not intending to be bound by a particular mechanism of action, microbubbles are rapidly transported through the lymphatic circulation and may be detected using for example ultrasonic imaging. The invention thus provides improved methods for detecting cancer cells and metastases within the lymphatic system for example to sentinel lymph nodes, and/or improved methods for evaluating lymphedema, e.g., a common morbidity associated with extensive axillary lymph node dissection. The methods of the invention are improved over conventional cancer diagnostics such as those disclosed in, e.g., Creager, A. J.; Geisinger, K. R.; Shiver, S. A.; Perier, N. D.; Shen, P.; Shaw, J.; Young, P. R.; Levine, E. A. “Intraoperative Evaluation of Sentinel Lymph Nodes for Metastatic Breast Carcinoma by Imprint Cytology” Mod Pathol 2002, 15(11), 1140-1146.

In other specific embodiments the invention encompasses hypoxia detection via intradermal delivery of oxygen responsive particles.

The intradermal compositions of the present invention can be prepared as unit dosage forms. A unit dosage per vial may contain 0.1 to 0.5 mL of the composition. In some embodiments, a unit dosage form of the intradermal compositions of the invention may contain 50 μL to 100 μL, 50 μL to 20 μL, or 50 μL to 500 μAL of the composition. If necessary, these preparations can be adjusted to a desired concentration by adding a sterile diluent to each vial. Compositions administered in accordance with the methods of the invention are not administered in volumes whereby the intradermal space might become overloaded leading to partitioning to one or more other compartments, such as the SC compartment.

5.1.1 Liposome Based Particles

In some embodiments, this invention encompasses liposome based formulations wherein an active agent is encapsulated in liposomes. Liposomes encapsulating active agents may be prepared by the methods known in the art, as well as those described herein.

In general, liposomes (lipid vesicles) are formed when a thin film or cake is hydrated. Stacks of liquid crystalline bilayers become fluid and swell. The hydrated lipid sheets detach with agitation and close to form large multimellar vesicles. (LMVs or MLVs).

Once formed the next step requires energy to reduce the particle down to a size that is best for the intended application. The sizing process is simultaneously used to encompass agent (or more agent) for research, diagnostic, vaccination or treatment applications.

Particles can be reduced in size by sonication, extrusion or combustion. The liposomes used in this invention are primarily the products of extrusion. The formed liposomes are pushed through polycarbonate membranes ranging from 5 micron to 30 nanometers, preferably 5 micron to 15 nanometers. Both MLV, REV (reverse phase evaporation) and LUV methods have been used in this invention, although methods such as, but not limited to detergent dialysis may be also used. In the MLV procedure, the initial lipid film was hydrated in an aqueous solution containing the agent to be encapsulated and subsequent extrusion was performed in the presence of the same agent. In the LUV procedure, particles were initially formed in an aqueous solution without agent. A low speed spin in a Beckman CS-6R Centrifuge at 2,000 rpms was performed to remove most of the MLVs. The resulting supernatant contained LUVs that were pelleted during a 40,000 rpm spin in a Beckman Ty50 rotor. The supernatant was removed from the high-speed pellet and replaced with aqueous solution containing the agent for encapsulation. Subsequent extrusion was performed in the presence of the agent.

Any suitable lipids or phospholipids may be used in connection with the liposome compositions, and methods, of the present invention. For example, liposomes or lipid emulsions which comprise cholesterol and at least one of DSPA, DSPC, DSPG, DSPE, HSPC, DSPE PEG 2000, DSPE PEG 750, DSPE PEG 350, DPPG, DOPC, sphingomyelin, dihydrosphingomyelin, or Diol-Biotin, and optionally one of alpha tocopherol or triolein, may be used. Cholesterol may represent on a weight per weight basis 10% to 50% of the encapsulating moiety; independently, or in combination, DSPA, DSPC, DSPG, DSPE, HSPC, DSPE-MPEG 2000, DSPE-PEG 350, DPPG, DOPC, sphingomyelin, dihydrosphingomyelin or Diol-Biotin may represent 1% to 80% of the encapsulating moiety on a weight per weight basis; and one of tocopherol or triolein may represent less than or equal to 10% of the encapsulation moiety on a weight per weight basis. Phospholipids with 14, 16, 18, 20 and 22 carbon chains may be substituted for the phospholipids described above.

For certain liposome preps in this invention Sulfa Rhodamine B was encapsulated. The dye is similar in weight to many of the chemo toxic agents used in cancer therapy. The SRB dye is readily visible to the eye and providing the inventors with a means of viewing real-time drainage from outside the body Encapsulated agent was separated from free agent by centrifugation. Chromatography can also be used. Typically three washes were performed to remove unencapsulated material. A phosphorus assay was performed to standardize one batch to the next, making ready for in-vitro and in-vivo studies.

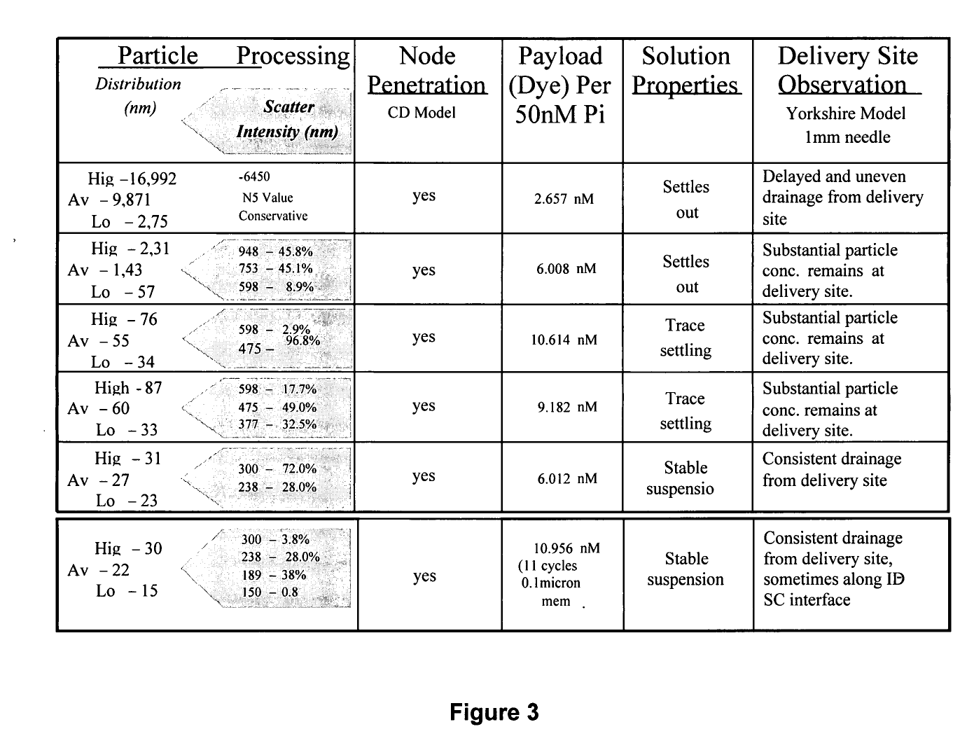

In certain embodiments of the invention, liposomes based particles are referred to using the name given by the inventors. The names and components of such liposome based particles, and their sizes and charges are as shown in Table 2 below, and also in FIG. 3.

TABLE 2
Formulations and Charges of Liposome Particles

| Liposome | | | |
|---|---|---|---|
| Formulation # (F) | Lipid Components and | | Zeta Potential |
| Mean Diameter (nm) | Quantity | | (mV) |
| F4, F5, F9 | Chol | 50.9 mg | −29.29 to −31.37 |
| | DSPG | 10.3 mg | |
| | DSPC | 94.0 mg | |
| F13 | Chol | 50.9 mg | −53.71 |
| | DSPG | 10.3 mg | |
| | DSPC | 86.0 mg | |
| F21, F22, F25, F26, | Chol | 50.9 mg | −27.29 |
| F31 | DSPG | 10.3 mg | |
| | DSPC | 94.0 mg | |
| | Diol-Biotin | 2.6 mg | |
| F35 | Chol | 50.9 mg | −59.49 |
| | DSPG | 57.3 mg | |
| | DSPC | 47.0 mg | |
| F36, F39 | Chol | 50.9 mg | −54.20 |
| | DSPG | 10.3 mg | |
| | DSPC | 71.2 mg | |
| | DSPA | 21.8 mg | |
| F37 | Chol | 50.9 mg | −17.42 |
| | DSPG | 10.3 mg | |
| | DSPC | 94.0 mg | |
| | DSPE-MPEG2000 | 1.47 mg | |
| F50 | Chol | 50.9 mg | |
| | DSPC | 47.0 mg | |
| | DSPG | 57.5 mg | |
| | Diol-Biotin | 2.6 mg | |
| F51 | Chol | 50.9 mg | |
| | DSPG | 10.3 mg | |
| | DSPC | 94.0 mg | |
| | DSPE-PEG350 | 1.47 mg | |
| | Diol-Biotin | 2.6 mg | |
| F58, F60, F72 | Chol | 31.9 mg | −5.77 |
| | HSPC | 95.8 mg | |
| | DSPE-PEG2K | 31.9 mg | |
| F73 | Chol | 34.2 mg | −6.64 |
| | DSPE PEG 2K | 28 mg | |
| | Sphingomyelin | 77.4 mg | |
| F69 | Chol | 30.9 mg | |
| | HSPC | 86.2 mg | |
| | DSPEPEG350 | 30.9 mg | |
| 83 nm | Diol-Biotin | 15.7 mg | |

TABLE 2A
Molar Ratio of Lipid in Formulations and Percent of Membrane
Cholesterol:DSPC:DSPG

| | |
|---|---|
| 10.3:9.2:1 | on a molar ratio |
| 50.2% 44.9% 4.9% | of membrane |
| 2.25:1:1.2 | on a molar ratio |
| 50.5% 22.5% 27% | of membrane |
| alernatively | |
| 50% 1% 49% | of membrane + 0 or up to 10%, preferably 0.1 to |
| | 10% triolein on a wt to wt basis with lipids |
| 50% 49% 1% | of membrane + 0 or up to 10%, preferably 0.1 to |
| | 10% triolein on a wt to wt basis with lipids |
| 30% 1% 69% | of membrane + 0 or up to 1%, preferably 0.1 to |
| | 1% tocopherol on a wt to wt basis with lipids |
| 30% 69% 1% | of membrane + 0 or up to 1%, preferably 0.1 to |
| | 1% tocopherol on a wt to wt basis with lipids |

Cholesterol:HSPC:DSPE PEG 2000

| | |
|---|---|
| 7.6:11.5:1 | on a molar ratio |
| 37.8% 57.3% 4.9% | of membrane |
| alernatively | |
| 50% 45% 5% | of membrane + 0 or up to 10%, preferably 0.1 to |
| | 10% triolein on a wt to wt basis with lipids |
| 50% 49% 1% | of membrane + 0 or up to 10%, preferably 0.1 to |
| | 10% triolein on a wt to wt basis with lipids |
| 30% 65% 5% | of membrane + 0 or up to 1%, preferably 0.1 to |
| | 1% tocopherol on a wt to wt basis with lipids |
| 30% 69% 1% | of membrane + 0 or up to 1%, preferably 0.1 to |
| | 1% tocopherol on a wt to wt basis with lipids |

Cholesterol:Sphingomyelin:DSPE PEG 2000

| | |
|---|---|
| 9:11:1 | on a molar ratio |
| 45% 55% 5% | of membrane |
| alernatively | |
| 50% 45% 5% | of membrane + 0 or up to 10%, preferably 0.1 to |
| | 10% triolein on a wt to wt basis with lipids |
| 50% 49% 1% | of membrane + 0 or up to 10%, preferably 0.1 to |
| | 10% triolein on a wt to wt basis with lipids |
| 30% 65% 5% | of membrane + 0 or up to 1%, preferably 0.1 to |
| | 1% tocopherol on a wt to wt basis with lipids |
| 30% 69% 1% | of membrane + 0 or up to 1%, preferably 0.1 to |
| | 1% tocopherol on a wt to wt basis with lipids |

Cholesterol:HSPC:DSPE PEG 350:Diol-Biotin

| | |
|---|---|
| 84:126:29:1 | on a molar ratio |
| 35% 52.5% 12.1 0.4% | of membrane |
| alernatively | |
| 50% 34% 15.9% 0.1% | of membrane + 0 or up to 10%, |
| | preferably 0.1 to 10% triolein on |
| | a wt to wt basis with lipids |
| 50% 39% 1.0% 10% | of membrane + 0 or up to 10%, |
| | preferably 0.1 to 10% triolein on |
| | a wt to wt basis with lipids |
| 30% 54% 15.9% 0.1% | of membrane + 0 or up to 1%, |
| | preferably 0.1 to 1% tocopherol |
| | on a wt to wt basis with lipids |
| 30% 59% 1.0% 10% | of membrane + 0 or up to 1%, |
| | preferably 0.1 to 1% tocopherol |
| | on a wt to wt basis with lipids |

5.2 Diagnostic and Therapeutic Uses

The present invention provides improved methods for therapy, diagnosis and/or detection of a disease or disorder by improving sensitivity, the amount of the agent deposited, tissue bioavailability, and faster onset of the delivered biologically active agent, e.g., diagnostic or therapeutic agent. The biologically active agents disclosed herein can be used to treat, detect, diagnose, or monitor diseases or disorders. The invention provides a method for administration of at least one therapeutic or diagnostic agent for the treatment or detection of a disease (e.g., cancer) comprising delivering the agent into the ID compartment of a subject's skin at a controlled rate, volume and pressure so that the agent is deposited into the ID compartment and taken up by the lymphatic vasculature.

The methods of the invention also encompass administering a therapeutically or diagnostically effective, preferably non-toxic amount of an agent to a mammal, such that the agent is imageable and detectable with naked eye or with sufficient resolution through the methods disclosed herein and known to one skilled in the art, e.g., ultrasound or magnetic resonance imaging, to permit visualization of intranodal architecture. Preferably, the agents administered in accordance with the methods of the invention are deposited in a particular tissue, e.g., in the lymphatic tissue such as lymph nodes; and the agent is imaged in the subject. The agent may be imaged within about 30 minutes of said administration, within about 4 hours of said administration, within about 24 hours of said administration, or within about 1 month of said administration.

In some embodiments, the invention provides a method for the detection or diagnosis of a disease or disorder, comprising: (a) delivering one or more diagnostic agents to the ID compartment of the subject's skin according to methods of the present invention; (b) assaying the expression of a specific gene product known to have aberrant expression or levels resulting in the disease, disorder, or infection in a subject using one or more agents that specifically bind to a cell expressing the specific gene product; and (c) comparing the level of the expression of the gene with a control level, e.g., levels in normal tissue samples, whereby an increase in the assayed level compared to the control level is indicative of the disease, disorder or infection.

One aspect of the invention is the detection and diagnosis of a disease, disorder, or infection in a human. In one embodiment, diagnosis comprises: (a) administering to a subject an effective amount of a labeled biologically active agent by delivering the agent to the ID compartment of the subject's skin according to methods of the present invention, preferably so that the agent specifically binds a cell that resides in the target tissue; (b) waiting for a time interval following the administration of the agent for permitting the labeled agent to preferentially concentrate at sites, preferably by forming an aggregate or fusion, in the subject where specific binding to the target tissue occurs (and for unbound labeled agent to be cleared to background level); (c) determining background level; and (d) detecting the labeled agent in the subject, such that detection of labeled agent above the background level indicates that the subject has the disease, disorder, or infection. In accordance with this embodiment, the agent may comprise an imaging moiety which is detectable using an imaging system known to one of skill in the art. Background level can be determined by various methods including, comparing the amount of labeled agent detected to a standard value previously determined for a particular system.

In a specific embodiment, the invention encompasses a diagnostic method for cancer comprising the following: antibody specific for a particular cell type, i.e., breast cancer, labeled with a dye that is detectable upon exposure to a specific light source is injected intradermally into and around the tissue of interest. The surgeon using a unique light source (hand held or incorporated into another instrument (e.g., specially designed eyeglasses)) follows the path of the labeled antibody in the lymph nodes looking for metastases and cancer spread. In alternative embodiments, the label is radioactive or magnetic with an appropriate external source to track the label, and in some cases, may be one that is not capable of being detected until the specific agent binds to its target. The diagnostic agents of the invention are particularly useful for cancer prognosis since oxygen concentration proximal to tumors often indicates susceptibility to radiation (see, e.g., Lo et al., 1995, Biochemistry 20, 11,727-11730) and photodynamic therapies (see, e.g., Mcllroy et al., 1998, J Photochem Photobiol, 43, 47-55).

In one embodiment, the present invention provides a method particularly useful for treatment or diagnosis of cancer metastasis. In a preferred diagnostic method, a biologically active agent in particulate form is intradermally delivered to a location suspected of having a tumor, and the biologically active agent is transported to the local lymphatic system so that the lymphatic system, including the lymph nodes draining the location, are identified. Microexamination is then performed on the identified lymph nodes to determine whether cancer cells have migrated into the lymph nodes, i.e., that metastasis has occurred. For example, ProstaScint™ (Cytogen) is an ¹¹In labeled monoclonal antibody used for staging prostate cancer; ⁹⁹TC labeled anti CD-15 monoclonal antibodies have been used for highly sensitive and specific identification of equivocal appendicitis (Kipper, S. L. et. al. Journal ofNuclear Medicine 2000 41(3), 449-455). The invention encompasses administration of Cytogen to the ID compartment of a subject's skin to provide an improved diagnostic application of prostate cancer. In addition, the methods of the invention are particularly advantageous where the retention of a therapeutic agent in particulate form in lymph node is desired, for example, cancer metastasis.

The invention encompasses a method for administration of at least one diagnostic agent, a particulate agent for the detection or imaging of abnormal growth (e.g., tumor or metasteses) or pathogen located within or adjacent to lymphatic tissue, but always involving lymphatic vessels that provide a pathway for particulate agent to travel. The invention encompasses the delivery of particulate agent to the intradermal compartment of the human subject's skin, and thereby providing a means for entering a network of vessels (afferent and efferent) through valve-like structures. Once inside the vessels, the particulate label progresses to the sentinel nodes, regional nodes, and/or deeps nodes which constitute a large portion of the lymphatic system. In this manner, the particulate labels are made an opportunity to travel the same route as cancerous cells and human pathogens interacting for clinical benefit.

Likewise, the invention encompasses a method for administration of at least one therapeutic agent, a particulate drug, for the treatment of abnormal growth (e.g., tumor or metasteses) or pathogen located within or adjacent to lymphatic tissue but always involving lymphatic vessels that provide a pathway for particulate agent to travel. The invention also encompasses the delivery of particulate agent to the intradermal compartment of the human subject's skin and thereby providing a means for the particulate drug to enter a network of vessels (afferent and efferent) through valve-like structures. Once inside the vessels, the particulate drug progresses to the sentinel nodes, regional nodes, and/or deeps nodes which constitute a large portion of the lymphatic system. In this manner, the particulate drug has an opportunity to travel the same route as cancerous cells and human pathogens interacting for clinical benefit. The above is provided as one potential mechanism of the invention in which human benefit can occur, but is not intended to be the only means of operation.

Preferably, the agents delivered in accordance with the methods of the invention have a higher tissue bioavailability and faster onset compared to when the same agent is delivered by the conventional delivery method. Most preferably the amount of the pre-selected dose of the agent deposited in the lymphatic tissue is increased by at least 100%, at least 150%, at least 200%, at least 250%, at least 300%, at least 350% compared to when the same agent is delivered by the ID Mantoux method. In yet other preferred embodiments, the amount of the pre-selected dose of the agent deposited in the lymphatic tissue is increased by at least 100% , at least 150%, at least 200%, at least 250%, at least 300%, at least 350% compared to when the same agent is delivered to a deeper tissue compartment, e.g., SC compartment.

In one preferred embodiment, the invention provides an improved method for the diagnosis of metastasis of tumor cells, comprising: delivering a biologically active agent that is transported in vivo to the lymphatic system, tracing the biologically active agent to determine the lymphatic system draining the location, and microexamining the lymphatic system for metastasis. The invention also provides an improved method for the treatment of metastasis of tumor cells, comprising intradermally delivering a biologically active agent that is transported in vivo to the lymphatic system and is retained in the lymph node.

It is an object of the invention to provide a method for delivering a biologically active agent in particulate form, e.g., a therapeutic or diagnostic agent, to a subject comprising administering a biologically active agent into an intradermal compartment of the subject's skin, wherein the biologically active agent specifically associates with or binds to a marker of a disease or disorder. Preferably, the biologically active agent demonstrates improved biological kinetics or biological dynamics or tissue-bioavailability compared to conventional methods of delivery.

The present invention provides a method for diagnosing a disease or disorder having a specific marker, by administering a biologically active agent for said disease or disorder using the methods disclosed herein, tracing the biologically active agent and determining whether any specific binding of said agent occurs, such binding indicating the probability of said disease or disorder. The biologically active agents of the invention can be used diagnostically to, monitor the development or progression of a disease, disorder or infection as part of a clinical testing procedure to, e.g., determine the efficacy of a given treatment regimen.

The methods of the instant invention provide improved prognostic methods using specific agents (versus non-specific agents) to assess therapeutic efficacy of a treatment regimen of a disease, for example, by monitoring cellular genetic profiles in assessing gene regulation and expression over time. Traditionally, in vitro analysis of cellular genetic profiles have been used to assess gene regulation and expression over time as a tool in assessing therapeutic efficacy. Such in vitro methods have numerous shortcomings including, but not limited to, inaccuracies, the removal of cells from the body can cause the destruction of RNA and DNA thereby altering the genetic profile in the specimen, information about the morphological locus of the genetic lesion is potentially lost using ex-vivo methods, and cell differentiation and regulation may be influenced by removal from the extracellular environment in vivo. By using the methods of the present invention, intradermal administration of specific diagnostic agents capable of associating and/or binding a specific marker for a disease provides for assessment of disease as it exists in the patient. Thus, the methods taught by the present invention influence the choices of therapy available to the practitioner.

The methods of the invention are particularly useful for methods of integrated diagnosis and therapy. Accurate diagnosis of a disease is largely an unmet need for example in oncology, where few diagnostic agents indicate which therapeutic choices will succeed with any reliability. The methods of the invention provide improved methods for integrated diagnosis and therapy by administration of formulations comprising one or more diagnostic agents in combination with one or more therapeutic agents. The present invention provides methods to target diagnostic agents and therapeutic agents to a particular cell in a particular tissue. In a specific embodiment, the invention encompasses delivering formulations comprising one or more diagnostic agents in combination with one or more therapeutic agents to the ID compartment of a subject's skin such that a specific action of the diagnostic agent triggers an action, e.g., biological effect, of the therapeutic agent. The combination of targeted diagnostic delivery with targeted therapeutics delivery in accordance with the methods of the invention provides for enhanced patient care. This embodiment teaches the advantages of combining intradermal therapeutic delivery with diagnostic and/or therapeutic agents. The combination of delivering a diagnostic and a therapeutic agent to the ID compartment provides a powerful tool for improving the treatment of a disease in a subject.

In some embodiments, the invention encompasses repeated administration of one or more of the herein described particulate formulations comprising specific agents (e.g., an antibody) intradermally in the area of interest, prior to external screening process (i.e., mammography or other imaging system). Each of the administered particulate agents is then monitored during the procedure. The particulate agents may be a part of a diagnostic kit with pre-filled syringe(s) or delivery device(s). In one embodiment, monitoring of a disease, disorder or infection is carried out by repeating the method for diagnosing the disease, disorder or infection, for example, one month after initial diagnosis, six months after initial diagnosis, or one year after initial diagnosis.

The present invention also provides a method for delivering biologically active agent to a subject, in which the biologically active agent is administered to the intradermal compartment of the subject and is transported in vivo to the local lymphatic system. Thus, the biologically active agent reaches the local lymphatic system before it is excreted, degraded, or metabolized by, for example, the liver, kidneys, or spleen. In some embodiments, the biologically active agent comprises an immunoglobulin, a protein or peptide, a nucleotide, polynucleotide or nucleic acid, a ligand for a neuron receptor, an enzyme, a carbohydrate, cellular therapeutic agent, a chemospecific agent, or a combination thereof. Further, a tracer agent may be concurrently administered with the biologically active agent, or the biologically active agent itself may be labeled so that it can be traced in vivo. The tracing and examination of the tracer agent or self-labeled biologically active agent may be conducted by ex vivo flow cytometry, histological methods, or other ex vivo techniques known in the art, or in vivo using, SPECT, PET, MRI, fluorescence, luminescence, bioluminescence, optical imaging, photoacoustic imaging, RAMAN and SERS or other in vivo imaging techniques known in the art.

For agents that are administered by injection, the limits of the targeted tissue depth are controlled inter alia by the depth to which the needle or cannula outlet is inserted, the exposed height (vertical rise) of the outlet, the volume administered, and the rate of administration. Suitable parameters can be determined by persons of skill in the art without undue experimentation.

The invention encompasses administering one or more diagnostic agents employing surgical and non-surgical methods. For suitable agents, imaging via an external monitor (i.e., MRI, PET, CAT Scan, or mammography) outside of the surgical site is used. Non-surgical methods may be use for diseases which include, but are not limited to, breast cancer, lymphoma, colorectal and prostate cancer imaging and screening, early detection of rare cells indicative of a disease state, chronic diseases such as rheumatoid arthritis, and blood borne pathogens such as HIV.

Detection can be facilitated by coupling the biologically active agent to a detectable substance, or by encapsulating or entrapping the detectable substance in a particle with the agent, or by tethering the detectable substance to a structural component of the particle. For example, with a liposome, the structural component could be a phospholipid. Examples of detectable substances include various enzymes, prosthetic groups, fluorescent materials, luminescent materials, bioluminescent materials, radioactive materials, positron emitting metals, visible dyes, fluorescent dyes, radioisotopes, magnetic spin labels, and non-radioactive paramagnetic metal ions. The detectable substance may be coupled or conjugated either directly to the biologically active agent or indirectly, through an intermediate (such as, for example, a linker known in the art) using techniques known in the art. See, for example, U.S. Pat. No. 4,741,900 for metal ions which can be conjugated to antibodies for use as diagnostics according to the present invention. Such diagnosis and detection can be accomplished by coupling the biologically active agent to detectable substances including, but not limited to, various enzymes, enzymes including, but not limited to, horseradish peroxidase, alkaline phosphatase, beta-galactosidase, or acetylcholinesterase; prosthetic group complexes such as, but not limited to, streptavidin/biotin and avidinibiotin; fluorescent materials such as, but not limited to, umbelliferone, fluorescein, fluorescein isothiocyanate, rhodamine, dichlorotriazinylamine fluorescein, dansyl chloride or phycoerythrin; luminescent material such as, but not limited to, luminol; bioluminescent materials such as, but not limited to, luciferase, luciferin, and aequorin; radioactive material such as, but not limited to, bismuth (²¹³Bi), carbon (¹⁴C), chromium (⁵Cr), cobalt (⁵⁷Co), fluorine (¹⁸F), gadolinium (⁵³Gd, ¹⁵⁹Gd), gallium (⁶⁸ Ga, ⁶⁷ Ga), germanium (⁶⁸Ge), holmium (¹⁶⁶Ho), indium (115In, ¹¹³In, ¹¹²In, ¹¹¹In), iodine (¹³¹I, ¹²⁵I, ¹²³I, ¹²¹I), lanthanium (⁴⁰La), lutetium (¹⁷⁷Lu), manganese (⁵⁴Mn), molybdenum (⁹⁹Mo), palladium (¹⁰³Pd), phosphorous (³²p), praseodymium (⁴² Pr), promethium (¹⁴⁹Pm), rhenium (¹⁸⁶Re, ¹⁸⁸Re), rhodium (¹⁰⁵Rh), ruthemium (⁹⁷Ru), samarium (¹⁵³Sm), scandium (⁴⁷Sc), selenium (⁷⁵Se), strontium (⁸⁵Sr), sulfur (³⁵S), technetium (⁹⁹Tc), thallium (²⁰¹Ti), tin (¹¹³Sn, ¹¹⁷Sn), tritium (³H), xenon (¹³³Xe), ytterbium (¹⁶⁹yb, ¹⁷⁵Yb), yttrium (⁹⁰Y), zinc (⁶⁵Zn); positron emitting metals using various positron emission tomographies, and non-radioactive paramagnetic metal ions.

The invention encompasses any detection method known in the art and exemplified herein including but not limited to ex vivo or in vivo, invasive or non-invasive. Detection of the agents in accordance with the methods of the invention may be done using optical methods (e.g., time resolved and life time fluorescence spectroscopy, luminescence, or bioluminescence , chemiluminescence); flow cytometry, fluorescence in the infrared region, histological examination, ultrasonography, photoacoustics spectroscopy, Raman spectroscopy, and surface enhanced raman spectroscopy. In preferred embodiments, the examination and tracing of the location of the agent is by way of in vivo imaging. Any suitable method of in vivo imaging known in the art, including, for example, SPECT, optical imaging, photoacoustic imaging, RAMAN and SERS CAT, PET, may be used in the methods of the invention.

It will be understood in the art that the size of the subject and the imaging system used will determine the quantity of imaging moiety needed to produce diagnostic images. In the case of a radioisotope moiety, for a human subject, the quantity of radioactivity injected will normally range from about 5 to 20 millicuries of ^{99m}Tc. A radiolabeled particle will then preferentially accumulate at the location of cells which contain the specific protein. In vivo tumor imaging is described in S. W. Burchiel et al., “Immunopharmacokinetics of Radiolabeled Antibodies and Their Fragments.” (Chapter 13 in Tumor Imaging: The Radiochemical Detection of Cancer, S. W. Burchiel and B. A. Rhodes, eds., Masson Publishing Inc. (1982); which is incorporated herein by reference in its entirety.) Depending on several variables, including the type of label used and the mode of administration, the time interval following the administration for permitting the labeled molecule to preferentially concentrate at sites in the subject and for unbound labeled molecule to be cleared to background level is 6 to 48 hours or 6 to 24 hours or 6 to 12 hours. In another embodiment the time interval following administration is 5 to 20 days or 5 to 10 days.

Presence of the labeled molecule, radiolabeled or otherwise, can be detected in the subject using methods known in the art for in vivo scanning. These methods depend upon the type of label used. Skilled artisans will be able to determine the appropriate method for detecting a particular label. Methods and devices that may be used in the methods of the invention include, but are not limited to, computed tomography (CT), whole body scan such as position emission tomography (PET), magnetic resonance imaging (MRI), single photon emission computer tomography (SPECT), X-Ray, Optical (spectrophotometric) imaging and sonography.

In a specific embodiment, the biologically active agent is labeled with a radioisotope and is detected in the patient using a radiation responsive surgical instrument (Thurston et al., U.S. Pat. No. 5,441,050). In another embodiment, the biologically active agent is labeled with a fluorescent compound and is detected in the patient using a fluorescence responsive scanning instrument. In another embodiment, the biologically active agent is labeled with a positron emitting metal and is detected in the patient using positron emission-tomography. In yet another embodiment, the biologically active agent is labeled with a paramagnetic label and is detected in a patient using magnetic resonance imaging (MRI). For example, a particle of the invention itself may be labeled, its payload may be labeled, or the radioisotope may be free within the sac making up the particulate agent.

The invention encompasses in vivo imaging agents formulated and delivered in accordance to the methods of the invention. Such agents can be detected using the appropriate imaging modality. Imaging modalities include but are not limited to ultrasound, MRI, CT, PET, SPECT, Fluorescent, Chemiluminescent, Bioluminescence, X-Ray, and Photoacoustic imaging. The invention encompasses in vivo imaging of a disease or disorder using the biologically active agents and other agents disclosed herein, e.g., tracer agents, imaging agents. Once a biologically active agent is delivered to a subject, the subject may be imaged appropriately which can be during the injection, immediately after injection, and/or at an appointed times post injection. The images obtained can be continuous (real time) or episodic in manner. The images can be used to locate structures, i.e., lymph nodes, identify architectural features including obstructions, flow rate of the agent, and identify rare events.

The present invention encompasses delivering contrast agents suitable for imaging by one or more imaging techniques. Any contrast agent known in the art is contemplated within the methods and compositions of the invention. In some embodiments, the contrast agents are in particulate form and are adapted to be preferentially taken up by the lymphatic system upon administration. These contrast agents can be radiopaque materials, MRI imaging agents, ultrasound imaging agents, and any other contrast agent suitable for a device that images an animal body. Contrast agents for use in the methods of the invention are preferably nontoxic and/or non-radioactive. There are two major classes of contrast agents: paramagnetic and superparamagnetic; each of which is contemplated within the methods of the invention. Paramagnetic agents have unpaired electron spins that facilitate relaxation of nuclei, usually water protons, that can closely approach them (within 1 nm). These agents decrease both T1 and T2, are effective in uM concentrations, and can be incorporated in chelates with favorable biodistribution and toxicity profiles. Schering's patented product, GdDTPA (gadolinium diethylenetriaminepentaacetic acid), is an example of commercially available such agents.

The invention further encompasses particulate labels meeting the physical, chemical and biochemical properties described herein (e.g., size, charge, formulation, and packaging) including, but not limited to: MRI contrast agents (e.g., gadolinium, paramagnetic particles, super-paramagnetic particles), ultrasound contrast agents (e.g., microbubbles), CT contrast agents (e.g., radiolabels), X-Ray contrast agents (e.g., Iodine), PET contrast agents (e.g., any 2 photon emitter, F19, Fluoro-deoxy-glucose), Photoacoustic contrast agents (e.g., dyes, various light absorbing molecules), Optical contrast agents (e.g., Fluorescent: CY5, squaraines, near infrared dyes, i.e. indocyanine green, lanthanide fluors (e.g., Europium, Turbium).

In a particular example, microbubble ultrasound contrast agent is delivered as described herein. An ultrasound probe is positioned either at the injection site or at a regional lymph node site. Although not intending to be bound by a particular mechanism of action the contrast agent is delivered to the intradermal compartments and immediately travels through the lymphatic vessels and to the lymph node. The ultrasound probe detects the contrast agent as it passes beneath the probe. Both diagnostic flow rate and architecture information, including obstructions, can be obtained. In this embodiment, the images can be obtained continuously (real time) or in an episodic manner.

In specific embodiments, the invention encompasses a method for diagnosing a disease affecting the lymph nodes which is improved over traditional lymphography methods known in the art. The methods of the invention encompasses using ultrasound or magnetic resonance imaging. The methods of the invention encompass administering a diagnostically effective, non-toxic amount, non-radioactive contrast agent to a mammal, such that the agent is imageable with sufficient resolution through ultrasound or magnetic resonance imaging to permit visualization of intranodal architecture; permitting the contrast agent to localize in the lymph nodes; and imaging the lymph nodes of the mammal in which said contrast agent has localized with magnetic resonance imaging or ultrasound within about 30 minutes of said administration, within about 4 hours of said administration, within about 24 hours of said administration, or within about 1 month of said administration.

In some embodiments, magnetic resonance images further comprise an additional step of making sure to pre-image the subject prior to injection of the agent, e.g., contrast agent. In some embodiments, Multiple images post injection are obtained over time and compared to the pre-image. The invention encompasses methods for detection and location of lymph nodes, as well as information concerning other tissues, organs and biological entities using methods disclosed herein and known to those skilled in the art, e.g., CT, PET, SPECT, Optical (e.g., Fluorescent, Chemiluminescent) and X-Ray imaging.

5.2.1 Diseases

The methods of the invention can be used for improved treatment and diagnosis of cancers and related disorders including but not limited to, the following: Leukemias including, but not limited to, acute leukemia, acute lymphocytic leukemia, acute myelocytic leukemias such as myeloblastic, promyelocytic, myelomonocytic, monocytic, erythroleukemia leukemias and myelodysplastic syndrome, chronic leukemias such as but not limited to, chronic myelocytic (granulocytic) leukemia, chronic lymphocytic leukemia, hairy cell leukemia; polycythemia vera; lymphomas such as but not limited to Hodgkin's disease, non-Hodgkin's disease; multiple myelomas such as but not limited to smoldering multiple myeloma, nonsecretory myeloma, osteosclerotic myeloma, plasma cell leukemia, solitary plasmacytoma and extramedullary plasmacytoma; Waldenström's macroglobulinemia; monoclonal gammopathy of undetermined significance; benign monoclonal gammopathy; heavy chain disease; bone and connective tissue sarcomas such as but not limited to bone sarcoma, osteosarcoma, chondrosarcoma, Ewing's sarcoma, malignant giant cell tumor, fibrosarcoma of bone, chordoma, periosteal sarcoma, soft-tissue sarcomas, angiosarcoma (hemangiosarcoma), fibrosarcoma, Kaposi's sarcoma, leiomyosarcoma, liposarcoma, lymphangiosarcoma, neurilemmoma, rhabdomyosarcoma, synovial sarcoma; brain tumors including but not limited to, glioma, astrocytoma, brain stem glioma, ependymoma, oligodendroglioma, nonglial tumor, acoustic neurinoma, craniopharyngioma, medulloblastoma, meningioma, pineocytoma, pineoblastoma, primary brain lymphoma; breast cancer including, but not limited to, adenocarcinoma, lobular (small cell) carcinoma, intraductal carcinoma, medullary breast cancer, mucinous breast cancer, tubular breast cancer, papillary breast cancer, Paget's disease, and inflammatory breast cancer; adrenal cancer, including but not limited to, pheochromocytom and adrenocortical carcinoma; thyroid cancer such as but not limited to papillary or follicular thyroid cancer, medullary thyroid cancer and anaplastic thyroid cancer; pancreatic cancer, including but not limited to, insulinoma, gastrinoma, glucagonoma, vipoma, somatostatin-secreting tumor, and carcinoid or islet cell tumor; pituitary cancers including but not limited to, Cushing's disease, prolactin-secreting tumor, acromegaly, and diabetes insipius; eye cancers including but not limited to, ocular melanoma such as iris melanoma, choroidal melanoma, and cilliary body melanoma, and retinoblastoma; vaginal cancers, including but not limited to, squamous cell carcinoma, adenocarcinoma, and melanoma; vulvar cancer, including but not limited to, squamous cell carcinoma, melanoma, adenocarcinoma, basal cell carcinoma, sarcoma, and Paget's disease; cervical cancers including but not limited to, squamous cell carcinoma, and adenocarcinoma; uterine cancers including but not limited to, endometrial carcinoma and uterine sarcoma; ovarian cancers including but not limited to, ovarian epithelial carcinoma, borderline tumor, germ cell tumor, and stromal tumor; esophageal cancers including but not limited to, squamous cancer, adenocarcinoma, adenoid cyctic carcinoma, mucoepidermoid carcinoma, adenosquamous carcinoma, sarcoma, melanoma, plasmacytoma, verrucous carcinoma, and oat cell (small cell) carcinoma; stomach cancers including but not limited to, adenocarcinoma, ftungating (polypoid), ulcerating, superficial spreading, diffusely spreading, malignant lymphoma, liposarcoma, fibrosarcoma, and carcinosarcoma; colon cancers; rectal cancers; liver cancers including but not limited to hepatocellular carcinoma and hepatoblastoma, gallbladder cancers including but not limited to, adenocarcinoma; cholangiocarcinomas including but not limited to, pappillary, nodular, and diffuse; lung cancers including but not limited to, non-small cell lung cancer, squamous cell carcinoma (epidermoid carcinoma), adenocarcinoma, large-cell carcinoma and small-cell lung cancer; testicular cancers including but not limited to, germinal tumor, seminoma, anaplastic, classic (typical), spermatocytic, nonseminoma, embryonal carcinoma, teratoma carcinoma, choriocarcinoma (yolk-sac tumor), prostate cancers including but not limited to, adenocarcinoma, leiomyosarcoma, and rhabdomyosarcoma; penal cancers; oral cancers including but not limited to, squamous cell carcinoma; basal cancers; salivary gland cancers including but not limited to, adenocarcinoma, mucoepidermoid carcinoma, and adenoidcystic carcinoma; pharynx cancers including but not limited to, squamous cell cancer, and verrucous; skin cancers including but not limited to, basal cell carcinoma, squamous cell carcinoma and melanoma, superficial spreading melanoma, nodular melanoma, lentigo malignant melanoma, acral lentiginous melanoma; kidney cancers including but not limited to, renal cell cancer, adenocarcinoma, hypernephroma, fibrosarcoma, transitional cell cancer (renal pelvis and/or uterer); Wilms'tumor; bladder cancers including but not limited to, transitional cell carcinoma, squamous cell cancer, adenocarcinoma, carcinosarcoma. In addition, cancers include myxosarcoma, osteogenic sarcoma, endotheliosarcoma, lymphangioendotheliosarcoma, mesothelioma, synovioma, hemangioblastoma, epithelial carcinoma, cystadenocarcinoma, bronchogenic carcinoma, sweat gland carcinoma, sebaceous gland carcinoma, papillary carcinoma and papillary adenocarcinomas (for a review of such disorders, see Fishman et al., 1985, Medicine, 2d Ed., J. B. Lippincott Co., Philadelphia and Murphy et al., 1997, Informed Decisions: The Complete Book of Cancer Diagnosis, Treatment, and Recovery, Viking Penguin, Penguin Books U.S.A., Inc., United States of America). Accordingly, the methods and agents of the invention are also useful in the treatment or diagnosis of a variety of cancers or other abnormal proliferative diseases, including (but not limited to) the following: carcinoma, including that of the bladder, breast, colon, kidney, liver, lung, ovary, pancreas, stomach, cervix, thyroid and skin; including squamous cell carcinoma; hematopoietic tumors of lymphoid lineage, including leukemia, acute lymphocytic leukemia, acute lymphoblastic leukemia, B-cell lymphoma, T-cell lymphoma, Berketts lymphoma; hematopoietic tumors of myeloid lineage, including acute and chronic myelogenous leukemias and promyelocytic leukemia; tumors of mesenchymal origin, including fibrosarcoma and rhabdomyoscarcoma; other tumors, including melanoma, seminoma, tetratocarcinoma, neuroblastoma and glioma; tumors of the central and peripheral nervous system, including astrocytoma, neuroblastoma, glioma, and schwannomas; tumors of mesenchymal origin, including fibrosafcoma, rhabdomyoscarama, and osteosarcoma; and other tumors, including melanoma, xenoderma pegmentosum, keratoactanthoma, seminoma, thyroid follicular cancer and teratocarcinoma. It is also contemplated that cancers caused by aberrations in apoptosis would also be treated by the methods and compositions of the invention. Such cancers may include but not be limited to follicular lymphomas, carcinomas with p53 mutations, hormone dependent tumors of the breast, prostate and ovary, and precancerous lesions such as familial adenomatous polyposis, and myelodysplastic syndromes. In specific embodiments, malignancy or dysproliferative changes (such as metaplasias and dysplasias), or hyperproliferative disorders, are treated or diagnosed more effectively by the methods and compositions of the invention in the ovary, bladder, breast, colon, lung, skin, pancreas, or uterus. In other specific embodiments, sarcoma, melanoma, or leukemia is treated or diagnosed more effectively by the methods and compositions of the invention.

Cancers associated with the cancer antigens may be treated or diagnosed in vivo more effectively by administration of the agents of the invention, For example, but not by way of limitation, cancers associated with the following cancer antigen may be diagnosed more effectively by the methods and compositions of the invention. KS 1/4 pan-carcinoma antigen (Perez and Walker, 1990, J. Immunol. 142:32-37; Bumal, 1988, Hybridoma 7(4):407-415), ovarian carcinoma antigen (CA125) (Yu et al., 1991, Cancer Res. 51(2):48-475), prostatic acid phosphate (Tailor et al., 1990, Nucl. Acids Res. 18(1):4928), prostate specific antigen (Henttu and Vihko, 1989, Biochem. Biophys. Res. Comm. 10(2):903-910; Israeli et al., 1993, Cancer Res. 53:227-230), melanoma-associated antigen p97 (Estin et al., 1989, J. Natl. Cancer Instit. 81(6):445-44), melanoma antigen gp75 (Vijayasardahl et al., 1990, J. Exp. Med. 171(4):1375-1380), high molecular weight melanoma antigen (HMW-MAA) (Natali et al., 1987, Cancer 59:55-3; Mittelman et al, 1990, J. Clin. Invest. 86:2136-2144)), prostate specific membrane antigen, carcinoembryonic antigen (CEA) (Foon et al., 1994, Proc. Am. Soc. Clin. Oncol. 13:294), polymorphic epithelial mucin antigen, human milk fat globule antigen, Colorectal tumor-associated antigens such as: CEA, TAG-72 (Yokata et al., 1992, Cancer Res. 52:3402-3408), CO17-1A (Ragnhammar et al., 1993, Int. J. Cancer 53:751-758); GICA 19-9 (Herlyn et al., 1982, J. Clin. Immunol. 2:135), CTA-1 and LEA, Burkitt's lymphoma antigen-38.13, CD 19 (Ghetie et al., 1994, Blood 83:1329-1336), human B-lymphoma antigen-CD20 (Reff et al., 1994, Blood 83:435-445), CD33 (Sgouros et al., 1993, J. Nucl. Med. 34:422-430), melanoma specific antigens such as ganglioside GD2 (Saleh et al, 1993, J. Immunol., 151, 3390-3398), ganglioside GD3 (Shitara et al., 1993, Cancer Immunol. Immunother. 36:373-380), ganglioside GM2 (Livingston et al., 1994, J. Clin. Oncol. 12:1036-1044), ganglioside GM3 (Hoon et al., 1993, Cancer Res. 53:5244-5250), tumor-specific transplantation type of cell-surface antigen (TSTA) such as virally-induced tumor antigens including T-antigen DNA tumor viruses and envelope antigens of RNA tumor viruses, oncofetal antigen-alpha-fetoprotein such as CEA of colon, bladder tumor oncofetal antigen (Hellstrom et al., 1985, Cancer. Res. 45:2210-2188), differentiation antigen such as human lung carcinoma antigen L6, L20 (Hellstrom et al., 1986, Cancer Res. 46:3917-3923), antigens of fibrosarcoma, human leukemia T cell antigen-Gp37 (Bhattacharya-Chatterjee et al., 1988, J. of Immun. 141:1398-1403), neoglycoprotein, sphingolipids, breast cancer antigen such as EGFR (Epidermal growth factor receptor), HER2 antigen (p185^{HER2}), polymorphic epithelial mucin (PEM) (Hilkens et al., 1992, Trends in Bio. Chem. Sci. 17:359), malignant human lymphocyte antigen-APO-1 (Bernhard et al., 1989, Science 245:301-304), differentiation antigen (Feizi, 1985, Nature 314:53-57) such as I antigen found in fetal erythrocytes and primary endoderm, I (Ma) found in gastric adencarcinomas, M18 and M39 found in breast epithelium, SSEA-1 found in myeloid cells, VEP8, VEP9, Myl, VIM-D5, and D₁56-22 found in colorectal cancer, TRA-1-85 (blood group H), C14 found in colonic adenocarcinoma, F3 found in lung adenocarcinoma, AH6 found in gastric cancer, Y hapten, Le^{y} found in embryonal carcinoma cells, TL5 (blood group A), EGF receptor found in A431 cells, E₁ series (blood group B) found in pancreatic cancer, FC 10.2 found in embryonal carcinoma cells, gastric adenocarcinoma, CO-514 (blood group Le^{a}) found in adenocarcinoma, NS-10 found in adenocarcinomas, CO-43 (blood group Le^{b}), G49, EGF receptor, (blood group ALe^{b}/Le^{y}) found in colonic adenocarcinoma, 19.9 found in colon cancer, gastric cancer mucins, T₅A₇ found in myeloid cells, R₂₄ found in melanoma, 4.2, G_{D3}, D1.1, OFA-1, G_{M2}, OFA-2, G_{D2}, M1:22:25:8 found in embryonal carcinoma cells and SSEA-3, SSEA-4 found in 4-8-cell stage embryos. In another embodiment, the antigen is a T cell receptor derived peptide from a cutaneous T cell lymphoma (see Edelson, 1998, The Cancer Journal 4:62).

The biologically active agents can be used for therapeutic, diagnostic purposes to treat, detect, diagnose, or monitor infections (e.g., lymphangitis, pneumonia, slymphadenitis, streptococcus, RSV). Infectious diseases that can be treated, detected, diagnosed, or monitored by the agents of the invention are caused by infectious agents including but not limited to viruses, bacteria, fingi, protozae, and viruses.

Viral diseases that can be treated, detected, diagnosed, or monitored using the agents of the invention in conjunction with the methods of the present invention include, but are not limited to, those caused by hepatitis type A, hepatitis type B, hepatitis type C, influenza, varicella, adenovirus, herpes simplex type I (HSV-I), herpes simplex type II (HSV-II), rinderpest, rhinovirus, echovirus, rotavirus, respiratory syncytial virus, papilloma virus, papova virus, cytomegalovirus, echinovirus, arbovirus, huntavirus, coxsackie virus, mumps virus, measles virus, rubella virus, polio virus, small pox, Epstein Barr virus, human immunodeficiency virus type I (HIV-I), human immunodeficiency virus type II (HIV-II), and agents of viral diseases such as viral miningitis, encephalitis, dengue or small pox.

Bacterial diseases that can be treated, detected, diagnosed, or monitored using the agents of the invention in conjunction with the methods of the present invention, that are caused by bacteria include, but are not limited to, mycobacteria rickettsia, mycoplasma, neisseria, S. pneumonia, Borrelia burgdorferi (Lyme disease), Bacillus antracis (anthrax), tetanus, streptococcus, staphylococcus, mycobacterium, tetanus, pertissus, cholera, plague, diptheria, chlamydia, S. aureus and legionella.

Protozoal diseases that can be treated, detected, diagnosed, or monitored using the agents of the invention in conjunction with the methods of the present invention, that are caused by protozoa include, but are not limited to, leishmania, kokzidioa, trypanosoma or malaria.

Parasitic diseases that can be treated, detected, diagnosed, or monitored using the agents of the invention in conjunction with the methods of the present invention, that are caused by parasites include, but are not limited to, chlamydia and rickettsia.

5.3 Intradermal Administration of Biologically Active Agents

The invention encompasses methods for intradermal delivery of biologically active agents described and exemplified herein to the intradermal compartment of a subject's skin, preferably by directly and selectively targeting the intradermal compartment, particularly the dermal vasculature, without entirely passing through it. Once the biologically active agents for use in the methods of the invention are prepared, the agent is typically transferred to an injection device for intradermal delivery, e.g., a syringe or pen. The biologically active agents, particularly diagnostic agents may be in a commercial preparation, such as a vial or cartridge, specifically designed for intradermal injection. The biologically active agents of the invention are administered using any of the intradermal devices and methods known in the art or disclosed in WO 01/02178, published Jan. 10, 2002; and WO 02/02179, published Jan. 10, 2002, U.S. Pat. No. 6,494,865, issued Dec. 17, 2002 and U.S. Pat. No. 6,569,143 issued May 27, 2003 all of which are incorporated herein by reference in their entirety.

The actual method by which the intradermal administration of the biologically active agents is targeted to the intradermal compartment is not critical as long as it penetrates the skin of a subject to the desired targeted depth within the intradermal compartment without passing through it. In most cases, the device will penetrate the skin to a depth of about 0.5-2 mm. The invention encompasses conventional injection needles, catheters or microneedles of all known types, employed singularly or in multiple needle arrays. The dermal access means may comprise needle-less devices including ballistic injection devices. The terms “needle” and “needles” as used herein are intended to encompass all such needle-like structures with any bevel or even without a point. The term microneedles as used herein are intended to encompass structure 30 gauge and smaller, typically about 31-50 gauge when such structures are cylindrical in nature. Non-cylindrical structures encompass by the term microneedles would therefore be of comparable diameter and include pyramidal, rectangular, octagonal, wedged, and other geometrical shapes. They too may have any bevel, combination of bevels or may lack a point. The methods of the invention also include ballistic fluid injection devices, powder-jet delivery devices, piezoelectric, electromotive, electromagnetic assisted delivery devices, gas-assisted delivery devices, of which directly penetrate the skin to provide access for delivery or directly deliver agents to the targeted location within the dermal compartment.

Preferably however, the device has structural means for controlling skin penetration to the desired depth within the intradermal compartment. This is most typically accomplished by means of a widened area or hub associated with the shaft of the dermal-access means that may take the form of a backing structure or platform to which the needles are attached. The length of microneedles as dermal-access means are easily varied during the fabrication process and are routinely produced in less than 2 mm, more specifically, less than 1.5 mm, more specifically, less than 1.25 mm, length. Microneedles are also a very sharp and of a very small gauge, to further reduce pain and other sensation during the injection or infusion. They may be used in the invention as individual single-lumen microneedles or multiple microneedles may be assembled or fabricated in linear arrays or two-dimensional arrays as to increase the rate of delivery or the amount of agent delivered in a given period of time. The needle may eject its agent from the end, the side or both. Microneedles may be incorporated into a variety of devices such as holders and housings that may also serve to limit the depth of penetration. The dermal-access means of the invention may also incorporate reservoirs to contain the agent prior to delivery or pumps or other means for delivering the drug or other agent under pressure. Alternatively, the device housing the dermal-access means may be linked externally to such additional components.

The intradermal methods of administration comprise microneedle-based injection and infusion systems or any other means to accurately target the intradermal compartment. The intradermal methods of administration encompass not only microdevice-based injection means, but other delivery methods such as needle-less or needle-free ballistic injection of fluids or powders into the intradermal compartment, enhanced ionotophoresis through microdevices, and direct deposition of fluid, solids, or other dosing forms into the skin.

The invention provides a method for an improved method of delivering biologically active agents, particularly diagnostic agents into the intradermal compartment of a subject's skin comprising the steps of providing a delivery device. For example, the device includes a needle cannula having a forward needle tip and the needle cannula being in fluid communication with an agent contained in the delivery device and including a limiter portion surrounding the needle cannula and the limiter portion including a skin engaging surface, with the needle tip of the needle cannula extending from the limiter portion beyond the skin engaging surface a distance equal to approximately 0.5 mm to approximately 3.0 mm and the needle cannula having a fixed angle of orientation relative to a plane of the skin engaging surface of the limiter portion, inserting the needle tip into the skin of an animal and engaging the surface of the skin with the skin engaging surface of the limiter portion, such that the skin engaging surface of the limiter portion limits penetration of the needle cannula tip into the dermis layer of the skin of the animal, and expelling the agent from the delivery device through the needle cannula tip into the skin of the animal.

In other preferred embodiments, the invention encompass selecting an injection site on the skin of the subject, cleaning the injection site on the skin of the subject prior to expelling the biologically active agents from the delivery device into the skin of the subject. In addition, the method comprises filling the delivery device with the biologically active agents of the invention. Further, the method comprises pressing the skin engaging surface of the limiter portion against the skin of the subject and applying pressure, thereby stretching the skin of the subject, and withdrawing the needle cannula from the skin after injecting the agent. Still further, the step of inserting the forward tip into the skin is further defined by inserting the forward tip into the skin to a depth of from approximately 1.0 mm to approximately 2.0 mm, and most preferably into the skin to a depth of 1.5 mm+0.2 to 0.3 mm.

In one embodiment, the step of inserting the forward tip into the skin of the animal is further defined by inserting the forward tip into the skin at an angle being generally perpendicular to the skin within about fifteen degrees, with the angle most preferably being generally ninety degrees to the skin, within about five degrees, and the fixed angle of orientation relative to the skin engaging surface is further defined as being generally perpendicular. In the embodiment, the limiter surrounds the needle cannula, having a generally planar flat skin engaging surface. Also, the delivery device may comprise a syringe having a barrel and a plunger received within the barrel and the plunger being depressible to expel the agent from the delivery device through the forward tip of the needle cannula.

In another embodiment, expelling the biologically active agents from the delivery device is further defined by grasping the hypodermic needle with a first hand and depressing the plunger with an index finger of a second hand and expelling the agent from the delivery device by grasping the hypodermic needle with a first hand and depressing the plunger on the hypodermic needle with a thumb of a second hand, with the step of inserting the forward tip into the skin of the animal further defined by pressing the skin of the animal with the limiter. In addition, the method may further comprise the step of attaching a needle assembly to a tip of the barrel of the syringe with the needle assembly including the needle cannula and the limiter, and may comprise the step of exposing the tip of the barrel before attaching the needle assembly thereto by removing a cap from the tip of the barrel. Alternatively, the step of inserting the forward tip of the needle into the skin of the subject may be further defined by simultaneously grasping the hypodermic needle with a first hand and pressing the limiter against the skin of the animal thereby stretching the skin of the animal, and expelling the agent by depressing the plunger with an index finger of the first hand or expelling the agent by depressing the plunger with a thumb of the first hand. The method further encompasses withdrawing the forward tip of the needle cannula from the skin of the subject after the agent has been injected into the skin of the subject. Still further, the method encompasses inserting the forward tip into the skin preferably to a depth of from approximately 1.0 mm to approximately 2.0 mm, and most preferably to a depth of 1.5 mm+0.2 to 0.3 mm.

This invention encompasses the delivery methods and devices disclosed, for example, in co-pending U.S. publication no. 2005-0163711 A1, published Jul. 28, 2005, the entirety of which is incorporated herein by reference. Preferably, prior to inserting the needle cannula, an injection site upon the skin of the subject is selected and cleaned. Subsequent to selecting and cleaning the site, the forward end of the needle cannula is inserted into the skin of the subject at an angle of generally 90 degrees until the skin engaging surface contacts the skin. The skin engaging surface prevents the needle cannula from passing through the dermis layer of the skin and injecting the agent into the subcutaneous layer. While the needle cannula is inserted into the skin, the particulate agent is intradermally injected. The agent may be prefilled into the syringe, either substantially before and stored therein just prior to making the injection. Several variations of the method of performing the injection may be utilized depending upon individual preferences and syringe type. In any event, the penetration of the needle cannula is preferably no more than about 1.5 mm because the skin engaging surface prevents any further penetration.

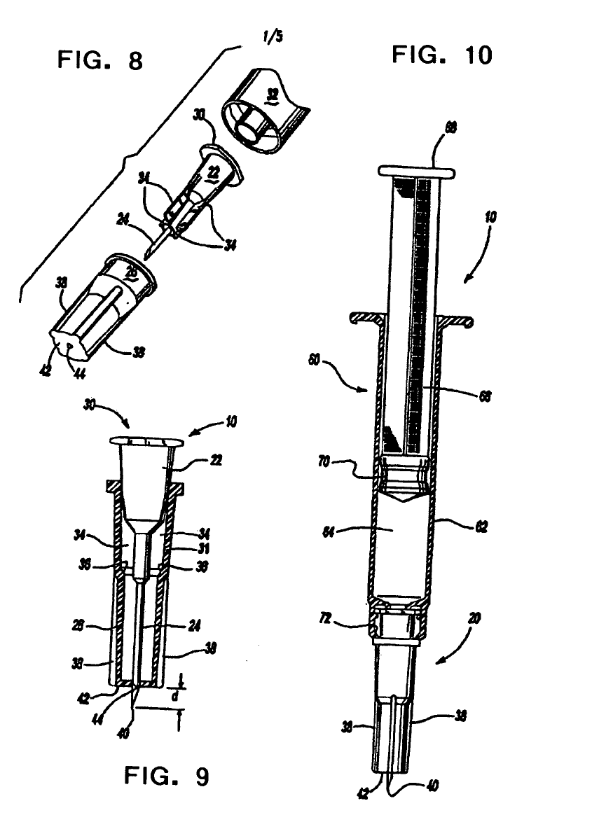

In a specific embodiment, the invention encompasses a drug delivery device as disclosed in FIG. 8-FIG. 10 illustrating an example of a drug delivery device which can be used to practice the methods of the present invention for making intradermal injections. The device 10 illustrated in FIGS. 8-10 includes a needle assembly 20 which can be attached to a syringe barrel 60. Other forms of delivery devices may be used including pens of the types disclosed in U.S. Pat. No. 5,279,586, U.S. patent application Ser. No. 09/027,607 and PCT Application No. WO 00/09135, the disclosure of which are hereby incorporated by reference in their entirety.

The needle assembly 20 includes a hub 22 that supports a needle cannula 24. The limiter 26 receives at least a portion of the hub 22 so that the limiter 26 generally surrounds the needle cannula 24 as best seen in FIG. 8.

One end 30 of the hub 22 is able to be secured to a receiver 32 of a syringe. A variety of syringe types for containing the substance to be intradermally delivered according to the present invention can be used with a needle assembly designed, with several examples being given below. The opposite end of the hub 22 preferably includes extensions 34 that are nestingly received against abutment surfaces 36 within the limiter 26. A plurality of ribs 38 preferably are provided on the limiter 26 to provide structural integrity and to facilitate handling the needle assembly 20.

By appropriately designing the size of the components, a distance “d” between a forward end or tip 40 of the needle 24 and a skin engaging surface 42 on the limiter 26 can be tightly controlled. The distance “d” preferably is in a range from approximately 0.5 mm to approximately 3.0 mm, and most preferably around 1.5 mm±0.2 mm to 0.3 mm. When the forward end 40 of the needle cannula 24 extends beyond the skin engaging surface 42 a distance within that range, an intradermal injection is ensured because the needle is unable to penetrate any further than the typical dermis layer of a subject. Typically, the outer skin layer, epidermis, has a thickness between 50-200 microns, and the dermis, the inner and thicker layer of the skin, has a thickness between 1.5-3.5 mm. Below the dermis layer is subcutaneous tissue (also sometimes referred to as the hypodermis layer) and muscle tissue, in that order.

As can be best seen in FIG. 8, the limiter 26 includes an opening 44 through which the forward end 40 of the needle cannula 24 protrudes. The dimensional relationship between the opening 44 and the forward end 40 can be controlled depending on the requirements of a particular situation. In the illustrated embodiment, the skin engaging surface 42 is generally planar or flat and continuous to provide a stable placement of the needle assembly 20 against a subject's skin. Although not specifically illustrated, it may be advantageous to have the generally planar skin engaging surface 42 include either raised portions in the form of ribs or recessed portions in the form of grooves in order to enhance stability or facilitate attachment of a needle shield to the needle tip 40. Additionally, the ribs 38 along the sides of the limiter 26 may be extended beyond the plane of the skin engaging surface 42.

Regardless of the shape or contour of the skin engaging surface 42, the preferred embodiment includes enough generally planar or flat surface area that contacts the skin to facilitate stabilizing the injector relative to the subject's skin. In the most preferred arrangement, the skin engaging surface 42 facilitates maintaining the injector in a generally perpendicular orientation relative to the skin surface and facilitates the application of pressure against the skin during injection. Thus, in the preferred embodiment, the limiter has dimension or outside diameter of at least 5 mm. The major dimension will depend upon the application and packaging limitations, but a convenient diameter is less than 15 mm or more preferably 11-12 mm.

It is important to note that although FIG. 8 and 9 illustrate a two-piece assembly where the hub 22 is made separate from the limiter 26, a device for use in connection with the invention is not limited to such an arrangement. Forming the hub 22 and limiter 26 integrally from a single piece of plastic material is an alternative to the example shown in FIGS. 8 and 9. Additionally, it is possible to adhesively or otherwise secure the hub 22 to the limiter 26 in the position illustrated in FIG. 10 so that the needle assembly 20 becomes a single piece unit upon assembly.

Having a hub 22 and limiter 26 provides the advantage of making an intradermal needle practical to manufacture. The preferred needle size is a small Gauge hypodermic needle, commonly known as a 30 Gauge or 31 Gauge needle. Having such a small diameter needle presents a challenge to make a needle short enough to prevent undue penetration beyond the dermis layer of the skin. The limiter 26 and the hub 22 facilitate utilizing a needle 24 that has an overall length that is much greater than the effective length of the needle which penetrates the individual's tissue during an injection. With a needle assembly designed in accordance herewith, manufacturing is enhanced because larger length needles can be handled during the manufacturing and assembly processes while still obtaining the advantages of having a short needle for purposes of completing an intradermal injection.

FIG. 10 illustrates the needle assembly 20 secured to a drug container such as a syringe 60 to form the device 10. A generally cylindrical syringe body 62 can be made of plastic or glass as is known in the art. The syringe body 62 provides a reservoir 64 for containing the substance to be administered during an injection. A plunger rod 66 has a manual activation flange 68 at one end with a stopper 70 at an opposite end as known in the art. Manual movement of the plunger rod 66 through the reservoir 64 forces the substance within the reservoir 64 to be expelled out of the end 40 of the needle as desired.

The hub 22 can be secured to the syringe body 62 in a variety of known manners. In one example, an interference fit is provided between the interior of the hub 22 and the exterior of the outlet port portion 72 of the syringe body 62. In another example, a conventional Luer fit arrangement is provided to secure the hub 22 on the end of the syringe 60. As can be appreciated from FIG. 10, such needle assembly design is readily adaptable to a wide variety of conventional syringe styles.

This invention provides an intradermal needle injector that is adaptable to be used with a variety of syringe types. Therefore, this invention provides the significant advantage of facilitating manufacture and assembly of intradermal needles on a mass production scale in an economical fashion.

Prior to inserting the needle cannula 24, an injection site upon the skin of a subject is selected and cleaned. Subsequent to selecting and cleaning the site, the forward end 40 of the needle cannula 24 is inserted into the skin at an angle of generally 90 degrees until the skin engaging surface 42 contacts the skin. The skin engaging surface 42 prevents the needle cannula 42 from passing through the dermis layer of the skin and injecting the substance into the subcutaneous layer.

When the needle cannula 42 is inserted into the skin, the substance is intradermally injected. The substance may be prefilled into the syringe 60, either substantially before and stored therein or just prior to making the injection. Several variations of the method of performing the injection may be utilized depending upon individual preferences and syringe type. In any event, the penetration of the needle cannula 42 is most preferably no more than about 1.5 mm because the skin engaging surface 42 prevents any further penetration.

Also, during the administration of an intradermal injection, the forward end 40 of the needle cannula 42 is embedded in the dermis layer of the skin which results in a reasonable amount of back pressure during the injection of the substance. This back pressure could be on the order of 76 psi. In order to reach this pressure with a minimal amount of force having to be applied by the user to the plunger rod 66 of the syringe, a syringe barrel 60 with a small inside diameter is preferred such as 0.183″ (4.65 mm) or less. The method of this invention thus includes selecting a syringe for injection having an inside diameter of sufficient width to generate a force sufficient to overcome the back pressure of the dermis layer when the substance is expelled from the syringe to make the injection.

In addition, since intradermal injections are sometimes carried out with small volumes of the substance to be injected, i.e., on the order of no more than 0.5 ml, and preferably around 0.1 ml, a syringe barrel 60 with a small inside diameter is preferred to minimize dead space which could result in wasted substance captured between the stopper 70 and the shoulder of the syringe after the injection is completed. Also, when injecting small volumes of substance, on the order of 0.1 ml, a syringe barrel with a small inside diameter is preferred to minimize air head space between the level of the substance and the stopper 70 during process of inserting the stopper. Further, the small inside diameter enhances the ability to inspect and visualize the volume of the substance within the barrel of the syringe.

The syringe 60 may be grasped with one hand and the plunger 66 depressed with the forefinger 114 of a second hand. Alternatively, the plunger 66 may be depressed by the thumb of the second hand while the syringe 60 is held by the first hand. In each of these variations, the skin is depressed and stretched by the skin engaging surface 42 on the limiter 26, and the skin is not contacted by either hand.

An additional variation has proven effective for administering the intradermal injection of the present invention. This variation includes gripping the syringe 60 with the same hand that is used to depress the plunger 66. The syringe 60 may be gripped with one hand while the plunger is simultaneously depressed with the thumb of the same hand. This variation includes stretching the skin with the second hand while the injection is being made. Alternatively, the grip may be reversed and the plunger is depressed by the forefinger of the first hand while the skin is being stretched by the second hand. However, it is believed that this manual stretching of the skin is optional and merely represents a variation of the standard technique.

In each of the variations described above, the needle cannula 24 is inserted only about 1.5 mm into the skin. Subsequent to administering the injection, the needle cannula 24 is withdrawn from the skin and the syringe 60 and needle assembly 20 are disposed of in an appropriate manner.

It has been found that certain features of the intradermal administration methods provide clinically useful PK/PD and dose accuracy. For example, it has been found that placement of the needle outlet within the skin significantly affects PK/PD parameters. The outlet of a conventional or standard gauge needle with a bevel has a relatively large exposed height (the vertical rise of the outlet). Although the needle tip may be placed at the desired depth within the intradermal compartment, the large exposed height of the needle outlet causes the delivered agent to be deposited at a much shallower depth nearer to the skin surface. As a result, the agent tends to effuse out of the skin due to backpressure exerted by the skin itself and to pressure built up from accumulating fluid from the injection or infusion and to leak into the lower pressure regions of the skin, such as the subcutaneous tissue. That is, at a greater depth a needle outlet with a greater exposed height will still seal efficiently where as an outlet with the same exposed height will not seal efficiently when placed in a shallower depth within the intradermal compartment. Typically, the exposed height of the needle outlet will be from 0 to about 1 mm. A needle outlet with an exposed height of 0 mm has no bevel and is at the tip of the needle. In this case, the depth of the outlet is the same as the depth of penetration of the needle. A needle outlet that is either formed by a bevel or by an opening through the side of the needle has a measurable exposed height. It is understood that a single needle may have more than one opening or outlets suitable for delivery of agents of the invention to the dermal compartment.

It has also been found that by controlling the pressure of injection or infusion the high backpressure exerted during ID administration can be overcome. By placing a constant pressure directly on the liquid interface a more constant delivery rate can be achieved, which may optimize absorption and obtain the improved pharmacokinetics. Delivery rate and volume can also be controlled to prevent the formation of wheals at the site of delivery and to prevent backpressure from pushing the dermal-access means out of the skin and/or into the subcutaneous region. The appropriate delivery rates and volumes to obtain these effects may be determined experimentally using only ordinary skill. Increased spacing between multiple needles allows broader fluid distribution and increased rates of delivery or larger fluid volumes.

The administration methods useful for carrying out the invention include both bolus and infusion delivery of the biologically active agents of the invention to humans or animals subjects. A bolus dose is a single dose delivered in a single volume unit over a relatively brief period of time, typically less than about 10 minutes. Infusion administration comprises administering a fluid at a selected rate that may be constant or variable, over a relatively more extended time period, typically greater than about 10 minutes. To deliver an agent of the invention, the dermal-access means is placed adjacent to the skin of a subject providing directly targeted access within the intradermal compartment and the agent or agents are delivered or administered into the intradermal compartment. The dermal-access means may be connected to a reservoir containing the agent or agents to be delivered.

Delivery from the reservoir into the intradermal compartment may occur either passively, without application of the external pressure or other driving means to the agent or agents to be delivered, and/or actively, with the application of pressure or other driving means. Examples of preferred pressure generating means include pumps, syringes, pens, elastomer membranes, gas pressure, piezoelectric, electromotive, electromagnetic or osmotic pumping, or Belleville springs or washers or combinations thereof. If desired, the rate of delivery of the agent may be variably controlled by the pressure-generating means.

The methods of the invention encompass controlled delivery of the biologically active agent using algorithms having logic components that include physiologic models, rules based models or moving average methods, therapy pharmacokinetic models, monitoring signal processing algorithms, predictive control models, or combinations thereof.

### 6. EXAMPLES

The following examples are illustrative, and should not be viewed as limiting the scope of the present invention. Reasonable variations, such as those that occur to reasonable artisan, can be made herein without departing from the scope of the invention.

6.1 Payload Assay

6.1.1 Pi Assay

To determine the phospholipid concentration of liposomes, 10 μl of each liposome solution was placed in a 16×100 mm glass tube along with 0.4 ml of 10 N sulfuric acid. A set of phosphate standards was prepared from a 0.645 mM stock (Sigma P3869) which was also assayed in glass tubes alongside the liposome samples. Samples, standards and blank tubes were run in duplicate. All tubes were placed in a heating block and digested for 30 minutes at 180° C. Tubes were then removed from the heating block and cooled for 5 minutes before adding 100 μl of 9% hydrogen peroxide. All tubes were then again heated for 30 minutes at 180° C. Tubes were removed from the heating block and allowed to cool for five minutes before adding 0.4 ml of ammonium molybdate reagent (0.2% w/v) with vortexing. Next, 200 μl of Fiske and Subbarow reducer was added to each tube with vortexing. Tubes were heated for 15 minutes at 110° C. to develop color, then removed from heat and allowed to cool to room temperature. All standards, samples and blanks were read at 820 nm on a spectrophotometer. Using the known standard values, a curve was generated from which phosphate concentration values can be calculated for each of the liposome samples. Phosphate concentration values are expressed as μmoles Pi/mL. Liposome particle performance was compared in vivo after standardizing each test batch of particulate agents by phosphorus content.

6.1.2 Low Molecular Weight Substance-Payload Determination

Sulfa rhodamine-B sodium salt (“SRB”) was used to determine payload capacity for a low molecular weight substance. To determine SRB dye payload in liposomes, liposome formulations were adjusted to a phosphate concentration of 1 μmole Pi/ml in liposome storage buffer. Each formulation was then diluted 1:20 with 5% Triton X-100 to release encapsulated dye. A standard curve was produced by preparing known concentrations of SRB dye in 5% Triton X-100. All samples and standards were then read at 565 nm on a spectrophotometer. SRB dye payload molar concentrations were then calculated from the known standard curve. Using this procedure, the payload for SRB was determined to be about 10.956 nmoles per 50 nmoles of phosphate. Calculating further, these SRB results demonstrate that a preferred particle should be 20% detectable marker on a molar basis, whereby the other 80% may be molecules other than detector that form the particle structure, outer wall, bilayer, outer membrane or shell. From an in vivo study using several 5× dilutions of the above particles, it is estimated that a functional particulate label may also contain 4% detector and 0.8% detector on a molar basis. Functional Dye/Pi values are provided for a range of liposome preparations in FIG. 3. Larger values translate into more detector encapsulated.

6.1.3 High Molecular Weight Substance-Payload Determination

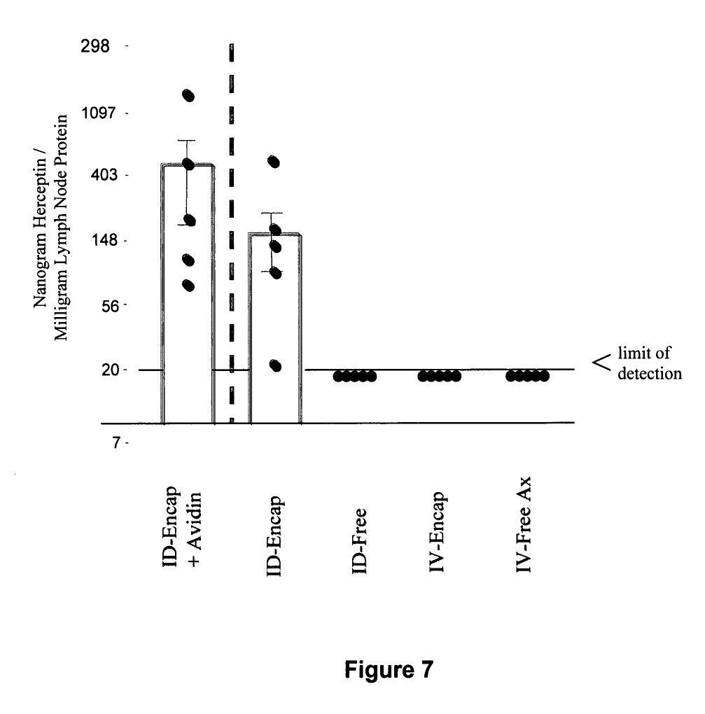

Herceptin monoclonal antibody was used for the determination of payload capacity for a high molecular weight substance. Approximate amount of monoclonal antibody encapsulated in liposomes was determined by polyacrylamide gel electrophoresis. A liposome sample of known phosphate concentration was loaded on a gel alongside the reference lanes of known quantities of monoclonal antibody. After running the gel and staining with Coomassie blue, the intensity of the protein bands for the liposome encapsulated antibody was compared to the lanes containing known amount of antibody. From this comparison of band intensities, an approximate amount of liposome encapsulated antibody could be approximated within a 5 μg range. Using this approximate value and known phosphate concentration of the antibody loaded liposomes, the payload for herceptin antibody was determined to be about 0.024 nmoles per 50 nmoles of phosphate. In the Herceptin example, the therapeutic agent represents about 0.07% on a per weight basis. The synergy resulting from the combination of ID delivery and particulate-Herceptin is demonstrated in FIG. 7. The Herceptin encapsulation values described above were critical to achieving the lymph node levels of Herceptin. In contrast, packaging analysis performed on carboplatin liposomes determined the carboplatin drug to represent about 7% of the total particle on a per weight basis.

6.2 Lymph Node Penetration Assay

6.2.1 Materials

Avidin was purchased from Sigma, with the concentration at 10 mg/ml. Avidin was hydrated with injectable saline. A 50 μl aliquot, at a dose of 250 μg, was delivered as described.

SRB was purchased from Sigma (Cat.# S9012). SRB was selected for its high solubility, and due to the fact that it is readily visible to the naked eye and has fluorescent properties.

6.2.2 LIPOSOMES

An LUV process was used to make the SRB-liposomes. Two basic formulations were developed—hi and low PE-biotin content. The low biotin formulation was used for further experiments. The liposome formulation contained the following: DSPC (94.0 mg; 119.0 μmoles); DSPG (10.3 mg; 13.2 μmoles); Chol (50.9 mg; 132.0 μmoles); and Diol Biotin (2.6 mg; 2.64 μmoles). The mixture was brought to about 20 ml with chloroform and methanol at a 10:1 ratio. Organic solvent was removed at 45° C. A thin film on a rotovap flask was hydrated in 18 ml of 0.1M SRB prepared in buffer (1 mM EDTA, 0.2% NaN₃, pH=8.0). The lipid dye suspension was then extruded through a series of nucleopore membranes (5.0. 1.0, 0.8, 0.6, 0.4, 0.2, 0.1, 0.05 micron). Approximately 5 ml was saved after the 0.2, 0.1 and 0.05 micron membranes. Each of the 5 ml preps was centrifuged, and pellets washed with osmotically adjusted (physiological 310 mOSM) buffer. After the free SRB was washed away, the final liposome pellets were resuspended in the same buffer to the final volume of 1 ml.

The final phospholipid concentrations were as follows: 0.2 micron membrane material=11.06 μmol/ml; 0.1 micron membrane material=12.93 μmol/ml; and 0.05 micron membrane material=8.66 μmol/ml. Particle distribution encompassed particles of 30 nm to 300 nm in size.

An equal amount, about 330 μl, from each of the three preps above was combined for the initial animal trials. Accordingly, the final concentration of phospholipids used in delivery was 10.88 μmol/ml, and a single 50 μl dose was administered.

6.2.3 Delivery

CD female rats weighing about 250 grams were obtained from Charles River. Prior to the delivery of liposomes, animals were anesthetized with isoflurane, and 100 μl of the Ketamine,Xylazine and Ace cocktail (Ketamine 100 mg/ml−30 ml; Xylazine 100 mg/ml−1 ml; and Ace 10 mg/ml−10 ml; resulting total volume=41 ml) was injected.

Once sufficiently anesthetized, the CD rat was laid in a position of lateral recumbency. The intial 50 μl intradermal injection of aggregating agent (avidin) was administered over the rib cage, medial to the dorsal and ventral aspects and medial to the floating ribs to the rear and sub-scapular region. Thirty minutes after injection of the intradermal aggregating agent, a 50 μl intradermal injection of liposomes was administered over the floating ribs medial to the dorsal and ventral aspects. Briefly, three animals respectfully received: 1) avidin intradermally, followed 30 minutes later by liposomes intradernally, with a 1 cm physical spacing from the site of avidin injection; 2) avidin subcutaneously, followed 30 minutes later by liposomes subcutaneously, with a 1 cm physical spacing from the site of avidin injection; and 3) avidin intravenously, followed 30 minutes later by liposomes intravenously, with a 1 cm physical spacing from the site of avidin injection. As a control, free SRB (without encapsulation in liposomes) was administered intradermally to a CD female rat. Nodes were collected for observation.

6.2.4 Results

The nodes harvested 24-hours post-intradermal delivery of the aggregating agent, avidin, and the subsequent intradermal delivery of SRB liposomes manifested a definitive pattern of aggregates across the afferent node surface, a bespectacled fuchsia appearance. The nodes harvested 24-hours post-subcutaneous delivery of the aggregating agent, avidin, and the subsequent subcutaneous delivery of SRB liposomes manifested no evidence of liposomal aggregation or accumulation. The nodes harvested 24-hours post-intravenous delivery of the aggregating agent, avidin, and the subsequent intravenous delivery of SRB liposomes manifested no evidence of liposomal aggregation or accumulation. See Table 3 below.

TABLE 3
Node Penetration Assay

| | Visible Aggregation/ |
|---|---|
| | Accumulation in |
| Injection Route | Axillary Node |
| Intradermal injection of encapsulated SRB | Yes |
| Subcutaneous injection f encapsulated SRB | No |
| Intravenous injection of encapsulated SRB | No |
| Intradermal Control injection free SRB | No |

Free SRB liposomes (not involving encapsulation) were delivered intradermally as a control. The result was no SRB color retention in the node. This result indicates that intradermal injection of the free dye itself is not a contributing factor for the observed injection. Instead, the results showed that intradermal delivery, in combination with avidin-biotinylated liposome interaction (e.g., aggregation of delivered liposomes), caused the retention of the liposomes in the node. The aggregation of the liposome particle constitutes a transformation leading to an increase in aggregate particle size.

6.3 Solution Stability

All liposome preparations were appraised for their ability to stay in suspension when placed in a known “Storage Buffer.” Stable reagents provides the greatest flexibility in a clinical environment and require less handling by the clinician. Liposomes having a tendency to settle out before delivery are unpredictable reagents that may diffuse unevenly from the injection site and ultimately show poor penetration in the lymph node. Large individual particles and smaller particles that aggregate in vitro can both settle out and cause problems in therapeutic or diagnostic applications. Stability was determined by visual inspection of stocks ranging from 1 to 15 μmole Pi/ml. Solutions were stored upright in 15 ml centrifuge tubes at 2-8° C. Formulations were inspected at 24 hours for signs of settling followed by periodic checks. Liposomes batches were monitored out past 90 days. The stability properties of various liposome preparations are shown in FIG. 3. The preparations in FIG. 3 with an average particle size of 225 nm and 277 nm have the preferred stability. The preparations with an average size of 605 nm and 557 nm have the next preferred stability.

6.4 Injection Site Diffusion Assay

Once sufficiently anesthetized, the Yorkshire swine was laid in a position of lateral recumbency and maintained on Isoflurane. The flank area of the swine was shaved and cleaned in preparation for intradermal delivery. The Liposome preps were delivered at lumolePi/mL concentration. Each delivery was a 200 μL bolus intradermal injection via either a 34-gauge lmm needle or 34-gauge 1.5 mm needle. Tissue sections of the injection sites were excised at various times ranging from 1-72 hours. The tissue sections were snap-frozen in a dry ice/2-Methylbutane bath before a section was taken and photographed with a Nikon SMZ-U sectioning scope. Each section depicted the distribution of the liposome prep in the intradermal/subcutaneous layers.

6.5 Effects of Particle Size and Needle Lengh

6.5.1 Determination of Particle Size

Several techniques suitable for determining the size of liposome particles are available. One example is dynamic or quasi-elastic light scattering. The light scattering method provides a mean diameter and distribution of particles. The technique can also distinguish whether the particle population is uniform (having one versus several peaks). Another example is electron microscopy—a technique that does not allow for good distribution characterization and may induce some unwanted changes to particle size during fixation and staining steps.

Light scattering techniques were used to collect data for certain embodiments of this invention. Values were obtained from a Beckman-Coulter N4, Beckman/Coulter N5, Malvern Zetasizer Nano and the comparable Brookhaven Instrument. Particle size (diameter) was described in nanometers (nm). The highest and lowest values were recorded for each batch of liposomes. An average particle size was calculated for all liposome batches. In addition, the Beckman and Brookhaven Instruments provide scatter intensity values for batches having particles less than 3 microns. The percentages are assigned to set “Bins.”The intensity values provide a more detailed view into particle distribution. The first column in FIG. 3 provides the size values for six batches of liposomes. All six batches were prepared with the same lipid composition. All contain SRB dye and have a similar zeta potential.

6.5.2 Particle Size and Needle Length

Using the site diffusion procedure described in Section 6.4, the diffusion of two sets of biotin liposome particles encapsulating SRB, according to different needle lengths, was investigated. In one set, liposome particles having an average diameter of about 557 nm (1000 nm filter) were injected into swine using a 1 mm or 1.5 mm 34-gauge needle. In another set, liposome particles having an average diameter of about 225 nm (100 nm filter) were injected into swine using a 1 mm or 1.5 mm 34-gauge needle. For both sets, liposomes injected using the 1 mm needle were mostly retained in intradermal space. When the 1.5 mm needle was used, while at least part of the liposomes having 557 nm average diameter was retained in the intradermal space, those with 225 nm average diameter mostly drained to subcutaneous space. In general, it was observed that particles of smaller size drained from intradermal and subcutaneous tissue faster than those of larger size. The results suggest that, where intradermal delivery to target the local draining lymph node is desired, particles smaller than about 150 nm in diameter are preferably delivered at a depth in ID space more shallow than 1.5 mm.

6.6 Effects of Particle Charge on Node Penetration

6.6.1 Determination of Particle Charge

Particle charge measurements were made by measuring zeta potentials. The measurements were performed on a Malvern Zetasizer Nano and a comparable Brookhaven Instrument. These instruments directly measure the electrophoretic mobility of the particles. The dispersant was water, and electrophoresis was conducted at 25 degrees centigrade. Zeta potential was calculated from the mobility data.

Light scattering principles are leveraged in the determination of Zeta Potential. In general, particles move in an electric field of known strength across the interference pattern of two laser beams, producing scattered light that oscillates in time in a manner characteristic of particle speed. The units are millivolts (mV), and the number can be positive or negative.

6.6.2 Effects of Particle Charge

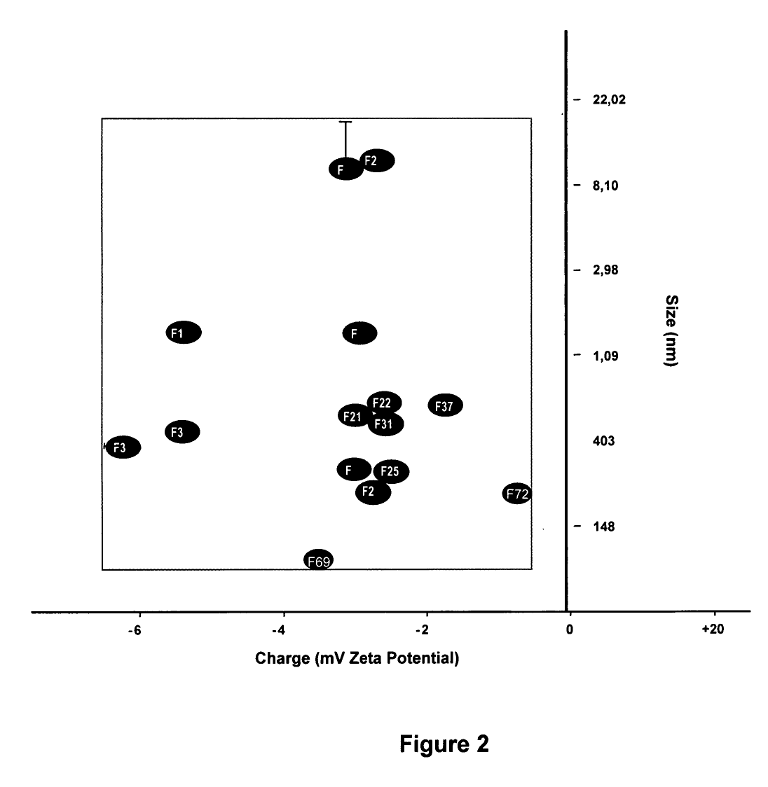

SRB-Liposome particles of varying sizes were injected into female CD rats using the methods described herein. Particles were plotted according to size and charge (FIG. 2). The particles shown in the box reached the lymph node, with varying efficiency. As shown in the figure, particles with a size from about 80 nm to about 16,000 nm in diameter exhibited some level of the desirable properties. With regard to charge, particles having the charge of from about −5 mV to about −60 mV exhibited desirable properties. Particles smaller than 150 nm in diameter reached the node, but may contain less payload and have a propensity to penetrate the subcutaneous space. Results achieved with liposomes containing HERCEPTIN and separately Carboplatin show that a functional particle can have a zeta potential value from −1 to −80 millivolts. This zeta potential range should be particularly true for particles having a 10 nm to 300 nm diameter.

6.7 Characterization of In Vivo Transformation

The nature of in vivo transformation of the delivered particles can be assessed by characterizing the particles recovered from the test animals subsequent to the injection. Exemplary methods of characterizing the recovered particles are described below.

6.7.1 Characterizing Particles Recovered from Lymph Node

One strategy for identifying particles that reach the blood stream involves a post delivery perfusion of the immediate draining lymph node. In this analysis, particles are initially trapped by the node and subsequently recovered for size analyses. Specifically, the axillary lymph nodes from CD 1 female rats are extracted after delivery. Lymph nodes are perfused with a buffer that is compatible with the liposomes (Storage Buffer) using a 31-gauge needle attached to a 1 ml syringe. The perfusate is then centrifuged at low speed to remove any cells and larger materials from suspension. The supernatant from the low speed centrifugation is then removed and subjected to a high-speed (40 k) centrifugation in an ultracentrifuge to pellet any liposomes present. Liposome pellets are resuspended in a buffer that is compatible with the liposomes and analytical method. Size analysis may include microscopic examination or light-scattering techniques described in other sections.

6.7.2 Characterizing Particles Recovered by a Cannulated Thoracic Duct

Another strategy for identifying particles that reach the blood stream involves cannulation of the thoracic and mesenteric lymph duct. Care must be taken in the interpretation of such data since particles may reach these major ducts bypassing immediate draining lymph node. While many species can be used for these studies, the rat is the most documented. In the rat, the thoracic duct arises about 2 cm before the diaphragm at about the level of the left adrenal vein. It lies beside and slightly beneath the dorsal aorta on the left, side and passes anteriorly through the diaphragm into the thorax and cervical regions. Posteriorly, it passes underneath the aorta as the broad cisterna chyli and gives rise to the mesenteric lymph duct.

The thoracic duct is a delicate structure and often nearly transparent. In certain rat strains, the thoracic duct is difficult to see, and visualization requires a dissecting microscope. Often, 50-100 μgl of a 1% w/v Evans Blue Dye is delivered into the middle members of the mesenteric group of lymph nodes that lay along side the ilio-colonic junction. The injection of die improves the visibility of mesenteric and thoracic ducts.

Particles that reach these major ducts will likely be smaller than the passageways within the node. Lymph collected at this location can be examined microscopically and by light scattering techniques to determine particle size.

6.8 Particle Properties and Performance

Node penetration and site diffusion assays described herein were performed by intradermally delivering liposome particles with varying sizes, payloads, and solution properties. The results are summarized in FIG. 3, columns 2 and 6. As can be seen from the figure, particles with a broad range of size and payload can be successfully used in connection with the methods of the invention. Certain particle subpopulations within FIG. 3 are preferred. The smallest two preparations with an average particle diameter of 225 and 277 provided the most consistent drainage of the injection site. The data shown in FIG. 3 was generated with liposomes prepared with 50.9 mg cholesterol, 10.3 mg DSPG and 94 mg DSPC. The zeta potential of these particles spanned from −29.29 mV to −31.27 mV and all particles contained SRB dye.

6.9 Remote Visualization of Liposome Drainage and Resolution of Lymph Node Architecture

From the first studies performed in Yorkshire Swine, drainage could be observed remotely (outside of body). Drainage of liposomes could be seen immediately with a lipid formula comprising DSPG, DSPC and Cholesterol. Both particle preps F4 and F9 could be seen draining from the injection site and accumulating around the Yorkshire midline. Since SRB has fluorescent properties, the drainage could be viewed with a mineral lamp as well. The first experiments conducted in Yorkshire Swine involved a 200 μl injection volume at 1-micromolar-phosphate (liposome solution) delivered with a 31 g×1.5 mm needle. Drainage (color) faded substantially by one hour. At 75 minutes post delivery, a dual-band mineral lamp (254/366 nm) was passed over both administration sites in an attempt to illuminate residual liposome reagent. The 5-micron site was indisputably fluorescent. The site receiving the 0.2-micron liposome possessed only trace fluorescence, fμuirther evidencing that smaller particles evacuate the injection site with better efficiency. Overall, illumination was an option but not a requirement to observe drainage from outside the body. No special glasses or other instrumentation was necessary to observe the drainage in visible or fluorescent light scenarios.

In the CD Rat Model, the focus was on vessels leading to the node (afferent) and node specific architecture. These studies showed that different liposome formulations and processing could affect penetration and migration in the node. For example, liposome formula F26, which contained cholesterol, DSPC and low DSPG, infiltrated the axillary node evenly, filling the organ evenly. As a result, F26 like formulations permitted a consistently high amount of SRB agent to be delivered to the node. Liposome formula F35, which contained high concentrations of DSPG filled the node in a wave like manner, whereas areas of the node were intense with SRB color, others areas of the node were clear without SRB-vesicle. Node infiltration was almost completely eliminated by simultaneously raising the size of the particle to a less preferred diameter (˜1000 nm) and by the addition of DSPA (F39 formulation). Therefore, developing particles capable of carrying high concentrations of drug and/or detector to the node involves balancing the lipid components with overall particle size and charge. These discoveries allow for better tissue diagnostics and treatments. The ability to diagnose and treat compartments without dissection of the tissue and without attaching special targeting ligands such as antibodies is unique. These results demonstrate that combining the liposome constituents and special processing with targeting ligands can lead to greater enhancements. Replacing the SRB dye with NIR dyes such as Cardiogreen, radio nucleotide or ultrasound contrast agents, can further enhance the remote (extranodal/ex vivo) analysis of lymph nodes.

6.10 Detection of Encapsulated Platinum Based Drugs Via Mass Spectrophotometry Assay

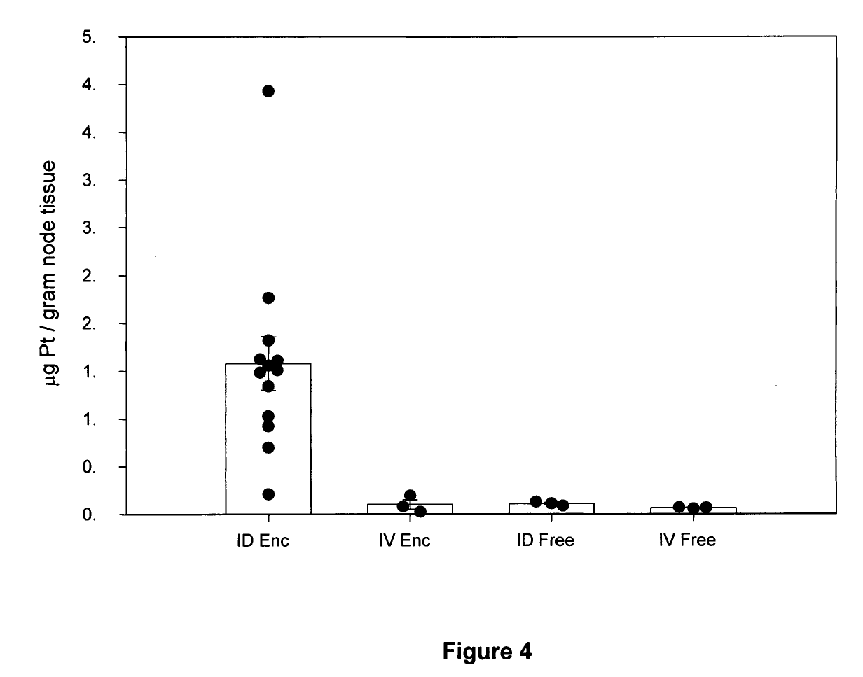

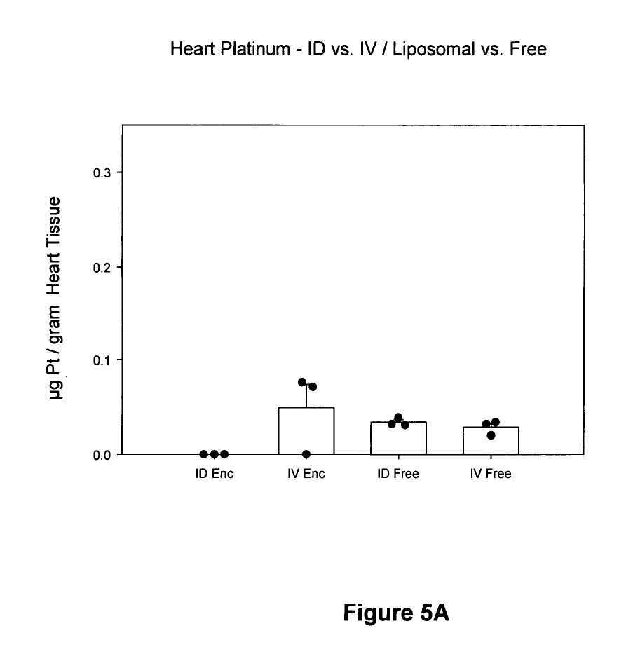

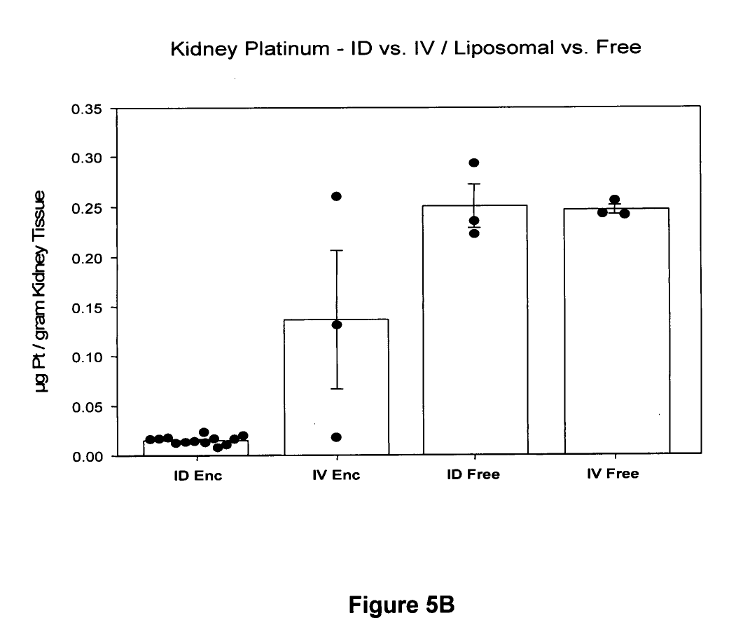

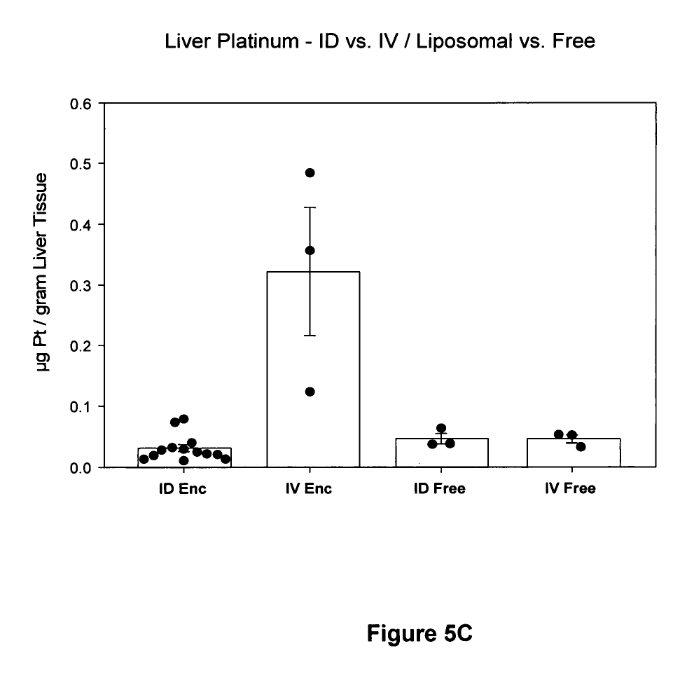

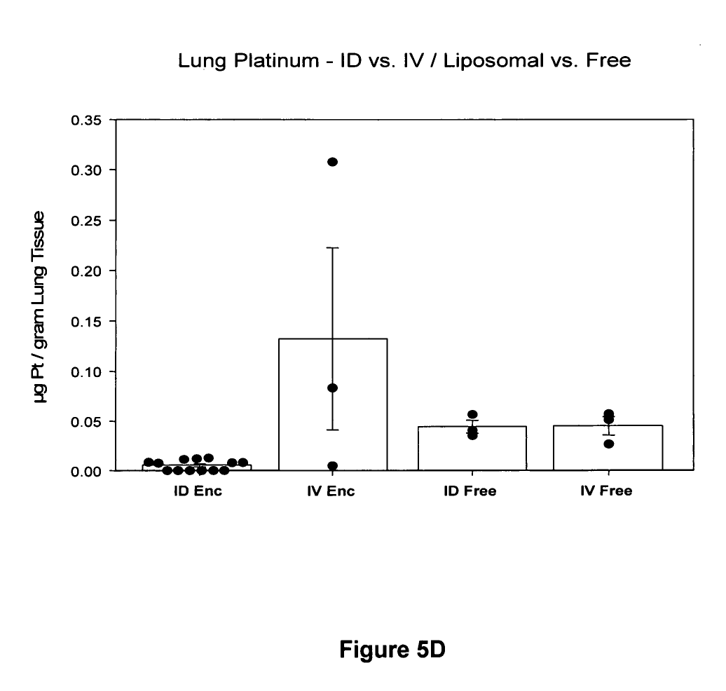

Carboplatin is a anti-cancer chemotoxic platinum (Pt) based drug that was encapsulated via pegylated liposome formulations, F58 and F60, which both contain 31.9 mg of Cholesterol, 95.8 mg of HSPC and 31.9 mg PE-PEG 2000 and are approximately 217 nm in size. Both free carboplatin and encapsulated carboplatin were administered to the CD rat model via ID and IV delivery. Once sufficiently anesthetized, the CD rats were laid in a position of lateral recumbency. Two intradermal 50 uL injections of encapsulated or free Carboplatin were administered, one over the rib cage, medial to the dorsal and ventral aspects and medial to the floating ribs to the rear and sub-scapular region and the second proximal to the palpable pelvic bone. The procedure was repeated bilaterally to equal a 200 uL dose of encapsulated or free Carboplatin given in four intradermal injections. The intradermal injections were performed with a 34-gauge Imm microneedle. Once the CD rats were sufficiently anesthetized, the intravenous deliveries of free and encapsulated Carboplatin were administered via a 200 uL bolus injection to the jugular vein. The encapsulated Carboplatin was administered at a standard injection concentration of 10 umole/ml, thereby delivering approximately 77 ug of Pt to the rodents. The free Carboplatin was administered at a comparable concentration. Twenty-four hours after administration, the lymph node and organ tissue were collected and placed in 15 mL graduated polypropylene centrifuge tubes. The liver tissue was divided into 2 tubes. The following amount of Baker Ultrex concentrated nitric acid was added to each: 1 mL to organs, 333 uL to the nodal tissue, 2 mL to each liver half. The tubes were capped and placed in a hot water bath at 90° C. for 30 minutes. After cooling to room temperature, the following amount of Baker Ultex concentrated hydrochloric acid was added to each: 1 ml to organs, 333 ul to the nodal tissue, 2 ml to each liver half. The tubes were capped and placed in a hot water bath at 90° C. for 10 minutes. After cooling to room temperature, each tube was diluted with deionized water, to a final volume of 15 mL for organs, 5 ml for nodal tissue and 30 ml for each liver half. 7.5 ml for each tube containing the liver half sample were combined in a separate tube for analysis. Each sample was submitted to an outside vendor, and assayed for Pt by ICP-MS; the results are shown in FIG. 4 and FIG. 5A-FIG. 5D.

FIG. 4 shows that ID administration of encapsulated carboplatin results in significantly higher concentrations of Pt in the lymph node compared to IV administration or ID administration of free carboplatin. The mean lymph node value for the ID-Encapsulated group is approximately 10 times the mean lymph node value obtained for the other groups. By way of comparison, the levels of nodal platinum achieved by these experiments were higher than what is disclosed in Nakamura et al. (Surgery Today, 2000), whereas a substantially lowered over-all dose of platinum was used in the current experiments.

FIGS. 5A through SD shows that kidney, liver, and lung levels of Pt were negligible for the ID-Encapsulated group. Liver samples processed from the animals receiving encapsulated drug were processed with 50% less reagent than used to process liver obtained from animals receiving free carboplatin. The plot of liver Pt does not reflect the change in sample processing volumes that could increase liver Pt values for “ID Enc” and “IV Enc”. As a control, the diluent (buffer) used to prep liposomes for injection was delivered ID and IV, one rodent per route; similar biological samples were collected and found to contain negligible levels of Pt. With further experimentation, a lymph to organ ratio (index), specifically a lymph node to spleen ratio of 30.55 was obtained (9.55 ug Pt/gram of node tissue to 0.31 ug of Pt/gram of spleen tissue), and a lymph node to kidney ratio of 55.54 (9.55 ug Pt/gram of node tissue to 0.17 ug Pt/gram for kidney tissue) was obtained. Accordingly, the particulate formulations, devices and methods of this invention can yield a lymph to organ ratio of drug equal to or greater than 10, equal to or greater than 20, equal to or greater than 30, equal to or greater than 40 and most preferably a ratio or index of 50. In addition, the amount of particulate agent remaining at the injection site was minimized. The lowest amount of agent remaining at the injection site at 24 hours was 9% of the original dose.

6.11 Systemic Delivery of Decoy Particles Prior to Intradermal Delivery of Platinum Based Drug

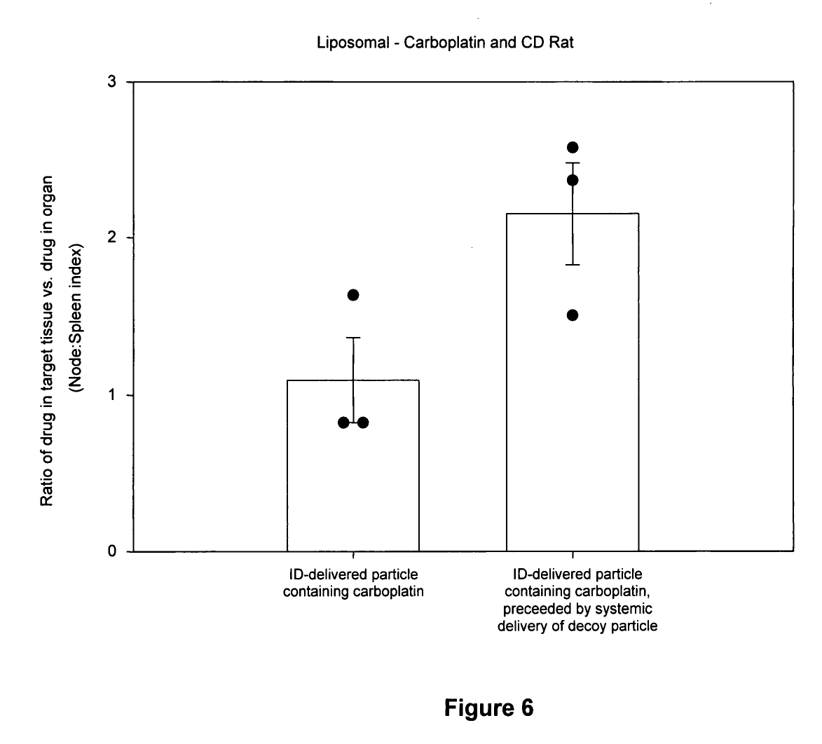

Carboplatin-liposome (F60) formed with 31.9 mg cholesterol, 95.8 mg HSPC and 31.9 mg PE-PEG2000 (1:2:1), being approximately 217 nm in diameter, was administered intradermally to six CD Rats around the rodent trunk, via a 34 g×1 mm needle. A total injection volume of 200 μl (4×50μl) was administered at an injection concentration of 12 μmole/ml. Three of the animals received an earlier IP injection of another pegylated particle, F51, formed with 50.9 mg of cholesterol, 94 mg of DSPC, 2.6 mg of PE-biotin (Avanti 87028C), 1.47 mg of DSPE-Peg 350 (Avanti 880420C) and 10.3 mg of DSPG. The concentration of the decoy particle was also 12 μmole/ml phosphorus and 1 ml was delivered (5× the particles delivered ID) via a 25 g×½ inch needle four hours in advance of the ID delivery. Twenty-four hours after the ID injection, spleen and lymph node (Ax and SI sets) tissue were collected and processed for ICP-MS as described in Example 6. 10. The tissue platinum results from animals receiving the liposomal-carboplatin via ID administration and decoy particle via IP administration were compared to values obtained from animals receiving only the liposomal-carboplatin via ID administration. The raw ICP-MS values were translated into μg of Pt/gram of node tissue (wet weight) and μg of Pt/gram of spleen tissue (wet weight). For each animal the node value was divided by the spleen value to generate a ratio or index value. The individual animal index values are shown in FIG. 6. The bars represent the mean index value for the group (n=3). Animals receiving the decoy particle produced an improved mean index, indicating a decrease in organ platinum concentration.

6.12 Detection of Encapsulated Antibody Based Drug Via Elisa

Herceptin (trastuzumab) is a recombinant DNA-derived humanized monoclonal antibody that specifically binds with high affinity to the extracellular domain of the human epidermal growth factor receptor 2 protein, HER2. Herceptin™ is a single agent treatment for patients with metastatic breast cancer. Herceptin was encapsulated via a negatively charged liposome formulation, F50, containing 57.5 mg DSPG, 50.9 mg cholesterol, 47 mg DSPC, and 2.6 mg PE-biotin. ID deliveries were performed in CD rats with a 34×1 mm microneedle. Each rodent received 105 μg of Herceptin, in both free and encapsulated form. ID injection was given along the side of the rat, close to the last rib and draining forward primarily to the axillary node. IV delivery was achieved through the tail vein. Accordingly, several nodes equally close to the IV delivery site (superficial inguinal/popliteal) were harvested as controls. The left and ride side chain axillary nodes, along with the control nodes, were harvested 24 hours after administration of free and liposomal-Herceptin, for both IV and ID routes of delivery. One hundred microliters of lysis buffer (pH 8.0), comprising 1 mM phenylmethanesulfonly fluoride, 0.25 M Tris (hydroxymethyl) aminomethane hydrochloride, 0.05 M sodium chloride, 0.5% v/v nonident P40, 0.5% w/v sodium deoxycholate, 2.5% v/v Triton-X 100, was added to the total nodal tissue collected from each rodent. The contents were homogenized in a microtube homogenizer (manually) followed by sonication three times on ice with a probe tip sonicator. The contents were microfuged to pellet insoluble material and the supernatant was removed and prepared for ELISA. Each sample lysate was diluted 1:10, and then serially 1:2 out to 1:80 in 0.5% w/v non-fat dry milk/1% v/v goat serum in TBS with Tween-20, in order to fit on a standard curve. To create a standard curve, Herceptin antibody was used at 1235 ng/well, 411.67 ng/well, 137.2 ng/well, 45.7 ng/well, 15.2 ng/well, 5.1 ng/well, 1.7 ng/well and a naive node lysate sample was prepared for a blank at 1:10 dilution. The wells of 96-well plates were first coated with 100 μl of 10 μg/ml Sigma goat anti-mouse IgG solution for 1 hour at 37° C. Non-specific sites were then blocked with 350 μl of 5% non-fat dry milk/10% goat serum solution in PBS/Tween20 for 2 hours at 37° C. After washing three times with PBS/Tween20, 100 μl of the diluted samples lysates and Herceptin antibody were added to the wells and incubated for 1 hour at 37° C. After washing three times with PBS/Tween20, 100 μl of HRP conjugate antibody, mouse anti-human IgG, (1:1000 in 0.5% non-fat dry mild/1% goat serum) was added to each well and incubated for an hour at 37° C. After washing three times, 100 μl of TMB substrate was added to each well and incubated in the dark for 30 minutes. 200 μl of 0.5 M H₂SO₄ was added to each well and the absorbance was read on a Tecan plate reader at 450 nM. The results in FIG. 7 indicate that 500-1,000 ng Herceptin per mg of axillary lymph node protein was achieved when ID delivery was combined with encapsulated technology. The highest amount of Herceptin was achieved by combining ID delivery, encapsulation and co-injection of avidin to aggregate the lipid particles in-vivo. Free Herceptin delivered IV and ID and encapsulated Herceptin delivered IV go undetected in the node.

6.13 Remote Imaging of NIR-Particles

Free cardiogreen was prepared at 80 ug/mL, and 200 uL was administered (4×50 uL) via intradermal delivery around the rodent trunk. Particulate (liposome) ICG was delivered in a similar delivery around the rodent trunk. The ID devices used were fixed with a single 34-gauge×1 mm needle. IV delivery of free and particulate ICG was administered in a 200 uL volume in a single tail vein injection. The two CD rats receiving the intradermal 50 uL×4 injection of encapsulated and free cardiogreen both manifested evidence of accumulation of the cardiogreen in the axillary and superficial inguinal node chains at 90 minutes and 4 hours post-intradermal injection. Neither of the CD rats receiving the free or encapsulated cardiogreen intradermally manifested any evidence of accumulation in the organs of the peritoneal cavity. The two CD rats receiving free and encapsulated cardiogreen intravenously manifested accumulation across the peritoneal cavity, presumably accumulation in organs such as the liver. Neither of the CD rats demonstrated any detectable evidence of accumulation in the lymph nodes at 90 minutes or 4 hours post-intravenous administration. Throughout all studies, the particles according to the invention produced acceptable draize scores at the injection sites 24 hours after the delivery (always 2 or less for edema and erythema).

All of the references cited herein are incorporated by reference in their entirety. While the invention has been described with respect to the particular embodiments, it will be apparent to those skilled in the art that various changes and modifications may be made without departing from the spirit and scope of the invention as recited by the appended claims.

The embodiments of the invention described above are intended to be merely exemplary, and those skilled in the art will recognize, or will be able to ascertain using no more than routine experimentation, numerous equivalents of specific compounds, materials, and procedures. All such equivalents are considered to be within the scope of the invention and are encompassed by the appended claims.

What is claimed is:

1. A liposome particle comprising a diagnostic or therapeutic agent, wherein said liposome particle has a zeta potential of −1 to −80 mV and a diameter of 10 to 300 nm.

2. The liposome particle of claim 1, wherein the liposome particle has a zeta-potential of about −30 to −50 mV.

3. The liposome particle of claim 1, wherein the liposome particle comprises cholesterol and at least one lipid selected from the group consisting of: DSPA, DSPC, DSPG, DSPE, HSPC, DSPE-MPEG2000, DSPE-PEG350, DPPG, DOPC, sphingomyelin, dihydrosphingomyelin, alpha tocopherol, triolein and Diol-Biotin.

4. The liposome particle of claim 3, wherein said liposome particle contains: 10% to 50% cholesterol by weight; 1 to 80% by weight of at least one lipid selected from the group consisting of DSPA, DSPC, DSPG, DSPE, HSPC, DSPE-MPEG2000, DSPE-PEG350, DPPG, DOPC, sphingomyelin, dihydrosphingomyelin, alpha tocopherol, triolein and Diol-Biotin; and less than or equal to 10% by weight of tocopherol or triolein.

5. The liposome particle of claim 1, wherein the surface of the liposome particle is coated with a binder molecule.

6. The liposome particle of claim 5, wherein the binder molecule is biotin, an antibody or an antibody fragment.

7. The liposome particle of claim 1, wherein the liposome particle contains 0.8 to 20 molar percent of said agent.

8. The liposome particle of claim 1 comprising a diagnostic agent, wherein the diagnostic agent is a dye.

9. The liposome particle of claim 8, wherein said dye is an NIR dye or SRB.

10. The liposome particle of claim 1 comprising a therapeutic agent, wherein the therapeutic agent is a chemotoxic agent or a cytokine.

11. A composition comprising a plurality of the liposome particles of claim 1, wherein the liposome particles are present in an amount of about 1 to 24 μmole/ml phosphorus.

12. The composition of claim 11, wherein the liposome particles are present in an amount of about 12 to 24 μmole/ml phosphorus.

13. A kit comprising a plurality of the liposome particles of claim 1 and a needle for intradermally injecting the liposome particles into the skin of a subject.

14. The kit of claim 13, wherein the needle is 30-34 gauge.

15. The kit of claim 13, wherein the needle is about 1 to 2 mm in length.

16. A method of delivering a diagnostic or therapeutic agent to lymphatic tissue of a subject comprising delivering a plurality of the liposome particles of claim 1 to the intradermal compartment of the subject's skin.

17. A method of delivering a diagnostic or therapeutic agent to lymphatic tissue of a subject comprising delivering the composition of claim 11 to the intradermal compartment of the subject's skin.

18. A lipid-based emulsion or liposomal composition, comprising:
(a) cholesterol and at least one lipid selected from the group consisting of: DSPA, DSPC, DSPG, DSPE, HSPC, DSPE-MPEG2000, DSPE-PEG350, DPPG, DOPC, sphingomyelin, dihydrosphingomyelin, alpha tocopherol, triolein and Diol-Biotin; and
(b) an entrapped entrapped diagnostic or therapeutic agent, wherein said composition has a zeta potential of −1 to −80 mV.

19. The composition of claim 18, wherein (a) contains: 10% to 50% cholesterol by weight; 1 to 80% by weight of at least one lipid selected from the group consisting of DSPA, DSPC, DSPG, DSPE, HSPC, DSPE-MPEG2000, DSPE-PEG350, DPPG, DOPC, sphingomyelin, dihydrosphingomyelin, alpha tocopherol, triolein and Diol-Biotin; and less than or equal to 10% by weight of tocopherol or triolein.

20. A method of delivering a diagnostic or therapeutic agent to lymphatic tissue of a subject, comprising delivering particles containing said agent to the intradermal compartment of the subject's skin, introducing to the subject a condition that causes said particles to transform after said particles are delivered to the intradermal compartment, wherein transformation of said particles facilitates retention of said particles by the lymphatic tissue.

21. The method of claim 20, wherein the particles are sacs, microcapsules, liposomes, dendrimers, nanoshells, quantum dots, or fullerene.

22. The method of claim 20, wherein the particles are liposomes that comprise cholesterol and at least one lipid selected from the group consisting of: DSPA, DSPC, DSPG, DSPE, HSPC, DSPE-MPEG2000, DSPE-PEG350, DPPG, DOPC, sphingomyelin, dihydrosphingomyelin, alpha tocopherol, triolein and Diol-Biotin.

23. The method of claim 20, wherein the particles are liposomes and wherein the surfaces of the liposome particles are coated with a binder molecule.

24. The method of claim 23, wherein introducing a condition that causes said particles to transform comprises delivering a corresponding binder to the intradermal compartment of the subject's skin before delivering the liposomes to the intradermal compartment.

25. The method of claim 24, wherein said binder molecule is biotin and said corresponding binder is avidin, or wherein said binder molecule is an antibody or antibody fragment and said corresponding binder is an antigen or antigen fragment.

26. The method of claim 20, wherein introducing a condition that causes said particles to transform comprises introducing a condition that causes said particles to aggregate.

27. The method of claim 20, wherein the condition that causes particles to transform is heat, sonowaves, magnetic force, or the introduction of a condensing agent.

28. The method of claim 20, wherein the particles are delivered using a needle of about 1 to 2 mm in length.

29. The method of claim 20, wherein the particles are liposomes having a diameter of 10 to 300 nm.

30. The method of claim 20, wherein the particles are liposomes having a zeta-potential of about −1 to −80 mV.

31. The method of claim 20, wherein delivery of the agent to the lymphatic tissue of the subject results in a lymph-to-organ ratio of agent concentration equal to or greater than 10.
